SEQUENCE LISTING (2 sequences)

SEQ ID NO: 1 - protein, 124 aa, Artificial Sequence
  VHH 7D12
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 2 - protein, 127 aa, Artificial Sequence
  VHH 9G8
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

(19) United States
(12) Patent Application Publication
(10) Pub. No.: US 2023/0248845 A1
(43) Pub. Date: Aug. 10, 2023

(54) COMBINATION OF AN ANTIBODY-DRUG CONJUGATE AND AN ANTIBODY-SAPONIN CONJUGATE

(71) Applicant: SAPREME TECHNOLOGIES B.V., Bilthoven (NL)

(72) Inventors: Ruben POSTEL, Bilthoven (NL); Guy HERMANS, Bilthoven (NL)

(21) Appl. No.: 18/012,760

(22) PCT Filed: Jun. 22, 2021
(86) PCT No.: PCT/NL2021/050391

§ 371 Date: Dec. 23, 2022

(30) Foreign Application Priority Data
Jun. 24, 2020 (NL) 2025905

(51) Int. Cl. A61K 47/68; A61K 47/54; A61P 35/00

(52) U.S. Cl. CPC A61K 47/6851; A61K 47/549; A61K 47/542; A61K 47/6815; A61K 47/6827; A61K 47/6817; A61K 47/6829; A61K 47/6809; A61K 47/6889; A61P 35/00

(57) ABSTRACT

The invention relates to a therapeutic combination comprising: (a) a first pharmaceutical composition comprising a conjugate comprising a first binding molecule for binding to a first binding site of a cell-surface molecule and the conjugate comprising a saponin bound to said first binding molecule, wherein the saponin is a triterpene glycoside; and (b) a second pharmaceutical composition comprising a conjugate comprising a second binding molecule different from the first binding molecule, the second binding molecule comprising a second binding region different from the first binding region, for binding to a second binding site of said cell-surface molecule different from the first binding site of said cell-surface molecule, and the conjugate comprising an effector molecule covalently bound to said second binding molecule. The invention also relates to a pharmaceutical composition comprising said two conjugates. In addition, the invention relates to the pharmaceutical combination or the pharmaceutical composition of the invention for use as a medicament. Furthermore, the invention relates to the pharmaceutical combination or the pharmaceutical composition of the invention, for use in the treatment or prevention of a cancer, an autoimmune disease, a disease relating to (over)expression of a protein, a disease relating to an aberrant cell such as a tumor cell or a diseased liver cell, a disease relating to a mutant gene, a disease relating to a gene defect, a disease relating to a mutant protein, a disease relating to absence of a (functional) protein, a disease relating to a (functional) protein deficiency.

### TECHNOLOGICAL FIELD

The invention relates to a therapeutic combination comprising: (a) a first pharmaceutical composition comprising a conjugate comprising a first binding molecule comprising a first binding region for binding to a first binding site of a cell-surface molecule and the conjugate comprising at least one saponin covalently bound to said first binding molecule; and (b) a second pharmaceutical composition comprising a conjugate comprising a second binding molecule different from the first binding molecule, the second binding molecule comprising a second binding region different from the first binding region, the second binding region for binding to a second binding site of said cell-surface molecule different from the first binding site of said cell-surface molecule, and the conjugate comprising an effector molecule covalently bound to said second binding molecule. The invention also relates to a pharmaceutical composition comprising said two conjugates. In addition, the invention relates to the pharmaceutical combination or the pharmaceutical composition of the invention for use as a medicament. Furthermore, the invention relates to the pharmaceutical combination or the pharmaceutical composition of the invention, for use in the treatment or prevention of a cancer, an autoimmune disease, a disease relating to (over)expression of a protein, a disease relating to an aberrant cell such as a tumor cell or a diseased liver cell, a disease relating to a mutant gene, a disease relating to a gene defect, a disease relating to a mutant protein, a disease relating to absence of a (functional) protein, a disease relating to a (functional) protein deficiency.

### BACKGROUND

Molecules with a therapeutic biological activity are in many occasions in theory suitable for application as an effective therapeutic drug for the treatment of a disease such as a cancer in human patients in need thereof. A typical example are small-molecule biologically active moieties. However, many if not all potential drug-like molecules and therapeutics currently used in the clinic suffer from at least one of a plethora of shortcomings and drawbacks. When administered to a human body, therapeutically active molecules may exert off-target effects, in addition to the biologically activity directed to an aspect underlying a to-be-treated disease or health problem. Such off-target effects are undesired and bear a risk for induction of health- or even life-threatening side effects of the administered molecule. It is the occurrence of such adverse events that cause many drug-like compounds and therapeutic moieties to fail phase III clinical trials or even phase IV clinical trials (post-market entry follow-up). Therefore, there is a strong desire to provide drug molecules such as small-molecule therapeutics, wherein the therapeutic effect of the drug molecule should, e.g., (1) be highly specific for a biological factor or biological process driving the disease, (2) be sufficiently safe, (3) be sufficiently efficacious, (4) be sufficiently directed to the diseased cell with little to no off-target activity on non-diseased cells, (5) have a sufficiently timely mode of action (e.g. the administered drug molecule should reach the targeted site in the human patient within a certain time frame and should remain at the targeted site for a certain time frame), and/or (6) have sufficiently long lasting therapeutic activity in the patient's body, amongst others. Unfortunately, to date, ‘ideal’ therapeutics with many or even all of the beneficial characteristics here above outlined, are not available to the patients, despite already long-lasting and intensive research and despite the impressive progress made in several areas of the individually addressed encountered difficulties and drawbacks.

Chemotherapy is one of the most important therapeutic options for cancer treatment. However, it is often associated with a low therapeutic window because it has no specificity towards cancer cells over dividing cells in healthy tissue. The invention of monoclonal antibodies offered the possibility of exploiting their specific binding properties as a mechanism for the targeted delivery of cytotoxic agents to cancer cells, while sparing normal cells. This can be achieved by chemical conjugation of cytotoxic effectors (also known as payloads or warheads) to antibodies, to create antibody—drug conjugates (ADCs). Typically, very potent payloads such as emtansine (DM1) are used which have a limited therapeutic index (a ratio that compares toxic dose to efficacious dose) in their unconjugated forms. The conjugation of DM1 to trastuzumab (ado-trastuzumab emtansine), also known as Kadcycla, enhances the tolerable dose of DM1 at least two-fold in monkeys. In the past few decades tremendous efforts and investments have been made to develop therapeutic ADCs. However, it remains challenging to bring ADCs into the clinic, despite promising preclinical data. The first ADC approved for clinical use was gemtuzumab ozogamicin (Mylotarg, CD33 targeted, Pfizer/Wyeth) for relapsed acute myelogenous leukemia (AML) in 2000. Mylotarg was however, withdrawn from the market at the request of the Federal Drug Administration (FDA) due to a number of concerns including its safety profile. Patients treated with Mylotarg were more often found to die than patients treated with conventional chemotherapy. Mylotarg was admitted to the market again in 2017 with a lower recommended dose, a different schedule in combination with chemotherapy or on its own, and a new patient population. To date, only five ADCs have been approved for clinical use, and meanwhile clinical development of approximately fifty-five ADCs has been halted. However, interest remains high and approximately eighty ADCs are still in clinical development in nearly six-hundred clinical trials at present.

Despite the potential to use toxic payloads that are normally not tolerated by patients, a low therapeutic index (a ratio that compares toxic dose to efficacious dose) is a major problem accounting for the discontinuance of many ADCs in clinical development, which can be caused by several mechanisms such as off-target toxicity on normal cells, development of resistance against the cytotoxic agents and premature release of drugs in the circulation. A systematic review by the FDA of ADCs found that the toxicity profiles of most ADCs could be categorized according to the payload used, but not the antibody used, suggesting that toxicity is mostly determined by premature release of the payload. Of the approximately fifty-five ADCs that were discontinued, it is estimated that at least twenty-three were due to a poor therapeutic index. For example, development of a trastuzumab tesirine conjugate (ADCT-502, HER-2 targeted, ADC therapeutics) was recently discontinued due to a narrow therapeutic index, possibly due to an on-target, off-tissue effect in pulmonary tissue which expresses considerable levels of HER-2. In addition, several ADCs in phase 3 trials have been discontinued due to missing primary endpoint. For example, phase 3 trials of a depatuxizumab mafodotin conjugate (ABT-414, EGFR targeted, AbbVie) tested in patients with newly diagnosed glioblastoma, and a mirvetuximab soravtansine conjugate (IMGN853, folate receptor alpha (FRα) targeted, ImmunoGen) tested in patients with platinum-resistant ovarian cancer, were recently stopped, showing no survival benefit. It is important to note that the clinically used dose of some ADCs may not be sufficient for its full anticancer activity. For example, ado-trastuzumab emtansine has an MTD of 3.6 mg/kg in humans. In preclinical models of breast cancer, ado-trastuzumab emtansine induced tumor regression at dose levels at or above 3 mg/kg, but more potent efficacy was observed at 15 mg/kg. This suggests that at the clinically administered dose, ado-trastuzumab emtansine may not exert its maximal potential anti-tumor effect.

ADCs are mainly composed of an antibody, a cytotoxic moiety such as a payload, and a linker. Several novel strategies have been proposed and carried out in the design and development of new ADCs to overcome the existing problems, targeting each of the components of ADCs. For example, by identification and validation of adequate antigenic targets for the antibody component, by selecting antigens which have high expression levels in tumor and little or no expression in normal tissues, antigens which are present on the cell surface to be accessible to the circulating ADCs, and antigens which allows internalizing of ADCs into the cell after binding; and alternative mechanisms of activity; design and optimize linkers which enhance the solubility and the drug-to-antibody ratio (DAR) of ADCs and overcome resistance induced by proteins that can transport the chemotherapeutic agent out of the cells; enhance the DAR ratio by inclusion of more payloads, select and optimize antibodies to improve antibody homogeneity and developability. In addition to the technological development of ADCs, new clinical and translational strategies are also being deployed to maximize the therapeutic index, such as, change dosing schedules through fractionated dosing; perform biodistribution studies; include biomarkers to optimize patient selection, to capture response signals early and monitor the duration and depth of response, and to inform combination studies.

An example of ADCs with clinical potential are those ADCs such as brentuximab vedotin, inotuzumab ozogamicin, moxetumomab pasudotox, and polatuzumab vedotin, which are evaluated as a treatment option for lymphoid malignancies and multiple myeloma. Polatuzurnab vedotin, binding to CD79b on (malignant) B-cells, and pinatuzumab vedotin, binding to CD22, are tested in clinical trials wherein the ADCs each were combined with co-administered rituximab, a monoclonal antibody binding to CD20 and not provided with a payload [B. Yu and D. Liu, Antibody-drug conjugates in clinical trials for lymphoid malignancies and multiple myeloma; Journal of Hematology & Oncology (2019) 12:94]. Combinations of monoclonal antibodies such as these examples are yet a further approach and attempt to arrive at the ‘magic bullet’ which combines many or even all of the aforementioned desired characteristics of ADCs.

Meanwhile in the past few decades, nucleic acid-based therapeutics are under development. Therapeutic nucleic acids can be based on deoxyribonucleic acid (DNA) or ribonucleic acid (RNA), Anti-sense oligonucleotides (ASOs, AONs), and short interfering RNAs (siRNAs), MicroRNAs, and DNA and RNA aptamers, for approaches such as gene therapy, RNA interference (RNAi). Many of them share the same fundamental basis of action by inhibition of either DNA or RNA expression, thereby preventing expression of disease-related abnormal proteins. The largest number of clinical trials is being carried out in the field of gene therapy, with almost 2600 ongoing or completed clinical trials worldwide but with only about 4% entering phase 3. This is followed by clinical trials with ASOs. Similarly to ADCs, despite the large number of techniques being explored, therapeutic nucleic acids share two major issues during clinical development: delivery into cells and off-target effects. For instance, ASOs such as peptide nucleic acid (PNA), phosphoramidate morpholino oligomer (PMO), locked nucleic acid (LNA) and bridged nucleic acid (BNA), are being investigated as an attractive strategy to inhibit specifically target genes and especially those genes that are difficult to target with small molecules inhibitors or neutralizing antibodies. Currently, the efficacy of different ASOs is being studied in many neurodegenerative diseases such as Huntington's disease, Parkinson's disease, Alzheimer's disease, and amyotrophic lateral sclerosis and also in several cancer stages. The application of ASOs as potential therapeutic agents requires safe and effective methods for their delivery to the cytoplasm and/or nucleus of the target cells and tissues. Although the clinical relevance of ASOs has been demonstrated, inefficient cellular uptake, both in vitro and in vivo, limit the efficacy of ASOs and has been a barrier to therapeutic development. Cellular uptake can be <2% of the dose resulting in too low ASO concentration at the active site for an effective and sustained outcome. This consequently requires an increase of the administered dose which induces off-target effects. Most common side-effects are activation of the complement cascade, the inhibition of the clotting cascade and toll-like receptor mediated stimulation of the immune system.

Chemotherapeutics are most commonly small molecules, however, their efficacy is hampered by the severe off-target side toxicity, as well as their poor solubility, rapid clearance and limited tumor exposure. Scaffold-small-molecule drug conjugates such as polymer-drug conjugates (PDCs) are macromolecular constructs with pharmacologically activity, which comprises one or more molecules of a small-molecule drug bound to a carrier scaffold (e.g. polyethylene glycol (PEG)).

Such conjugate principle has attracted much attention and has been under investigation for several decades. The majority of conjugates of small-molecule drugs under pre-clinical or clinical development are for oncological indications. However, up-to-date only one drug not related to cancer has been approved (Movantik, a PEG oligomer conjugate of opioid antagonist naloxone, AstraZeneca) for opioid-induced constipation in patients with chronic pain in 2014, which is a non-oncology indication. Translating application of drug-scaffold conjugates into treatment of human subjects provides little clinical success so far. For example, PK1 (N-(2-hydroxypropyl)methacrylamide (HPMA) copolymer doxorubicin; development by Pharmacia, Pfizer) showed great anti-cancer activity in both solid tumors and leukemia in murine models, and was under clinical investigation for oncological indications. Despite that it demonstrated significant reduction of nonspecific toxicity and improved pharmacokinetics in man, improvements in anticancer efficacy turned out to be marginal in patients, and as a consequence further development of PK1 was discontinued.

The failure of scaffold-small-molecule drug conjugates is at least partially attributed to its poor accumulation at the tumor site. For example, while in murine models PK1 showed 45-250 times higher accumulation in the tumor than in healthy tissues (liver, kidney, lung, spleen, and heart), accumulation in tumor was only observed in a small subset of patients in the clinical trial.

A potential solution to the aforementioned problems is application of nanoparticle systems for drug delivery such as liposomes. Liposomes are sphere-shaped vesicles consisting of one or more phospholipid bilayers, which are spontaneously formed when phospholipids are dispersed in water. The amphiphilicity characteristics of the phospholipids provide it with the properties of self-assembly, emulsifying and wetting characteristics, and these properties can be employed in the design of new drugs and new drug delivery systems. Drug encapsulated in a liposomal delivery system may convey several advantages over a direct administration of the drug, such as an improvement and control over pharmacokinetics and pharmacodynamics, tissue targeting property, decreased toxicity and enhanced drug activity. An example of such success is liposome-encapsulated form of a small molecule chemotherapy agent doxorubicin (Doxil: a pegylated liposome-encapsulated form of doxorubicin; Myocet: a non-pegylated liposomal doxorubicin), which have been approved for clinical use.

Therefore, a solution still needs to be found that allows for drug therapies such as anti-tumor therapies, applicable for non-systemic use when desired, wherein the drug has for example an acceptable safety profile, little off-target activity, sufficient efficacy, sufficiently low clearance rate from the patient's body, a sufficiently wide therapeutic window, etc.

### SUMMARY

A first aspect of the invention relates to a therapeutic combination comprising:
(a) a first pharmaceutical composition comprising a conjugate comprising a first binding molecule comprising a first binding region for binding to a first binding site of a cell-surface molecule and the conjugate comprising at least one saponin covalently bound to said first binding molecule, wherein the saponin is a monodesmosidic triterpene glycoside or a bidesmosidic triterpene glycoside; and
(b) a second pharmaceutical composition comprising a conjugate comprising a second binding molecule different from the first binding molecule, the second binding molecule comprising a second binding region different from the first binding region, the second binding region for binding to a second binding site of said cell-surface molecule different from the first binding site of said cell-surface molecule, and the conjugate comprising an effector molecule covalently bound to said second binding molecule,
the first pharmaceutical composition and the second pharmaceutical composition optionally further comprising a pharmaceutically acceptable excipient and optionally further comprising a pharmaceutically acceptable diluent.

A second aspect of the invention relates to a pharmaceutical composition comprising:
a conjugate comprising a first binding molecule comprising a first binding region for binding to a first binding site of a cell-surface molecule and the conjugate comprising at least one saponin covalently bound to said first binding molecule, wherein the saponin is a triterpenoid saponin of the monodesmosidic type or the bidesmosidic type; and
a conjugate comprising a second binding molecule different from said first binding molecule, the second binding molecule comprising a second binding region different from said first binding region, the second binding region for binding to a second binding site of said cell-surface molecule different from said first binding site of said cell-surface molecule, and the conjugate comprising an effector molecule covalently bound to said second binding molecule,
   and optionally further comprising a pharmaceutically acceptable excipient and optionally further comprising a pharmaceutically acceptable diluent.

A third aspect of the invention relates to a pharmaceutical combination of the invention, or a pharmaceutical composition of the invention, for use as a medicament.

A fourth aspect of the invention relates to a pharmaceutical combination of the invention, or a pharmaceutical composition of the invention, for use in the treatment or prevention of a cancer, an autoimmune disease, a disease relating to (over)expression of a protein, a disease relating to an aberrant cell such as a tumor cell or a diseased liver cell, a disease relating to a mutant gene, a disease relating to a gene defect, a disease relating to a mutant protein, a disease relating to absence of a (functional) protein, a disease relating to a (functional) protein deficiency.

A fifth aspect of the invention relates to a kit of parts, comprising the pharmaceutical combination of the invention or comprising the pharmaceutical composition of the invention, and optionally instructions for use of said pharmaceutical combination or said pharmaceutical composition.

### Definitions

The term “binding region” has its regular scientific meaning and here refers to a part of a molecule or (a) chemical group(s) of a molecule or a(n) (linear or non-linear) amino-acid sequence of a protein or peptide and the like, that has the capacity to bind to a binding partner molecule. A typical binding region are the CDR loops of an immunoglobulin. A typical binding region of a protein is or are loop(s) of amino-acid residues comprised by said protein and capable of specifically binding to the binding site on a binding partner molecule such as a protein, cell-surface receptor, etc.

The term “binding site” has its regular scientific meaning and here refers to a region on a macromolecule such as a protein, for example a cell-surface molecule such as a cell-surface receptor, that binds to another molecule such as a protein, for example a ligand, with specificity.

The term “cell-surface molecule” has its regular scientific meaning and here refers to a molecule that is present and exposed at the outside surface of a cell such as a blood cell or an organ cell, such as a mammalian cell, such as a human cell. Typically, a cell-surface molecule is a protein such as a receptor, or a lipid molecule or a polysaccharide.

The term “saponin” has its regular scientific meaning and here refers to a group of amphipatic glycosides which comprise one or more hydrophilic glycone moieties combined with a lipophilic aglycone core which is a sapogenin. The saponin may be naturally occurring or synthetic (i.e. non-naturally occurring). The term “saponin” includes naturally-occurring saponins, derivatives of naturally-occurring saponins as well as saponins synthesized de novo through chemical and/or biotechnological synthesis routes. Saponin has a triterpene backbone, which is a pentacyclic C30 terpene skeleton, also referred to as sapogenin or aglycone. Within the context of the invention saponin is not considered an effector molecule nor an effector moiety in the conjugates according to the invention. Thus, in conjugates comprising a saponin and an effector moiety, the effector moiety is a different molecule than the conjugated saponin.

The term “aglycone core structure”, also referred to as “sapogenin” or as “aglycone core” or as “aglycone”, has its regular scientific meaning and here refers to the aglycone core of a saponin without the one or two carbohydrate antenna or saccharide chains (glycans) bound thereto. For example, quillaic acid is the aglycone core structure for SO1861, QS-7, QS21.

The term “saccharide chain” has its regular scientific meaning and here refers to any of a glycan, a carbohydrate antenna, a single saccharide moiety (monosaccharide) or a chain comprising multiple saccharide moieties (oligosaccharide, polysaccharide). The saccharide chain can consist of only saccharide moieties or may also comprise further moieties such as any one of 4E-Methoxycinnamic acid, 4Z-Methoxycinnamic acid, and 5-O-[5-O-Ara/Api-3,5-dihydroxy-6-methyl-octanoyl]-3,5-dihydroxy-6-methyl-octanoic acid), such as for example present in QS-21.

The term “mono-desmosidic saponin” has its regular scientific meaning and here refers to a triterpenoid saponin containing a single saccharide chain bound to the aglycone core, wherein the saccharide chain consists of one or more saccharide moieties.

The term “bi-desmosidic saponin” has its regular scientific meaning and here refers to a triterpenoid saponin containing two saccharide chains bound to the aglycone core, wherein each of the two saccharide chains consists of one or more saccharide moieties.

The term “triterpenoid saponin” has its regular scientific meaning and here refers to a saponin having a triterpenoid-type of aglycone core structure. The triterpenoid saponin differs from a saponin based on a steroid glycoside such as sapogenol in that such saponin comprising steroid glycoside has a steroid core structure, and the triterpenoid saponin differs from a saponin based on an alkaloid glycoside such as tomatidine in that such saponin comprising alkaloid glycoside has a alkaloid core structure.

The term “antibody-drug conjugate” or “ADC” has its regular scientific meaning and here refers to any conjugate of an antibody such as an IgG, a Fab, an scFv, an immunoglobulin, an immunoglobulin fragment, one or multiple V_{H} domains, etc., and any molecule that can exert a therapeutic effect when contacted with cells of a subject such as a human patient, such as an active pharmaceutical ingredient, a toxin, an oligonucleotide, an enzyme, a small molecule drug compound, etc.

The term “antibody-oligonucleotide conjugate” or “AOC” has its regular scientific meaning and here refers to any conjugate of an antibody such as an IgG, a Fab, an scFv, an immunoglobulin, an immunoglobulin fragment, one or multiple V_{H} domains, etc., and any oligonucleotide molecule that can exert a therapeutic effect when contacted with cells of a subject such as a human patient, such as an oligonucleotide selected from a natural or synthetic string of nucleic acids encompassing DNA, modified DNA, RNA, modified RNA, synthetic nucleic acids, presented as a single-stranded molecule or a double-stranded molecule, such as a BNA, an allele-specific oligonucleotide (ASO), a short or small interfering RNA (siRNA; silencing RNA), an anti-sense DNA, anti-sense RNA, etc.

The term “conjugate” has its regular scientific meaning and here refers to at least a first molecule that is covalently bound through chemical bonds to at least a second molecule, therewith forming an covalently coupled assembly comprising or consisting of the first molecule and the second molecule. Typical conjugates are an ADC, an AOC, and SO1861-EMCH (EMCH linked to the aldehyde group of the aglycone core structure of the saponin).

The term “single-domain antibody”, or “sdAb”, in short, has its regular scientific meaning and here refers to an antibody fragment consisting of a single monomeric variable antibody domain. In the conjugates of the invention, more than one sdAb can be present, which sdAb's can be the same (multivalent and mono-specific) or can be different (multivalent and/or for example multi-paratope, bi-paratope, multi-specific, bi-specific). In addition, for example the more than two sdAb's are for example a combination of mono-specific and multivalent sdAb's and at least one further sdAb that binds to a different epitope (e.g. multispecific or biparatope).

The term “effector molecule”, or “effector moiety” when referring to the effector molecule as part of e.g. a covalent conjugate, has its regular scientific meaning and here refers to a molecule that can selectively bind to for example any one or more of the target molecules: a protein, a peptide, a carbohydrate, a saccharide such as a glycan, a (phospho)lipid, a nucleic acid such as DNA, RNA, an enzyme, and regulates the biological activity of such one or more target molecule(s). In the conjugate of the invention the effector moiety for example exerts its effect in the cytosol, in the cell nucleus, is delivered intracellularly in the endosome and/or lysosome, and/or is active after exiting or escaping the endosomal-lysosomal pathway. The effector molecule is for example a molecule selected from any one or more of a small molecule such as a drug molecule, a toxin such as a protein toxin, an oligonucleotide such as a BNA, a xeno nucleic acid or an siRNA, an enzyme, a peptide, a protein, or an active fragment or an active domain thereof, or any combination thereof. Thus, for example, an effector molecule or an effector moiety is a molecule or moiety selected from any one or more of a small molecule such as a drug molecule, a toxin such as a protein toxin, an oligonucleotide such as a BNA, a xeno nucleic acid or an siRNA, an enzyme, a peptide, a protein, or any combination thereof, that can selectively bind to any one or more of the target molecules: a protein, a peptide, a carbohydrate, a saccharide such as a glycan, a (phospho)lipid, a nucleic acid such as DNA, RNA, an enzyme, and that upon binding to the target molecule regulates the biological activity of such one or more target molecule(s). For example, an effector moiety is a toxin or an active toxic fragment thereof or an active toxic derivative or an active toxic domain thereof. Typically, an effector molecule can exert a biological effect inside a cell such as a mammalian cell such as a human cell, such as in the cytosol of said cell. An effector molecule or moiety of the invention is thus any substance that affects the metabolism of a cell by interaction with an intracellular effector molecule target, wherein this effector molecule target is any molecule or structure inside cells excluding the lumen of compartments and vesicles of the endocytic and recycling pathway but including the membranes of these compartments and vesicles. Said structures inside cells thus include the nucleus, mitochondria, chloroplasts, endoplasmic reticulum, Golgi apparatus, other transport vesicles, the inner part of the plasma membrane and the cytosol. Typical effector molecules are thus drug molecules, an enzyme, plasmid DNA, toxins such as toxins comprised by antibody-drug conjugates (ADCs), oligonucleotides such as siRNA, BNA, nucleic acids comprised by an antibody-oligonucleotide conjugate (AOC). For example, an effector molecule is a molecule which can act as a ligand that can increase or decrease (intracellular) enzyme activity, gene expression, or cell signalling. In the context of the invention, an effector molecule or effector moiety when the effector molecule is part of a conjugate, is not a saponin, and is not a cell-surface molecule binding molecule such as an antibody such as an sdAb. Typically, an effector moiety comprised by the conjugate exerts its therapeutic (for example toxic, enzymatic, inhibitory, gene silencing, etc.) effect in the cytosol and/or in the cell nucleus. Typically, the effector moiety is delivered intracellularly in the endosome and/or in the lysosome, and typically the effector moiety is active after exiting or escaping the endosomal-lysosomal pathway.

The term “tumor cell-specific surface molecule” and the term “tumor cell-specific receptor” have their regular scientific meaning and here refer to a molecule or a receptor that is expressed and exposed at the surface of a tumor cell and not at the surface of a healthy, non-cancerous cell, or is expressed at the surface of a healthy, non-cancerous cell to a lower extent than the level of expression (number of molecules/receptors) at the surface of the tumor cell.

The term “payload” has its regular scientific meaning and here refers to a biologically active molecule such as for example a cytotoxic (anti-cancer) drug molecule.

The term “oligonucleotide” has its regular scientific meaning and here refers to a string of two or more nucleotides, i.e. an oligonucleotides is a short oligomer composed of ribonucleotides or deoxyribonucleotides. Examples are RNA and DNA, and any modified RNA or DNA, such as a string of nucleic acids comprising a nucleotide analogue such as a bridged nucleic acid (BNA), also known as locked nucleic acid (LNA) or 2′-O,4′-C-aminoethylene or 2′-O,4′-C-aminomethylene bridged nucleic acid (BNA^{NC}), wherein the nucleotide is a ribonucleotide or a deoxyribonucleotide.

The term “bridged nucleic acid”, or “BNA” in short, or “locked nucleic acid” or “LNA” in short or 2′-O,4′-C-aminoethylene or 2′-O,4′-C-aminomethylene bridged nucleic acid (BNA^{NC}), has its regular scientific meaning and here refers to a modified RNA nucleotide. A BNA is also referred to as ‘constrained RNA molecule’ or ‘inaccessible RNA molecule’. A BNA monomer can contain a five-membered, six-membered or even a seven-membered bridged structure with a “fixed” C₃′-endo sugar puckering. The bridge is synthetically incorporated at the 2′, 4′-position of the ribose to afford a 2′, 4′-BNA monomer. A BNA monomer can be incorporated into an oligonucleotide polymeric structure using standard phosphoramidite chemistry known in the art. A BNA is a structurally rigid oligonucleotide with increased binding affinity and stability.

The term “proteinaceous” has its regular scientific meaning and here refers to a molecule that is protein-like, meaning that the molecule possesses, to some degree, the physicochemical properties characteristic of a protein, is of protein, relating to protein, containing protein, pertaining to protein, consisting of protein, resembling protein, or being a protein. The term “proteinaceous” as used in for example ‘proteinaceous molecule’ refers to the presence of at least a part of the molecule that resembles or is a protein, wherein ‘protein’ is to be understood to include a chain of amino-acid residues at least two residues long, thus including a peptide, a polypeptide and a protein and an assembly of proteins or protein domains. In the proteinaceous molecule, the at least two amino-acid residues are for example linked via (an) amide bond(s), such as (a) peptide bond(s). In the proteinaceous molecule, the amino-acid residues are natural amino-acid residues and/or artificial amino-acid residues such as modified natural amino-acid residues. In a preferred embodiment, a proteinaceous molecule is a molecule comprising at least two amino-acid residues, preferably between two and about 2.000 amino-acid residues. In one embodiment, a proteinaceous molecule is a molecule comprising from 2 to 20 (typical for a peptide) amino acids. In one embodiment, a proteinaceous molecule is a molecule comprising from 21 to 1.000 (typical for a polypeptide, a protein, a protein domain, such as an antibody, a Fab, an scFv, a ligand for a receptor such as EGF) amino acids. Preferably, the amino-acid residues are (typically) linked via (a) peptide bond(s). According to the invention, said amino-acid residues are or comprise (modified) (non-)natural amino acid residues.

The term “binding molecule” has its regular scientific meaning and here refers to a molecule capable of specifically binding to another molecule such as a cell-surface molecule, e.g. a cell-surface receptor. Typical binding molecules are peptides, proteins, non-protein molecules, cell-surface receptor ligands, protein ligands, that can bind to e.g. a protein, a lipid, a (poly)saccharide, such as a cell-surface receptor or a cell-surface molecule. “Specifically binding” here refers to specific and selective binding with higher affinity than non-specific background binding.

The term “Api/Xyl-” or “Api- or Xyl-” in the context of the name of a saccharide chain has its regular scientific meaning and here refers to the saccharide chain either comprising an apiose (Api) moiety, or comprising a xylose (Xyl) moiety.

The term “moiety” has its regular scientific meaning and here refers to an molecule that is bound, linked, conjugated to a further molecule, linker, assembly of molecules, etc., and therewith forming part of a larger molecule, conjugate, assembly of molecules. Typically, an moiety is an molecule that is covalently bound to another molecules, involving one or more chemical groups initially present on the effector molecule. For example, saporin is a typical effector molecule. As part of an antibody-drug conjugate, the saporin is a typical effector moiety in the ADC. As part of an antibody-oligonucleotide conjugate, a BNA or an siRNA is a typical effector moiety in the AOC.

The terms first, second, third and the like in the description and in the claims, are used for distinguishing between for example similar elements, compositions, constituents in a composition, or separate method steps, and not necessarily for describing a sequential or chronological order. The terms are interchangeable under appropriate circumstances and the embodiments of the invention can operate in other sequences than described or illustrated herein, unless specified otherwise.

The embodiments of the invention described herein can operate in combination and cooperation, unless specified otherwise.

Furthermore, the various embodiments, although referred to as “preferred” or “e.g.” or “for example” or “in particular” and the like are to be construed as exemplary manners in which the invention may be implemented rather than as limiting the scope of the invention.

The term “comprising”, used in the claims, should not be interpreted as being restricted to for example the elements or the method steps or the constituents of a compositions listed thereafter; it does not exclude other elements or method steps or constituents in a certain composition. It needs to be interpreted as specifying the presence of the stated features, integers, (method) steps or components as referred to, but does not preclude the presence or addition of one or more other features, integers, steps or components, or groups thereof. Thus, the scope of the expression “a method comprising steps A and B” should not be limited to a method consisting only of steps A and B, rather with respect to the present invention, the only enumerated steps of the method are A and B, and further the claim should be interpreted as including equivalents of those method steps. Thus, the scope of the expression “a composition comprising components A and B” should not be limited to a composition consisting only of components A and B, rather with respect to the present invention, the only enumerated components of the composition are A and B, and further the claim should be interpreted as including equivalents of those components.

In addition, reference to an element or a component by the indefinite article “a” or “an” does not exclude the possibility that more than one of the element or component are present, unless the context clearly requires that there is one and only one of the elements or components. The indefinite article “a” or “an” thus usually means “at least one”.

### BRIEF DESCRIPTION OF THE DRAWINGS

FIG. 1 (FIG. 1) is a cartoon displaying the non-competing 1 target 2-components system (1T2C, non-competing) according to the invention, which is the combination treatment of mAb1-SO1861 and mAb2-protein toxin, where mAb1 and mAb2 both target and bind the same receptor, but recognize different epitopes on the receptor, thereby excluding mAb receptor binding competition.

FIG. 2 (FIG. 2) shows the results of the determination of cell killing on HER2 expressing cells (SK-BR-3, HER2⁺⁺) (A) and non-expressing cells (MDA-MB-468, HER2⁻) (B) under influence of free pertuzumab and free trastuzumab, or antibody conjugated to either SO1861, or saporin, and combinations thereof as indicated in the legend (Legend for FIGS. 2A and 2B is the same and is displayed next to FIG. 2B).

FIG. 3 (FIG. 3) shows the results of the determination of targeted protein toxin mediated cell killing on HER2 expressing cells (SK-BR-3, HER2⁺⁺) (A) and HER2 non-expressing cells (MDA-MB-468, HER2⁻) (B) when trastuzumab-saporin was titrated on a fixed concentration of 2.5 nM and 75 nM pertuzumab-(Cys-L-SO1861)⁴ (Legend for FIGS. 3A and 3B is the same and is displayed next to FIG. 3B).

FIG. 4 (FIG. 4) shows the results of the determination of targeted protein toxin mediated cell killing on HER2 expressing cells (SK-BR-3, HER2⁺⁺) (A) and non-expressing cells (MDA-MB-468, HER2⁻) (B) when pertuzumab-(Cys-L-SO1861)⁴ or trastuzumab-(Cys-L-SO1861)⁴ was titrated on a fixed concentration of 50 pM pertuzumab-dianthin (pertuzumab conjugated to the protein toxin, dianthin, with a DAR4) (Legend for FIGS. 4A and 4B is the same and is displayed next to FIG. 4B).

FIG. 5 (FIG. 5) shows the results of the determination of targeted protein toxin mediated cell killing on HER2 expressing cells (SK-BR-3, HER2⁺⁺) and non-expressing cells (MDA-MB-468, HER2⁻) when pertuzumab-dianthin was titrated on a fixed concentration of 2.5 nM and 25 nM pertuzumab-(Cys-L-SO1861)⁴ or trastuzumab-(Cys-L-SO1861)⁴ (Legend for FIGS. 5A and 5B is the same and is displayed next to FIG. 5B).

FIG. 6 (FIG. 6) shows the results of the determination of targeted protein toxin mediated cell killing on EGFR expressing cells (A431, EGFR⁺⁺) (A) and non-expressing cells (A2058, EGFR⁻) (B) when matuzumab-SO1861 was titrated on a fixed concentration of 10 pM cetuximab-saporin (cetuximab conjugated to the protein toxin, saporin, with a DAR4) or 10 pM EGFdianthin (recombinant toxin fusion protein) (Legend for FIGS. 6A and 6B is the same and is displayed next to FIG. 6B). Matuzumab recognizes and binds human EGFR at a different epitope compared to cetuximab and EGF, whereas Cetuximab and EGF compete for binding the EGFR receptor.

FIG. 7 (FIG. 7) shows the results of the determination of cetuximab-saporin which was titrated on a fixed concentration of 10 nM and 75 nM matuzumab-SO1861 (Legend for FIGS. 7A and 7B is the same and is displayed next to FIG. 7B).

FIG. 8 (FIG. 8) shows SO1861 titration on a fixed concentration of 10 pM CD71-saporin (DAR4), 10 pM cetuximab-saporin (DAR4), 10 pM matuzumab-dianthin (DAR4), 10 pM pertuzumab-saporin (DAR4), 10 pM or 50 pM pertuzumab-saporin (DAR4) and 50 pM trastuzumab-saporin (DAR4) wherein targeted protein toxin-mediated cell killing on: A) A431 (EGFR⁺⁺/HER2^{+/-}/CD71⁺) and B) A2058 (EGFR⁺/HER2^{+/-}/CD71⁺) was determined.

FIG. 9 (FIG. 9) shows SO1861 titration on a fixed concentration of 10 pM CD71-saporin (DAR4), 10 pM cetuximab-saporin (DAR4), 10 pM matuzumab-dianthin (DAR4), 10 pM pertuzumab-saporin (DAR4), 10 pM or 50 pM pertuzumab-saporin (DAR4) and 50 pM trastuzumab-saporin (DAR4) wherein targeted protein toxin-mediated cell killing on: A) SK-BR-3 cells (HER2⁺⁺/EGFR⁺/CD71⁺) and B) MDA-MB-468 cells (HER2⁻/EGFR⁺⁺/CD71⁺) was determined.

### DETAILED DESCRIPTION

It is a first goal of the present invention to provide an improved ADC or AOC, when for example toxicity, efficacy, therapeutic window, and/or effective dose, and safety for the patient to whom the therapeutic composition comprising such ADC or AOC is administered, is considered. It is a second goal of the present invention to provide an improved method of treatment of a (human) patient suffering from a disease to be treated with an ADC or with an AOC.

It is an objective of the current invention to provide a therapeutic composition or a therapeutic combination of e.g. two therapeutic compositions, comprising an ADC or an AOC, which, when administered to a (human) patient in need thereof, has an improved therapeutic effect or a sufficient effect at a lower dose than the currently required dose for the ADC or the AOC.

At least one of the above objectives is achieved by providing the therapeutic combination of at least two therapeutic compositions or the therapeutic composition of the invention.

The present invention will be described with respect to particular embodiments but the invention is not limited thereto but only by the claims.

A first aspect of the invention relates to a therapeutic combination comprising:

(a) a first pharmaceutical composition comprising a conjugate comprising a first binding molecule comprising a first binding region for binding to a first binding site of a cell-surface molecule and the conjugate comprising at least one saponin covalently bound to said first binding molecule, wherein the saponin is a monodesmosidic triterpene glycoside or a bidesmosidic triterpene glycoside; and
(b) a second pharmaceutical composition comprising a conjugate comprising a second binding molecule different from the first binding molecule, the second binding molecule comprising a second binding region different from the first binding region, the second binding region for binding to a second binding site of said cell-surface molecule different from the first binding site of said cell-surface molecule, and the conjugate comprising an effector molecule covalently bound to said second binding molecule,
the first pharmaceutical composition and the second pharmaceutical composition optionally further comprising a pharmaceutically acceptable excipient and optionally further comprising a pharmaceutically acceptable diluent.

A second aspect of the invention relates to a pharmaceutical composition comprising:
a conjugate comprising a first binding molecule comprising a first binding region for binding to a first binding site of a cell-surface molecule and the conjugate comprising at least one saponin covalently bound to said first binding molecule, wherein the saponin is a triterpenoid saponin of the monodesmosidic type or the bidesmosidic type; and
a conjugate comprising a second binding molecule different from said first binding molecule, the second binding molecule comprising a second binding region different from said first binding region, the second binding region for binding to a second binding site of said cell-surface molecule different from said first binding site of said cell-surface molecule, and the conjugate comprising an effector molecule covalently bound to said second binding molecule,
   and optionally further comprising a pharmaceutically acceptable excipient and optionally further comprising a pharmaceutically acceptable diluent.

In the conjugate comprising the saponin, the conjugate comprises at least one saponin moiety covalently bound to the first binding molecule. A saponin which is part of the conjugate is referred to as ‘saponin’, or ‘saponin moiety’, meaning that the saponin is covalently linked to, here, the first binding molecule.

Pharmaceutically acceptable excipients and pharmaceutically acceptable diluents are well known in the art and suitable excipients and diluents are for example listed in “Remington—The Science and Practice of Pharmacy” (22^{st} Edition, 2013, Lippincott, Williams & Wilkins).

Typical saponins suitable for conjugation are triterpenoid saponins of the bidesmosidic type such as bidesmosidic triterpene glycosides isolated from Quillaja saponaria or isolated and purified from a root extract of Saponaria officinalis, known in the art.

The inventors established that targeting the cell-surface molecule of a target cell with the first binding molecule in the conjugate comprising the saponin and with the second binding molecule in the conjugate comprising the effector molecule, provides for efficient delivery of the two conjugates comprising said first and second binding molecule respectively inside the cell bearing the cell-surface molecule, since the first binding molecule and the second binding molecule bind to different binding sites on the cell-surface molecule. Since both conjugates are deliverable into a target cell based on the targeting of a single type of cell-surface molecule, a cell that exposes only a single (sufficiently) specific cell-surface molecule can now simultaneously be provided intracellularly with the saponin as part of the conjugate comprising the first binding molecule and with the effector molecule as part of the conjugate comprising the second binding molecule. Since binding of the first binding molecule to the cell-surface molecule does not hamper binding of the second binding molecule to the cell-surface molecule, as part of the invention, a dose of both the saponin and the effector molecule can be delivered inside the target cell exposing the single type of cell-surface molecule, using the same cell-surface molecule for entering said cell. Since saponins potentiate effector molecules which exert their biological activity intracellularly, e.g. in the cytosol of e.g. tumor cells, when the saponin and the effector molecule co-localize inside the cell, e.g. in the endosome or lysosome, the therapeutic combination and the pharmaceutical composition provides for an improved therapeutic window when the therapeutic effect of the effector molecule is considered and/or when the potentiating effect of the saponin is considered. The inventors established that saponin is about 100-1000 times more efficiently delivered inside a cell, when a dose of saponin is contacted with the cell wherein the saponin is comprised by a conjugate comprising a binding molecule for a cell-surface molecule of the cell, compared to delivery of free saponin inside said cell. Thus, the effective dose of the conjugate comprising the first binding site for a cell-surface molecule and the saponin is 100-1000 times lower than the effective dose of the free saponin, when the intracellular biological effect of an effector molecule such as a BNA or a (protein) toxin is considered, which effector molecule is simultaneously contacted with the cell together with either the free saponin, or the conjugate comprising the first binding site for a cell-surface molecule. The current invention combines the benefit of targeted delivery of saponin inside a target cell, therewith e.g. improving the therapeutic window when the endosomal escape enhancing activity of the saponin is considered, with the possibility to target a single cell-surface receptor of a target cell with both the conjugate comprising the saponin and the conjugate comprising the effector molecule, simultaneously, such that for example now cells can be efficiently and/or improvingly treated with the effector molecule while such cells only expose a single type of (sufficiently) specific cell-surface molecule that allows for (sufficiently) specific delivery of the effector molecule into the target cell (only). ‘Targeted delivery’ is here to be understood as the delivery of e.g. the saponin inside a cell by specific binding of, here, the first binding molecule to the cell-surface molecule of the target cell, therewith resulting in the endocytosis of the saponin (as part of the conjugate) and the delivery of the saponin in the endosome and/or lysosome.

An embodiment is the therapeutic combination of the invention or the pharmaceutical composition of the invention, wherein the first binding molecule is a first proteinaceous binding molecule or a first non-proteinaceous ligand comprising the first binding region for binding to the first binding site of the cell-surface molecule, and/or wherein the second binding molecule is a second proteinaceous binding molecule or a second non-proteinaceous ligand comprising the second binding region for binding to the second binding site of the cell-surface molecule.

An embodiment is the therapeutic combination of the invention or the pharmaceutical composition of the invention, wherein the first binding molecule is a first proteinaceous binding molecule and wherein the saponin is covalently bound to an amino acid residue of the first binding molecule, preferably via a linker.

Typically, for the therapeutic combination of the invention or the pharmaceutical composition of the invention, the first binding site is a first epitope of said cell-surface molecule such as a cell-surface receptor and wherein the second binding site is a second epitope of said, same, cell-surface molecule, wherein the second epitope is different from the first epitope.

An embodiment is the therapeutic combination of the invention or the pharmaceutical composition of the invention, wherein the saponin is a bidesmosidic triterpene saponin.

An embodiment is the therapeutic combination of the invention or the pharmaceutical composition of the invention, wherein the cell-surface molecule is a tumor-cell surface molecule, preferably a tumor cell-specific cell-surface molecule, such as a cell-surface receptor. “Specific” in the context of presence of a cell-surface molecule on a cell has its regular scientific meaning and refers to the presence of the molecule on the cell whereas the same molecule is absent on other cells or cell types, or is present to a lower extent (less copies of the molecule) on the surface of cells different than the cells referred to bearing the cell-specific molecule. Tumor cells may have truly tumor-cell specific cell-surface molecules such as receptors, or may express a cell-surface molecule to a higher extent, and may have more copies of the specific cell-surface molecule on its surface, compared to non-tumor cells, such as healthy cells of the same type or in the organ bearing the tumor comprising the tumor cells.

An embodiment is the therapeutic combination of the invention or the pharmaceutical composition of the invention, wherein the first binding region of the first binding molecule comprises or consists of a ligand for binding to the first binding site of the cell-surface molecule such as EGF or a cytokine, or wherein the first binding region of the first binding molecule comprises or consists of an immunoglobulin or at least one binding fragment or binding domain of said immunoglobulin comprising the first binding region for binding to the first binding site of the cell-surface molecule, and/or wherein the second binding region of the second binding molecule comprises or consists of a ligand for binding to the second binding site of the cell-surface molecule such as EGF or a cytokine, or wherein the second binding region of the second binding molecule comprises or consists of an immunoglobulin or at least one binding fragment or binding domain of said immunoglobulin comprising the second binding region for binding to the second binding site of the cell-surface molecule, wherein the immunoglobulin is preferably any one or more of an antibody such as a monoclonal antibody, preferably a human antibody, an IgG, a molecule comprising or consisting of a single-domain antibody, at least one V_{HH} domain such as a camelid V_{H}, or at least one V_{H} domain, such as from human origin, a variable heavy chain new antigen receptor (V_{NAR}) domain, a Fab, an scFv, an Fv, a dAb, an F(ab)2, a Fcab fragment.

An embodiment is the therapeutic combination of the invention or the pharmaceutical composition of the invention, wherein the first binding region of the first binding molecule comprises or consists of a monoclonal antibody, a single-domain antibody, at least one V_{HH} domain, at least one V_{H} domain, a variable heavy chain new antigen receptor (V_{NAR}) domain, a Fab, an scFv, an Fv, a dAb, an F(ab)₂, or a Fcab fragment, preferably a monoclonal antibody or a single-domain antibody, such as at least one V_{HH} domain, and/or wherein the second binding region of the second binding molecule comprises or consists of a monoclonal antibody, a single-domain antibody, at least one V_{HH} domain, at least one V_{H} domain, a variable heavy chain new antigen receptor (V_{NAR}) domain, a Fab, an scFv, an Fv, a dAb, an F(ab)₂, or a Fcab fragment, preferably a monoclonal antibody or a single-domain antibody, such as at least one V_{HH} domain. For example, the first binding region is matuzumab and the second binding region is V_{HH} 7D12 with amino-acid sequence of SEQ ID NO: 1, or vice versa, or the first binding region is cetuximab and the second binding region is V_{HH} 9G8 with amino-acid sequence of SEQ ID NO: 2, or vice versa. Typically, the first binding region is matuzumab and the second binding region is cetuximab, or vice versa. Typically, the first binding region is trastuzumab and the second binding region is pertuzumab, or vice versa.

An embodiment is the therapeutic combination of the invention or the pharmaceutical composition of the invention, wherein the at least one binding fragment or binding domain of said immunoglobulin comprising the first binding region for binding to the first binding site of the cell-surface molecule and/or the at least one binding fragment or binding domain of said immunoglobulin comprising the second binding region for binding to the second binding site of the cell-surface molecule is a single-domain antibody, preferably at least one V_{HH} domain.

Typically for the the therapeutic combination of the invention or the pharmaceutical composition of the invention, the first binding region and the second binding region are selected to simultaneously bind the same cell-surface molecule at the first binding site and at the second binding site. That is to say, binding of the first binding region to the first binding site (first epitope) does not hinder or exclude or prevent or block or compete for binding of the second binding region to the second binding site (second epitope) of the same cell surface molecule such as a cell receptor exposed at the cell surface.

Preferred is the therapeutic combination of the invention or the pharmaceutical composition of the invention, wherein the first binding region is selected to bind to the first binding site of the cell-surface molecule without competing for the binding of the second binding region to the second binding site of the same cell-surface molecule, and wherein the second binding region is selected to bind to the second binding site of the cell-surface molecule without competing for the binding of the first binding region to the first binding site of the same cell-surface molecule.

An embodiment is the therapeutic combination of the invention or the pharmaceutical composition of the invention, wherein the at least one saponin is a bidesmosidic triterpene saponin (glycoside) belonging to the type of a 12,13-dehydrooleanane with an aldehyde function in position C₂₃ (of the (triterpene) aglycone core structure of the saponin), the saponin comprising a first saccharide chain at the C₃beta-OH group (of the (triterpene) aglycone core structure) of the saponin, the first saccharide chain optionally comprising a glucuronic acid moiety, and the saponin comprising a second saccharide chain linked to C₂₈ (of the (triterpene) aglycone core structure) of the saponin and comprising or consisting of a monosaccharide or a linear or branched oligosaccharide wherein optionally at least one saccharide moiety of the second saccharide chain comprises at least one acetyl group, for example 1, 2, 3 or 4 acetyl groups, and preferably a single acetyl group.

An embodiment is the therapeutic combination of the invention or the pharmaceutical composition of the invention, wherein the at least one saponin is a saponin isolated from any one or more of a Gypsophila species, a Saponaria species, an Agrostemma species and a Quillaja species such as Quillaja saponaria. Typically, saponins suitable for conjugation are isolated from extracts from the bark of Quillaja saponaria or are isolated from a root extract of Saponaria officinalis. Thus, according to the invention, saponins in the conjugates of the invention are for example naturally occurring saponins, although triterpene glycosides with similar structural features with regard to the aglycone core structure and the (poly-/mono-)saccharide structures can also be synthetic saponins. Of course, for the conjugates, naturally occurring saponins can also be implied which are chemically synthesized, if suitable and available.

An embodiment is the therapeutic combination of the invention or the pharmaceutical composition of the invention, wherein the at least one saponin comprises an aglycone core structure selected from any one or more of (the aglycone(s) (core structures)):

2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
digitogenin;
3,16,28-trihydroxy oleanan-12-en;
gypsogenic acid; and
a derivative thereof,
preferably, the aglycone core structure is selected from quillaic acid and gypsogenin or a derivative thereof, most preferably the aglycone core structure is quillaic acid or a derivative thereof. The inventors found that saponins comprising an aglycone which comprises an aldehyde group in the triterpene structure are particularly suitable for incorporation in the conjugate of the invention. Without wishing to be bound by any theory, the presence of the aldehyde group in the saponin may contribute to endosomal escape enhancing activity of the saponin, when the delivery of an effector molecule from outside a (mammalian) cell to inside said cell, in the endosome of said cell, and subsequently out of the cell endosome and into the cytosol of said cell, is considered.

An embodiment is the therapeutic combination of the invention or the pharmaceutical composition of the invention, wherein the at least one saponin comprises a first saccharide chain bound to its aglycone core structure, selected from:

### GlcA-,

### Glc-,

### Gal-,

### Rha-(1→2)-Ara-,

### Gal-(1→2)-[Xyl-(1→3)]-GlcA-,

### Glc-(1→2)-[Glc-(1→4)]-GlcA-,

### Glc-(1→2)-Ara-(1→3)-[Gal-(1→2)]-GlcA-,

### Xyl-(1→2)-Ara-(1→3)-[Gal-(1→2)]-GlcA-,

### Glc-(1→3)-Gal-(1→2)-[Xyl-(1→3)]-Glc-(1→4)-Gal-,

### Rha-(1→2)-Gal-(1→3)-[Glc-(1→2)]-GlcA-,

### Ara-(1→4)-Rha-(1→2)-Glc-(1→2)-Rha-(1→2)-GlcA-,

### Ara-(1→4)-Fuc-(1→2)-Glc-(1→2)-Rha-(1→2)-GlcA-,

### Ara-(1→4)-Rha-(1→2)-Gal-(1→2)-Rha-(1→2)-GlcA-,

### Ara-(1→4)-Fuc-(1→2)-Gal-(1→2)-Rha-(1→2)-GlcA-,

### Ara-(1→4)-Rha-(1→2)-Glc-(1→2)-Fuc-(1→2)-GlcA-,

### Ara-(1→4)-Fuc-(1→2)-Glc-(1→2)-Fuc-(1→2)-GlcA-,

### Ara-(1→4)-Rha-(1→2)-Gal-(1→2)-Fuc-(1→2)-GlcA-,

### Ara-(1→4)-Fuc-(1→2)-Gal-(1→2)-Fuc-(1→2)-GlcA-,

### Xyl-(1→4)-Rha-(1→2)-Glc-(1→2)-Rha-(1→2)-GlcA-,

### Xyl-(1→4)-Fuc-(1→2)-Glc-(1→2)-Rha-(1→2)-GlcA-,

### Xyl-(1→4)-Rha-(1→2)-Gal-(1→2)-Rha-(1→2)-GlcA-,

### Xyl-(1→4)-Fuc-(1→2)-Gal-(1→2)-Rha-(1→2)-GlcA-,

### Xyl-(1→4)-Rha-(1→2)-Glc-(1→2)-Fuc-(1→2)-GlcA-,

### Xyl-(1→4)-Fuc-(1→2)-Glc-(1→2)-Fuc-(1→2)-GlcA-,

### Xyl-(1→4)-Rha-(1→2)-Gal-(1→2)-Fuc-(1→2)-GlcA-,

### Xyl-(1→4)-Fuc-(1→2)-Gal-(1→2)-Fuc-(1→2)-GlcA-, and

any derivative thereof,
and/or wherein the at least one saponin optionally comprises a second saccharide chain bound to its aglycone core structure, selected from:

### Glc-,

### Gal-,

### Rha-(1→2)-[Xyl-(1→4)]-Rha-,

### Rha-(1→2)-[Ara-(1→3)-Xyl-(1→4)]-Rha-,

### Ara-,

### Xyl-,

Xyl-(1→4)-Rha-(1→2)-[R1-(→4)]-Fuc- wherein R1 is 4E-Methoxycinnamic acid,
Xyl-(1→4)-Rha-(1→2)-[R2-(→4)]-Fuc- wherein R2 is 4Z-Methoxycinnamic acid,

### Xyl-(1→4)-[Gal-(1→3)]-Rha-(1→2)-4-OAc-Fuc-,

### Xyl-(1→4)-[Glc-(1→3)]-Rha-(1→2)-3,4-di-OAc-Fuc-,

Xyl-(1→4)-[Glc-(1→3)]-Rha-(1→2)-[R3-(→4)]-3-OAc-Fuc- wherein R3 is 4E-Methoxycinnamic acid,

### Glc-(1→3)-Xyl-(1→4)-[Glc-(1→3)]-Rha-(1→2)-4-OAc-Fuc-,

### Glc-(1→3)-Xyl-(1→4)-Rha-(1→2)-4-OAc-Fuc-,

### (Ara- or Xyl-)(1→3)-(Ara- or Xyl-)(1→4)-(Rha- or Fuc-)(1→2)-[4-OAc-(Rha- or Fuc-)(1→4)]-(Rha- or Fuc-),

### Xyl-(1→3)-Xyl-(1→4)-Rha-(1→2)-[Qui-(1→4)]-Fuc-,

### Api-(1→3)-Xyl-(1→4)-[Glc-(1→3)]-Rha-(1→2)-Fuc-,

### Xyl-(1→4)-[Gal-(1→3)]-Rha-(1→2)-Fuc-,

### Xyl-(1→4)-[Glc-(1→3)]-Rha-(1→2)-Fuc-,

### Ara/Xyl-(1→4)-Rha/Fuc-(1→4)-[Glc/Gal-(1→2)]-Fuc-,

Api-(1→3)-Xyl-(1→4)-[Glc-(1→3)]-Rha-(1→2)-[R4-(→4)]-Fuc- wherein R4 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R5-(→4)]-Fuc- wherein R5 is 5-O-[5-O-Ara/Api-3,5-dihydroxy-6-methyl-octanoyl]-3,5-dihydroxy-6-methyl-octanoic acid),

### Api-(1→3)-Xyl-(1→4)-Rha-(1→2)-[Rha-(1→3)]-4-OAc-Fuc-,

### Api-(1→3)-Xyl-(1→4)-[Glc-(1→3)]-Rha-(1→2)-[Rha-(1→3)]-4-OAc-Fuc-,

### 6-OAc-Glc-(1→3)-Xyl-(1→4)-Rha-(1→2)-[3-OAc-Rha-(1→3)]-Fuc-,

### Glc-(1→3)-Xyl-(1→4)-Rha-(1→2)-[3-OAc-Rha-(1→3)]-Fuc-,

### Xyl-(1→3)-Xyl-(1→4)-Rha-(1→2)-[Qui-(1→4)]-Fuc-,

### Glc-(1→3)-[Xyl-(1→4)]-Rha-(1→2)-[Qui-(1→4)]-Fuc-,

### Glc-(1→3)-Xyl-(1→4)-Rha-(1→2)-[Xyl-(1→3)-4-OAc-Qui-(1→4)]-Fuc-,

### Xyl-(1→3)-Xyl-(1→4)-Rha-(1→2)-[3,4-di-OAc-Qui-(1→4)]-Fuc-,

### Glc-(1→3)-[Xyl-(1→4)]-Rha-(1→2)-Fuc-,

### 6-OAc-Glc-(1→3)-[Xyl-(1→4)]-Rha-(1→2)-Fuc-,

### Glc-(1→3)-[Xyl-(1→3)-Xyl-(1→4)]-Rha-(1→2)-Fuc-,

### Xyl-(1→3)-Xyl-(1→4)-Rha-(1→2)-[Xyl-(1→3)-4-OAc-Qui-(1→4)]-Fuc-,

### Api/Xyl-(1→3)-Xyl-(1→4)-[Glc-(1→3)]-Rha-(1→2)-[Rha-(1→3)]-4OAc-Fuc-,

### Api-(1→3)-Xyl-(1→4)-[Glc-(1→3)]-Rha-(1→2)-[Rha-(1→3)]-4OAc-Fuc-,

Api/Xyl-(1→3)-Xyl-(1→4)-[Glc-(1→3)]-Rha-(1→2)-[R6-(→4)]-Fuc- wherein R6 is 5-O-[5-O-Rha-(1→2)-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R7-(→4)]-Fuc- wherein R7 is 5-O-[5-O-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R8-(→4)]-Fuc- wherein R8 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R9-(→4)]-Fuc- wherein R9 is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R10-(→4)]-Fuc- wherein R10 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R11-(→3)]-Fuc- wherein R11 is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R12-(→3)]-Fuc- wherein R12 is 5-O-[5-O-Ara/Api-3,5-dihydroxy-6-methyl-octanoyl]-3,5-dihydroxy-6-methyl-octanoic acid),

### Glc-(1→3)-[Glc-(1→6)]-Gal-, and

a derivative thereof,
preferably the at least one saponin comprises such a first saccharide chain and comprises such a second saccharide chain bound to the aglycone core structure of the saponin, i.e. such an aglycone as listed here above, preferably quillaic acid or gypsogenin, having an aldehyde group at position C₂₃ of the aglycone. The first glycan is bound to the C₃ atom of the aglycone of the saponin, the second glycan is bound to the C₂₈ atom of the aglycone of the saponin.

An embodiment is the therapeutic combination of the invention or the pharmaceutical composition of the invention, wherein the at least one saponin is any one or more of: Quillaja bark saponin, dipsacoside B, saikosaponin A, saikosaponin D, macranthoidin A, esculentoside A, phytolaccagenin, aescinate, AS6.2, NP-005236, AMA-1, AMR, alpha-Hederin, NP-012672, NP-017777, NP-017778, NP-017774, NP-018110, NP-017772, NP-018109, NP-017888, NP-017889, NP-018108, SA1641, AE X55, NP-017674, NP-017810, AG1, NP-003881, NP-017676, NP-017677, NP-017706, NP-017705, NP-017773, NP-017775, SA1657, AG2, SO1861, GE1741, S01542, S01584, S01658, S01674, S01832, SO1862, S01904, QS-7, QS1861, QS-7 api, QS1862, QS-17, QS-18, QS-21 A-apio, QS-21 A-xylo, QS-21 B-apio, QS-21 B-xylo, beta-Aescin, Aescin Ia, Teaseed saponin I, Teaseedsaponin J, Assamsaponin F, Digitonin, Primula acid 1 and AS64R, or a saponin derivative based thereon, or any of their stereoisomers and/or any combinations thereof, preferably any one or more of QS-21, a QS-21 derivative, SO1861, a SO1861 derivative, SA1641, a SA1641 derivative, GE1741 and a GE1741 derivative, more preferably QS-21, a QS-21 derivative, SO1861 or a SO1861 derivative, most preferably SO1861 or a SO1861 derivative.

Saponins suitable for incorporation in the conjugates of the invention comprising the saponin are typically the saponins listed in Table A1. These saponins have either been shown to enhance the endosomal escape of effector molecules once taken up by a cell such as taken up by endocytosis, when the saponins are contacted with such cells that are exposed to such effector molecules; or these listed saponins have molecular structures (highly) reminiscent to the saponins for which the endosomal escape enhancing activity has been established.

An embodiment is the therapeutic combination of the invention or the pharmaceutical composition of the invention, wherein the (saponin or) saponin moiety or the (saponin derivative or) saponin derivative moiety in the first conjugate comprises the first saccharide chain and comprises the second saccharide chain, wherein the first saccharide chain comprises more than one saccharide moiety and the second saccharide chain comprises more than one saccharide moiety, and wherein the aglycone core structure of the saponin is, or is a derivative of, quillaic acid or gypsogenin, wherein one, two or three, preferably one or two, of:
i. an aldehyde group in the aglycone core structure of the saponin has been derivatised,
ii. a carboxyl group of a glucuronic acid moiety in the first saccharide chain has been derivatised, and
iii. at least one acetoxy (Me(CO)O—) group in the second saccharide chain has been derivatised.

An embodiment is the therapeutic combination of the invention or the pharmaceutical composition of the invention, wherein the saponin moiety or the saponin derivative moiety in the first conjugate comprises:
i. an aglycone core structure comprising an aldehyde group which has been derivatised by:
   reduction to an alcohol;
   transformation into a hydrazone bond through reaction with N-ε-maleimidocaproic acid hydrazide (EMCH), wherein the maleimide group of the EMCH is optionally derivatised by formation of a thioether bond with mercaptoethanol;
   transformation into a hydrazone bond through reaction with N-[ß-maleimidopropionic acid] hydrazide (BMPH), wherein the maleimide group of the BMPH is optionally derivatised by formation of a thioether bond with mercaptoethanol; or
   transformation into a hydrazone bond through reaction with N-[κ-maleimidoundecanoic acid] hydrazide (KMUH), wherein the maleimide group of the KMUH is optionally derivatised by formation of a thioether bond with mercaptoethanol;
ii. a first saccharide chain comprising a carboxyl group, preferably a carboxyl group of a glucuronic acid moiety, which has been derivatised by transformation into an amide bond through reaction with 2-amino-2-methyl-1,3-propanediol (AMPD) or N-(2-aminoethyl)maleimide (AEM);
iii. a second saccharide chain comprising an acetoxy group (Me(CO)O—) which has been derivatised by transformation into a hydroxyl group (HO−) by deacetylation; or
iv. any combination of two or three, preferably two, derivatisations of derivatisations i., ii. and iii.

An embodiment is the therapeutic combination of the invention or the pharmaceutical composition of the invention, wherein the at least one saponin is any one or more of: SO1861, SA1657, GE1741, SA1641, QS-21, QS-21A, QS-21 A-api, QS-21 A-xyl, QS-21 B, QS-21 B-api, QS-21 B-xyl, QS-7-xyl, QS-7-api, QS-17-api, QS-17-xyl, QS1861, QS1862, Quillaja saponin, Saponinum album, QS-18, Quil-A, Gyp1, gypsoside A, AG1, AG2, S01542, S01584, S01658, S01674, S01832, or a saponin derivative thereof, or a stereoisomer thereof and/or any combination thereof, preferably any one or more of QS-21 or a QS-21 derivative, SO1861 or a SO1861 derivative, SA1641 or a SA1641 derivative and GE1741 or a GE1741 derivative, more preferably a QS-21 derivative or a SO1861 derivative, most preferably SO1861 or a SO1861 derivative. Typically, such saponins enhance the endosomal escape of an effector molecule such as a BNA or a (protein) toxin, when a cell is contacted with the pharmaceutical combination or the pharmaceutical composition comprising the conjugate comprising the saponin and the conjugate comprising the effector molecule, of the invention.

An embodiment is the therapeutic combination of the invention or the pharmaceutical composition of the invention, wherein the at least one saponin is a bidesmosidic triterpene glycoside belonging to the type of a 12,13-dehydrooleanane with an aldehyde function in position C₂₃ of the aglycone core structure of the saponin, wherein the saponin is covalently bound to the first binding molecule. Preferably, the saponin is covalently bound to an amino-acid residue of the first binding molecule, via an aldehyde function in the saponin, preferably said aldehyde function in position C₂₃ of the aglycone core structure. Said binding of the saponin to the first binding molecule is preferably via at least one linker, and/or via at least one cleavable linker, wherein the amino-acid residue of the first binding molecule preferably is selected from cysteine and lysine.

An embodiment is the therapeutic combination of the invention or the pharmaceutical composition of the invention, wherein the aldehyde function in position C₂₃ of the aglycone core structure of the at least one saponin is covalently bound to linker EMCH, which linker is covalently bound via a thio-ether bond to a sulfhydryl group in the first binding molecule, such as a sulfhydryl group of a cysteine. The advantage of such EMCH linker is the decomposition of the conjugate comprising the saponin and the first binding molecule under influence of the pH conditions in the endosome of (mammalian) cells, once such a conjugate is transferred from outside a cell to inside said endosome of said cell. Without wishing to be bound by any theory, under the acidic conditions in the endosome, the saponin cleaves off from the conjugate comprising the saponin linked through the linker to the binding molecule, which freeing of the saponin results in occurrence of the aldehyde group in the aglycone of the saponin, which aldehyde group contributes to the endosomal escape enhancing activity when the endosomal escape of the effector molecule (of the conjugate comprising the effector molecule) is considered.

An embodiment is the therapeutic combination of the invention or the pharmaceutical composition of the invention, wherein the at least one saponin is a bidesmosidic triterpene glycoside belonging to the type of a 12,13-dehydrooleanane with an aldehyde function in position C₂₃ of the aglycone core structure of the saponin and comprising a glucuronic acid unit in a first saccharide chain at the C₃beta-OH group of the aglycone core structure of the saponin, wherein the saponin is covalently bound to an amino-acid residue of the first binding molecule via the carboxyl group of the glucuronic acid unit in the first saccharide chain, preferably via a linker, wherein the amino-acid residue preferably is selected from cysteine and lysine. An advantage of coupling the saponin to the first binding molecule in the conjugate comprising the saponin, via the carboxyl group, is the availability of the free aldehyde group in the saponin aglycone. Again, without wishing to be bound by any theory the free aldehyde group contributes to the endosomal escape enhancing effect seen when an effector molecule is contacted with a cell, e.g. as part of the conjugate comprising the effector molecule, together with the conjugate comprising the saponin.

An embodiment is the therapeutic combination of the invention or the pharmaceutical composition of the invention, wherein the at least one saponin comprises a glucuronic acid unit in its first saccharide chain at the C₃beta-OH group of the aglycone core structure of the at least one saponin, which glucuronic acid unit is covalently bound to linker 1-[Bis(dimethylamino)methylene]-1H-1,2,3-triazolo[4,5-b]pyridinium 3-oxid hexafluorophosphate (HATU), which linker is preferably covalently bound via an amide bond to an amine group in the first binding molecule, such as an amine group of a lysine or an N-terminus of the first binding molecule if the first binding molecule is a first proteinaceous binding molecule. Again, coupling of a saponin to the first binding molecule, such as a peptide or a protein, e.g. an antibody, a ligand such as EGF, via the carboxyl group of a glucuronic acid unit in the saccharide chain of the saponin, provides for a free aldehyde group in the aglycone of the saponin. HATU is an example of a linker suitable for coupling the saponin to the proteinaceous molecule. The skilled person will appreciate that further linkers can be equally suitable for the purpose of linking the saponin via a carboxyl group in its glycan to a first binding molecule. Suitable linkers are for example outlined in “Bioconjugate Techniques” (G. T. Hermanson, 3^{rd} Edition, 2013, Elsevier Academic Press).

An embodiment is the therapeutic combination of the invention or the pharmaceutical composition of the invention, wherein the cell-surface molecule is a cell-surface receptor, preferably a tumor-cell specific cell-surface receptor, more preferably a receptor selected from any one or more of: CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, PSMA, CanAg, integrin-alphaV, CA6, CD33, mesothelin, Cripto, CD3, CD30, CD239, CD70, CD123, CD352, DLL3, CD25, ephrinA4, MUC1, Trop2, CEACAM5, CEACAM6, HER3, CD74, PTK7, Notch3, FGF2, C4.4A, FLT3, CD38, FGFR3, CD7, PD-L1, CTLA4, CD52, PDGFRA, VEGFR1, VEGFR2, most preferably selected from: HER2, CD71 and EGFR. It is preferred that the first binding molecule and the second binding molecule is capable of binding to a proteinaceous cell-surface molecule such as a surface receptor, i.e. the same cell-surface molecule. In particular, receptors are preferred as target for binding of the conjugate comprising the saponin and the conjugate comprising the effector molecule, which receptors are preferably highly expressed on the target cell such as a tumor cell, or which are even uniquely expressed on the target cell such as a tumor cell. HER2, CD71 and EGFR are receptors expressed at tumor cells which can suitably be targeted by the conjugate of the invention comprising the saponin and the conjugate of the invention comprising the effector molecule.

An embodiment is the therapeutic combination of the invention or the pharmaceutical composition of the invention, wherein the first binding region of the first binding molecule and the second binding region of the second binding molecule comprise or consist of an antibody or a cell-surface molecule binding fragment thereof or cell-surface molecule binding domain(s) thereof and/or comprise or consist of a ligand for binding to the cell-surface molecule, preferably selected from: an anti-CD71 monoclonal antibody such as IgG type OKT-9 and a second anti-CD71antibody; an anti-HER2 monoclonal antibody such as trastuzumab (Herceptin), pertuzumab and a third anti-HER2 monoclonal antibody; an anti-CD20 monoclonal antibody such as rituximab, ofatumumab, tositumomab, obinutuzumab ibritumomab and a fifth anti-CD20 monoclonal antibody; an anti-CA125 monoclonal antibody such as oregovomab and a second anti-CA125 monoclonal antibody; an anti-EpCAM (17-1A) monoclonal antibody such as edrecolomab and a second anti-EpCAM (17-1A) monoclonal antibody; an anti-EGFR monoclonal antibody such as cetuximab, matuzumab, panitumumab, nimotuzumab and a fifth anti-EGFR monoclonal antibody or EGF; an anti-CD30 monoclonal antibody such as brentuximab and a second anti-CD30 antibody; an anti-CD33 monoclonal antibody such as gemtuzumab, huMy9-6 and a third anti-CD33 monoclonal antibody; an anti-vascular integrin alpha-v beta-3 monoclonal antibody such as etaracizumab and a second anti-vascular integrin alpha-v beta-3 antibody; an anti-CD52 monoclonal antibody such as alemtuzumab and a second anti-CD52 antibody; an anti-CD22 monoclonal antibody such as epratuzumab, pinatuzumab, binding fragment (Fv) of anti-CD22 antibody moxetumomab, humanized monoclonal antibody inotuzumab and a fifth anti-CD22 monoclonal antibody; an anti-CEA monoclonal antibody such as labetuzumab and a second anti-CEA monoclonal antibody; an anti-CD44v6 monoclonal antibody such as bivatuzumab and a second anti-CD44v6 monoclonal antibody; an anti-FAP monoclonal antibody such as sibrotuzumab and a second anti-FAB monoclonal antibody; an anti-CD19 monoclonal antibody such as huB4 and a second anti-CD19 monoclonal antibody; an anti-CanAg monoclonal antibody such as huC242 and a second anti-CanAg monoclonal antibody; an anti-CD56 monoclonal antibody such as huN901 and a second anti-CD56 monoclonal antibody; an anti-CD38 monoclonal antibody such as daratumumab, OKT-10 anti-CD38 monoclonal antibody and a third anti-CD38 monoclonal antibody; an anti-CA6 monoclonal antibody such as DS6 and a second anti-CA6 monoclonal antibody; an anti-IGF-1R monoclonal antibody such as cixutumumab, 3B7 and a third anti-CA6 monoclonal antibody; an anti-integrin monoclonal antibody such as CNTO 95 and a second anti-integrin monoclonal antibody; an anti-syndecan-1 monoclonal antibody such as B-B4 and a second anti-syndecan-1 monoclonal antibody; an anti-CD79b monoclonal antibody such as polatuzumab and a second anti-CD79b monoclonal antibody, preferably any one of: trastuzumab and pertuzumab; cetuximab and matuzumab; matuzumab and V_{HH} 7D12 with amino-acid sequence of SEQ ID NO: 1; cetuximab and V_{HH} 9G8 with amino-acid sequence of SEQ ID NO: 2; and EGF and matuzumab, with the proviso that the first binding region and the second binding region are different and with the proviso that the first binding site and the second binding site are different.

As further detailed in the Examples section, the inventors established that for example EGFR and HER2 can be targeted with a first and second binding molecule capable of binding to different binding sites on the HER or on the EGFR. Such first and second binding molecule, which are different, are in certain embodiments part of the conjugate comprising the saponin and the conjugate comprising the effector molecule, respectively.

An embodiment is the therapeutic combination of the invention or the pharmaceutical composition of the invention, wherein the first binding region of the first binding molecule is capable of binding to the first binding site of the cell-surface receptor and the second binding region of the second binding molecule is capable of binding to the second binding site of the cell-surface receptor, simultaneously. This way, a target cell that has the cell-surface receptor exposed on its surface is the binding target for both the conjugate comprising the first binding molecule and the conjugate comprising the second binding molecule at the same time. That is to say, the two conjugates can bind to the target cell together, and even to the very same cell-surface receptor molecule, wherein binding of the first binding molecule to the cell-surface receptor does not exclude the binding of the second binding molecule to the cell-surface receptor, and vice versa. Preferably, in the pharmaceutical combination of the invention or the pharmaceutical composition of the invention, the conjugate comprising the first binding molecule and the conjugate comprising the second molecule can bind to the same cell-surface molecule, simultaneously.

Targeting the same cell-surface molecule with the first binding molecule and with the second binding molecule has several advantages. First, when the cell-surface molecule such as a (tumor-cell specific) receptor, is expressed at the cell surface to a relatively low extent (relatively few copies of the receptor are present on the cell surface), there can still be sufficient binding sites available for the first binding molecule and the second binding molecule, since these binding molecules are capable of binding to the same receptor molecule, without mutually excluding each others binding. This way, also low-expressing cells when the target cell-surface molecule is considered, can efficiently be targeted by the conjugates comprising the first and second binding molecule, such that the saponin and the effector molecule can be transferred to inside the cell together. Second, when a target cell such as a tumor cell only exposes a single type of cell-surface molecule such as a (tumor-cell specific) receptor, that is sufficiently specific for the target (tumor) cell, this single (tumor-)cell specific cell-surface molecule can still be used for targeting by both the conjugate comprising the first binding molecule and the saponin and the conjugate comprising the second binding molecule and the effector molecule, such that both conjugates can enter the cell together and the effector molecule can exert its biological activity inside the cell by reaching the target molecule of the effector molecule inside the cell. Targeting such as single (sufficiently) specific cell-surface molecule with both the first binding molecule and the second binding molecule avoids the targeting of a second cell-surface molecule on the same target cell by e.g. a further binding molecule in a further conjugate comprising the effector molecule, such further binding molecule being different from both the first and second binding molecule, wherein the second surface molecule is less or not specific for the target cell. Thus, targeting the (single) specific cell-surface molecule of a target cell for the conjugate comprising the first binding molecule and the saponin and for the conjugate comprising the second binding molecule and the effector molecule, provides for a more specific targeting of the target cell, for example when the target cell does not comprise a second cell-surface molecule that could provide sufficiently specific binding of a further binding molecule, when the targeting of the target cell is considered. The more specific a target cell is targeted by the conjugates comprising the first and second binding molecules for binding to the same cell-surface molecule, the less risk for off-target cell binding is apparent. A single cell-surface molecule on a target cell that is sufficiently specific for such target cell is enough for specific delivery of both the saponin and the effector molecule inside the target cell under influence of the concomitant binding of the conjugate comprising the first binding molecule and the saponin and binding of the conjugate comprising the second binding molecule and the effector molecule to the same cell-surface molecule.

An embodiment is the therapeutic combination of the invention or the pharmaceutical composition of the invention, wherein the first binding region of the first binding molecule is capable of binding to the first binding site of the cell-surface receptor without blocking the capacity of the second binding region of the second binding molecule to bind to the second binding site of the cell-surface receptor simultaneously, and/or wherein the second binding region of the second binding molecule is capable of binding to the second binding site of the cell-surface receptor without blocking the capacity of the first binding region of the first binding molecule to bind to the first binding site of the cell-surface receptor simultaneously. This way, for example, even cells that express the cell-surface molecule to a relatively low extent at their surface can still be efficiently targeted and bound by the two conjugates comprising the first and second binding molecule. Moreover, one of the many benefits of the combination of the conjugate comprising the first binding molecule and the saponin and the second conjugate comprising the second binding molecule and the effector molecule, wherein the first and second binding molecule bind to the same cell-surface molecule of a target cell but to a different binding site on said cell-surface molecule, is the avoidance of competitive binding. That is to say, if the first and second binding molecule would be the same, or would bind to the same or (highly) overlapping binding site on the same cell-surface molecule, binding of the conjugate comprising the first binding site would e.g. prevent, exclude, hinder, or disrupt the binding of the conjugate comprising the second binding molecule, and vice versa. This may hamper or limit or prevent efficient entrance of the conjugates together inside the target cell, for example at least when a dose sufficient for the desired effect of the effector molecule is considered and/or of the saponin is considered. When the cell-surface molecule is not sufficiently high expressed on the target cell, targeting the very same or overlapping binding site on the cell-surface molecule can even prevent the beneficial additive or synergistic effect seen when the target cell is targeted by the conjugates of the invention, which bind to a first and second binding site on the cell-surface receptor, without hampering the mutual binding of each other, achieved with the conjugates of the invention.

An embodiment is the therapeutic combination of the invention or the pharmaceutical composition of the invention, wherein the effector molecule comprises or consists of at least one of a small molecule such as a drug molecule, a toxin such as a protein toxin, an oligonucleotide such as a BNA, a xeno nucleic acid or an siRNA, an enzyme, a peptide, a protein, or any combination thereof, preferably, the effector molecule is a toxin, an enzyme or an oligonucleotide, more preferably, the effector molecule comprises or consists of at least one of an oligonucleotide, a nucleic acid and a xeno nucleic acid. It is part of the invention that the effector molecule can be any molecule selected for and capable of exerting a biological effect inside a cell once an intracellular target molecule (binding partner) of the effector molecule is bound inside said cell. Such effector molecules are well known in the art, for example in the field of ADC selection and design and in the field of AOC, enzyme restoration or replacement therapy, gene therapy (knocking-in, knocking-out), etc. It is part of the invention that any effector molecule known in the art for being capable of exerting a desired and selected biological effect inside a cell, once the effector molecule is delivered inside said cell, in particular in the cytosol of said cell, is suitable for incorporation in the conjugate comprising the second binding molecule and the effector molecule. Thus, suitable are for example effector molecules for which the target molecule inside a target cell that exposes the cell-surface molecule to which the first and second binding molecule can bind, is present. Thus, suitable are for example effector molecules for which it is established that they can exert a desired biological effect inside a target cell that exposes the cell-surface molecule to which the first and second binding molecule can bind.

An embodiment is the therapeutic combination of the invention or the pharmaceutical composition of the invention, wherein the effector molecule is selected from any one or more of a vector, a gene, a cell suicide inducing transgene, deoxyribonucleic acid (DNA), ribonucleic acid (RNA), anti-sense oligonucleotide (ASO, AON), short interfering RNA (siRNA), anti-microRNA (anti-miRNA), DNA aptamer, RNA aptamer, mRNA, mini-circle DNA, peptide nucleic acid (PNA), phosphoramidate morpholino oligomer (PMO), locked nucleic acid (LNA), bridged nucleic acid (BNA), 2′-deoxy-2′-fluoroarabino nucleic acid (FANA), 2′-O-methoxyethyl-RNA (MOE), 2′-O,4′-aminoethylene bridged nucleic acid, 3′-fluoro hexitol nucleic acid (FHNA), a plasmid, glycol nucleic acid (GNA) and threose nucleic acid (TNA), or a derivative thereof, more preferably a BNA, for example a BNA for silencing HSP27 protein expression or a BNA for silencing apolipoprotein B expression. Under influence of the conjugate comprising the saponin, such an oligonucleotide is efficiently delivered in the cytosol of the target cell bearing the cell-surface molecule for binding of the first and second binding molecule, either directly or via the endosomal escape after endocytosis. Under influence of the targeted saponin the oligonucleotide is improvingly delivered from the endosome (or lysosome) into the cytosol, when compared to delivery of free oligonucleotide or delivery of the oligonucleotide as part of the conjugate comprising the second binding molecule, though in the absence of the conjugate comprising the saponin.

An embodiment is the therapeutic combination of the invention or the pharmaceutical composition of the invention, wherein the effector molecule comprises or consists of at least one proteinaceous molecule, preferably selected from any one or more of a peptide, a protein, an enzyme and a protein toxin.

An embodiment is the therapeutic combination of the invention or the pharmaceutical composition of the invention, wherein the effector molecule comprises or consists of at least one of: urease and Cre-recombinase, a proteinaceous toxin, a ribosome-inactivating protein, a protein toxin, a bacterial toxin, a plant toxin, more preferably selected from any one or more of a viral toxin such as apoptin; a bacterial toxin such as Shiga toxin, Shiga-like toxin, Pseudomonas aeruginosa exotoxin (PE) or exotoxin A of PE, full-length or truncated diphtheria toxin (DT), cholera toxin; a fungal toxin such as alpha-sarcin; a plant toxin including ribosome-inactivating proteins and the A chain of type 2 ribosome-inactivating proteins such as dianthin e.g. dianthin-30 or dianthin-32, saporin e.g. saporin-S3 or saporin-S6, bouganin or de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain; or an animal or human toxin such as frog RNase, or granzyme B or human angiogenin, or any toxic fragment or toxic derivative thereof; preferably the protein toxin is dianthin and/or saporin. As further detailed in the Examples section, the intracellular biological effect of effector molecules such as protein toxins is improved and increased when the conjugate comprising the saponin and the conjugate comprising such an effector molecule are together contacted with the same cell bearing the cell-surface molecule. At a dose of the effector molecule that does not exert the biological effect inside the target cell, when the effector molecule is contacted with the target cell in the absence of the saponin, the efficacy of the effector molecule is improved when the effector molecule and the saponin are contacted with the target cell together. The conjugate comprising the first binding molecule and the saponin comprises for example any one of the binding molecules pertuzumab, trastuzumab, matuzumab, cetuximab, EGF, and/or the conjugate comprising the second binding molecule and the effector molecule comprises for example any one of the binding molecules pertuzumab, trastuzumab, matuzumab, cetuximab, EGF, wherein the first and second binding molecule are different. Typically suitable examples are: the first binding molecule is pertuzumab, the second binding molecule is trastuzumab, or vice versa; the first binding molecule is matuzumab, the second binding molecule is cetuximab, or vice versa; the first binding molecule is matuzumab, the second binding molecule is EGF, or vice versa. Typically, such combinations of a first and second monoclonal antibody are comprised by the conjugates of the invention in combination with an effector molecule selected from an oligonucleotide such as a BNA, or a protein such as an enzyme or a protein toxin. Of course, effector molecules selected from a small-molecule drug molecule are equally suitable, such as effector molecules commonly applied as part of ADCs.

An embodiment is the therapeutic combination of the invention or the pharmaceutical composition of the invention, wherein the effector molecule comprises or consists of at least one payload. Payloads are effector molecules such as small molecules, drug molecules, toxins, small-molecule drugs, peptides, statins, etc. Payloads are typically part of ADCs. Typically, in embodiments, the effector molecule comprises or consists of at least one of: a toxin targeting ribosomes, a toxin targeting elongation factors, a toxin targeting tubulin, a toxin targeting DNA and a toxin targeting RNA, more preferably any one or more of emtansine, pasudotox, maytansinoid derivative DM1, maytansinoid derivative DM4, monomethyl auristatin E (MMAE, vedotin), monomethyl auristatin F (MMAF, mafodotin), a Calicheamicin, N-Acetyl-γ-calicheamicin, a pyrrolobenzodiazepine (PBD) dimer, a benzodiazepine, a CC-1065 analogue, a duocarmycin, Doxorubicin, paclitaxel, docetaxel, cisplatin, cyclophosphamide, etoposide, docetaxel, 5-fluorouracyl (5-FU), mitoxantrone, a tubulysin, an indolinobenzodiazepine, AZ13599185, a cryptophycin, rhizoxin, methotrexate, an anthracycline, a camptothecin analogue, SN-38, DX-8951f, exatecan mesylate, truncated form of Pseudomonas aeruginosa exotoxin (PE38), a Duocarmycin derivative, an amanitin, α-amanitin, a spliceostatin, a thailanstatin, ozogamicin, tesirine, Amberstatin269 and soravtansine, or a derivative thereof.

An embodiment is the therapeutic combination of the invention or the pharmaceutical composition of the invention, wherein the conjugate comprising the second binding molecule and the effector molecule comprises or consists of an antibody-drug conjugate, such as any one of antibody-drug conjugates: gemtuzumab ozogamicin, brentuximab vedotin, trastuzumab emtansine, inotuzumab ozogamicin, moxetumomab pasudotox and polatuzumab vedotin, or comprises or consists of at least the drug and one cell-surface molecule binding-domain of the antibody, and/or comprises or consists of at least the drug and one cell-surface molecule binding-fragment of the antibody. For example, the conjugate is pertuzumab-dianthin, pertuzumab-saporin, trastuzumab-dianthin, trastuzumab-saporin, matuzumab-dianthin, matuzumab-saporin, cetuximab-dianthin, cetuximab-saporin. Of course, it is part of the invention that the antibody is a selected further antibody for example known for its specific binding to e.g. a tumor cell. In addition, it is also part of the invention that the first and second binding molecule can be a domain or fragment of a first and second antibody, such domain or fragment bearing the capability of specifically binding to the different binding sites on the same target cell-surface molecule. Typical fragments and domains are Fab, scFv, single domain antibody such as V_{HH} such as camelid V_{H}, etc.

An embodiment is the therapeutic combination of the invention or the pharmaceutical composition of the invention, wherein the conjugate comprising the first binding molecule and the at least one saponin comprises more than one covalently bound saponin, preferably 2, 3, 4, 5, 6, 8, 10, 16, 32, 64, 128 or 1-100 saponins, or any number of saponins therein between, such as 7, 9, 12 saponins. It is one of the many benefits of the combination and the compositions of the current invention, i.e. the (combination of) conjugates of the invention, that the number of the saponin moieties comprised by the conjugate bearing the first binding molecule, can be adapted to the requirements such as the saponin dose required inside a cell for efficiently supporting and stimulating delivery of the effector molecule inside the cell, and inside the cytosol of said cell. It may be beneficial to couple more than one saponin to the first binding molecule, when for example the cell-surface receptor is expressed to a relatively low extent on the target cell surface. Increasing the number of saponins in the conjugate aids in reaching a sufficiently high intracellular dose of saponin. For example, 2, 4, 8, 16, 32, or 64, or any number therein between, saponins are linked to the first binding molecule. Increasing the number of saponin moieties per conjugate can also result in an efficient dose of the conjugate comprising the saponins which is lower than when for example a single saponin moiety is comprised by the conjugate. A relatively lower dose of a conjugate bearing more than one saponin, compared to the dose required when the conjugate comprises a single saponin moiety, for reaching an intracellular dose effective for delivery of the effector molecule inside the cell and inside the cytosol, may contribute to a lower risk for side effects, when for example the off-target binding of the first binding molecule to the cell-surface molecule on a cell different from the target cell is considered (e.g., when the cell-surface molecule is not a truly unique target cell surface molecule, but is also expressed, e.g. to a lesser extent, on different cells, e.g. non-tumor cells, healthy cells).

An embodiment is the therapeutic combination of the invention or the pharmaceutical composition of the invention, wherein the more than one covalently bound saponins are covalently bound directly to an amino-acid residue of the first binding molecule, preferably to a cysteine and/or to a lysine, and/or are covalently bound via a linker and/or via a cleavable linker. An embodiment is the therapeutic combination of the invention or the pharmaceutical composition of the invention, wherein the more than one covalently bound saponins are part of a covalent saponin conjugate comprising at least one oligomeric molecule or polymeric molecule and the more than one saponin covalently bound thereto, wherein the covalent saponin conjugate is covalently bound to the first binding molecule. Preferably, 1-8 of such covalent saponin conjugates are bound to the first binding molecule, more preferably 2-4 of such of such covalent saponin conjugates. The at least one covalent saponin conjugate is optionally based on a dendron, wherein optionally 1-32 saponins, preferably 2, 3, 4, 5, 6, 8, 10, 16, 32 saponins, or any number of saponins therein between, such as 7, 9, 12 saponins, are covalently bound to the oligomeric molecule or to the polymeric molecule of the at least one covalent saponin conjugate, either directly or via a linker.

Such a covalent saponin conjugate is suitable for coupling more than one saponin to the first binding molecule, for example to a single binding site on the first binding molecule. This way, for example the (partly) blocking of the capability of the first binding molecule to bind to the cell-surface molecule is prevented, which blocking could occur when several saponins are bound to several separate chemical groups on the binding molecule. Furthermore, application of such covalent saponin conjugate provides flexibility and freedom in selecting the number of saponin moieties to be comprised by the conjugate comprising the first binding molecule.

An embodiment is the therapeutic combination of the invention or the pharmaceutical composition of the invention, wherein the at least one saponin is covalently bound to the first binding molecule via a cleavable linker. A typical example of such a cleavable linker is the EMCH linker as detailed here before. In the acidic conditions inside the endosome and lysosome, a saponin coupled to the first binding molecule via such a cleavable linker is released from the conjugate once the conjugate is delivered inside the endosome or lysosome. Free saponin may exert its endosomal escape enhancing activity to an improved extent when delivery of the effector molecule inside the cell, inside the endosome, inside the lysosome, and ultimately for example into the cytosol, is considered.

An embodiment is the therapeutic combination of the invention or the pharmaceutical composition of the invention, wherein the cleavable linker is subject to cleavage under acidic conditions, reductive conditions, enzymatic conditions and/or light-induced conditions, and preferably the cleavable linker comprises a cleavable bond selected from a hydrazone bond and a hydrazide bond subject to cleavage under acidic conditions, and/or a bond susceptible to proteolysis, for example proteolysis by Cathepsin B, and/or a bond susceptible for cleavage under reductive conditions such as a disulfide bond.

An embodiment is the therapeutic combination of the invention or the pharmaceutical composition of the invention, wherein the cleavable linker is subject to cleavage in vivo under acidic conditions as present in endosomes and/or lysosomes of mammalian cells, preferably human cells, preferably at pH 4.0-6.5, and more preferably at pH 5.5. For example, saponin is coupled to the first binding molecule involving a hydrazone bond, such as present when the saponin is linked to the first binding molecule via an EMCH linker, wherein the hydrazone bond is cleaved at the acidic pH, such as in the endosome and lysosome, therewith providing free saponin inside the cell.

An embodiment is the therapeutic combination of the invention or the pharmaceutical composition of the invention, wherein the oligomeric molecule or the polymeric molecule of the covalent saponin conjugate is covalently bound to the first binding molecule, preferably to an amino-acid residue of the binding molecule. In general, it is preferred that both the saponin and the effector molecule in the separate conjugates are bound to the respective first and second binding molecule involving covalent bonds, and are not bound solely based on any one or more of e.g. salt bridges, hydrogen bonds, van der Waals interactions, etc. Conjugates based on covalent bonds are stable and come with a reduced risk for decomposition, e.g. falling apart in the respective binding molecule and saponin or effector molecule, when for example administered to a subject such as a human patient, at a side different from the intracellular space, e.g. the endosome or cytosol of a target cell exposing the target cell-surface molecule. That is to say, covalent conjugates of the invention are in general sufficiently stable in e.g. the blood circulation or tissue, organs, to remain unaltered and intact, such that the target cell bearing the cell-surface molecule can be reached and the saponin and the effector molecule can be delivered intracellularly.

An embodiment is the therapeutic combination of the invention or the pharmaceutical composition of the invention, wherein the at least one saponin is covalently bound to the oligomeric molecule or to the polymeric molecule of the covalent saponin conjugate via a cleavable linker according to the invention, preferably EMCH linker.

An embodiment is the therapeutic combination of the invention or the pharmaceutical composition of the invention, wherein the at least one saponin is covalently bound to the oligomeric molecule or to the polymeric molecule of the covalent saponin conjugate via any one or more of an imine bond, a hydrazone bond, a hydrazide bond, an oxime bond, a 1,3-dioxolane bond, a disulfide bond, a thio-ether bond, an amide bond, a peptide bond or an ester bond, preferably via a linker.

An embodiment is the therapeutic combination of the invention or the pharmaceutical composition of the invention, wherein the at least one saponin comprises an aglycone core structure comprising an aldehyde function in position C₂₃ and the at least one saponin comprising optionally a glucuronic acid function in a first saccharide chain at the C₃beta-OH group of the aglycone core structure of the at least one saponin, which aldehyde function is involved in the covalent bonding to the oligomeric molecule or to polymeric molecule of the covalent saponin conjugate, and/or, if present, the glucuronic acid function is involved in the covalent bonding to the oligomeric molecule or to the polymeric molecule of the covalent saponin conjugate, the bonding of the saponin either via a direct covalent bond, or via a linker.

An embodiment is the therapeutic combination of the invention or the pharmaceutical composition of the invention, wherein the aldehyde function in position C₂₃ of the aglycone core structure of the at least one saponin is covalently bound to linker EMCH, which EMCH is covalently bound via a thio-ether bond to a sulfhydryl group in the oligomeric molecule or in the polymeric molecule of the covalent saponin conjugate, such as a sulfhydryl group of a cysteine. For example, an oligomeric structure or polymeric structure is selected which comprises the number of free sulfhydryl groups matching the number of saponin moieties selected to be comprised by the conjugate comprising the first binding molecule. For example, when the first binding molecule has two binding sites for binding a covalent saponin conjugate, and it is aimed for to provide a conjugate bearing 8 saponin moieties, a polymeric structure comprising four binding sites for coupling of a saponin is selected, such as for example a polymeric molecule bearing four free sulfhydryl groups for linking a saponin via the maleimide group of an EMCH linker, which in turn is coupled to the saponin via a hydrazone bond. Such an oligomeric molecule for example comprises four free cysteine residues.

An embodiment is the therapeutic combination of the invention or the pharmaceutical composition of the invention, wherein the glucuronic acid function in the first saccharide chain at the C₃beta-OH group of the aglycone core structure of the at least one saponin is covalently bound to linker 1-[Bis(dimethylamino)methylene]-1H-1,2,3-triazolo[4,5-b]pyridinium 3-oxid hexafluorophosphate (HATU), which HATU is covalently bound via an amide bond to an amine group in the oligomeric molecule or in the polymeric molecule of the covalent saponin conjugate, such as an amine group of a lysine or an N-terminus of a protein. For example, the oligomeric molecule is a poly-lysine molecule, comprising a selected number of free amine groups for coupling saponins.

An embodiment is the therapeutic combination of the invention or the pharmaceutical composition of the invention, wherein the polymeric molecule or the oligomeric molecule of the covalent saponin conjugate is bound to the first binding molecule, preferably to an amino-acid residue of the first binding molecule, involving a click chemistry group on the polymeric molecule or the oligomeric molecule of the covalent saponin conjugate, the click chemistry group preferably selected from a tetrazine, an azide, an alkene or an alkyne, or a cyclic derivative of these groups, more preferably the click chemistry group is an azide. The benefits of the ease of application of click chemistry when providing the conjugate of the invention will be appreciated by the skilled person. Suitable chemical groups for application in click chemistry type of providing covalent conjugates are known in the art, such as for example in the handbook “Bioconjugate Techniques” (G. T. Hermanson, 3^{rd} Edition, 2013, Elsevier Academic Press).

An embodiment is the therapeutic combination of the invention or the pharmaceutical composition of the invention, wherein the polymeric molecule or the oligomeric molecule of the covalent saponin conjugate comprises a polymeric structure and/or an oligomeric structure selected from: a linear polymer, a branched polymer and/or a cyclic polymer, an oligomer, a dendrimer, a dendron, a dendronized polymer, a dendronized oligomer, a DNA, a polypeptide, a poly-lysine, a poly-ethylene glycol, an oligo-ethylene glycol (OEG), such as OEG₃, OEG₄ and OEG₅, or an assembly of these polymeric structures and/or oligomeric structures which assembly is preferably built up by covalent cross-linking, preferably the polymeric molecule or the oligomeric molecule of the covalent saponin conjugate is a dendron such as a poly-amidoamine (PAMAM) dendrimer. It will be appreciated by the skilled person that such oligomeric molecules and such polymeric molecules are particularly suitable for providing a covalent saponin conjugate bearing a selected number of covalently bound saponins at the oligomeric molecule or polymeric molecule, such as 2, 4, 8, 16, 32, 64 or 128 saponin moieties in the covalent saponin conjugate. In addition, the oligomeric structure or the polymeric structure can be selected based on the aim to couple a single or more of the covalent saponin conjugate(s) to the first binding molecule. That is to say, the chemical group on the covalent saponin conjugate for coupling to the first binding molecule, can be adapted to the availability of one or more chemical groups on the first binding molecule, for binding to such one or more covalent saponin conjugate(s). This way, the invention provides for a combination of conjugates, comprising a conjugate comprising the first binding molecule and a selected number of covalent saponin conjugates, wherein the single or each covalent saponin conjugate comprises a selected number of saponin moieties. Thus, the invention provides for large flexibility with regard to the number of saponin moieties comprised by the conjugate comprising the first binding molecule. Moreover, the type of covalent bond between the saponin or the covalent saponin conjugate and the first binding molecule can be selected from numerous options at wish. Preferred is a cleavable covalent bond, such as a bond cleavable under the acidic conditions as present inside the endosome or lysosome, such that the saponin can be delivered ultimately in free form, not bound to the first binding molecule.

An aspect of the invention relates to the therapeutic combination of the invention or the pharmaceutical composition of the invention, for use as a medicament. The combination of the conjugate comprising the first binding molecule and the saponin and the conjugate comprising the second binding molecule and the effector molecule is for example suitable for use as a medicament, e.g. in the treatment of a cancer in a human subject, when e.g. the effector molecule is an anti-tumor drug molecule, a (protein) toxin, etc., and when an effective amount of the two conjugates is administered to the cancer patient in need of such anti-tumor treatment.

An aspect of the invention relates to the therapeutic combination of the invention or the pharmaceutical composition of the invention, for use in the treatment or prevention of a cancer, an autoimmune disease, a disease relating to (over)expression of a protein, a disease relating to an aberrant cell such as a tumor cell or a diseased liver cell, a disease relating to a mutant gene, a disease relating to a gene defect, a disease relating to a mutant protein, a disease relating to absence of a (functional) protein, a disease relating to a (functional) protein deficiency. As outlined before, the type of selected effector molecule comprised by the conjugate comprising the second binding molecule, is determined by e.g. the availability of known effector molecules which are currently applied in the treatment or prevention of any one or more a cancer, an autoimmune disease, a disease relating to (over)expression of a protein, a disease relating to an aberrant cell such as a tumor cell or a diseased liver cell, a disease relating to a mutant gene, a disease relating to a gene defect, a disease relating to a mutant protein, a disease relating to absence of a (functional) protein, a disease relating to a (functional) protein deficiency. Including such an effector molecule, such as any one of the effector molecules outlined here above, in the conjugate comprising the second binding molecule, and combining said conjugate comprising the selected effector molecule with the conjugate comprising the saponin, provides for the therapeutic combination of the invention or the pharmaceutical composition of the invention that has improved efficacy when compared to treatment of a patient in need thereof with the effector molecule only, either as a free molecule, or as part of e.g. an ADC, AOC. Improved efficacy is here to be understood for example as a desired or sufficient therapeutic effect in the patient to whom the conjugates of the invention are administered, at a lower dose of the effector molecule compared to the dose of the effector molecule when administered in a form other than as part of the conjugate comprising the second binding molecule, which conjugate is administered in combination with the conjugate comprising the saponin.

An embodiment is the therapeutic combination for use of the invention or the pharmaceutical composition for use of the invention, wherein:
said use is in the treatment or prevention of cancer in a human subject; and/or
said use is in the treatment or prophylaxis of cancer in a patient in need thereof, wherein the cell-surface molecule is a tumor-cell surface molecule, preferably a tumor cell-specific surface molecule; and/or
the pharmaceutical combination or the pharmaceutical composition, preferably a therapeutically effective amount of the pharmaceutical combination or the pharmaceutical composition, is administered to a patient in need thereof, preferably a human patient.

One of the several benefits of the pharmaceutical combination or the pharmaceutical composition of the invention is that a desired therapeutic effect in a patient to whom said combination or composition is administered, is for example reached at lower dose of the effector molecule than the dose required when e.g. the effector molecule is administered as part of an ADC, and/or is for example reached to an improved and higher extent than the therapeutic effect that can be reached when the effector molecule is administered to the patient in a different form, e.g. as part of an ADC, AOC, when the therapeutic window of the effector molecule is for example considered. Under influence of the saponin, the effector molecule exerts its intracellular biological effect to a higher extent and/or the desired therapeutic effect of the effector molecule is achieved at a lower dose of the effector molecule administered to the patient in need thereof. Since the first binding molecule and the second binding molecule bind to the same cell-surface molecule, without mutually disturbing the simultaneous binding of the two conjugates comprising the first and second binding molecule, cells relating to a disease, such as tumor cells, that have only a single cell-surface molecule on their surface which is sufficiently specific for the target cell when targeting with therapeutic molecules (i.e. the conjugates of the invention) is considered, can now beneficially targeted by the conjugates of the invention. This provides for treatment options for e.g. cancer patients, not currently available. For example, an ADC comprising the second binding molecule for binding to HER2, CD71 or EGRF, for the treatment of cancer patients with tumors which comprise tumor cells that only expose one of such tumor-cell specific receptors, can now be potentiated by combining the ADC with a conjugate of the invention comprising the first binding site for such tumor-cell receptor and the saponin. Such combination widens the therapeutic window of the ADC. Similarly, an AOC is potentiated.

An aspect of the invention relates to a kit of parts, comprising the pharmaceutical combination of the invention or the pharmaceutical composition of the invention, and optionally instructions for use of said pharmaceutical combination or said pharmaceutical composition. For example, the kit of parts comprises instructions for use of the combination or composition in the treatment or prophylaxis of any of the aforementioned diseases, such as a cancer.

TABLE A1
Saponins displaying (late) endosomal/lysosomal escape enhancing activity, and saponins
comprising a structure reminiscent to such saponins displaying (late) endosomal/
lysosomal escape enhancing activity

| Saponin | | | |
|---|---|---|---|
| Name | Aglycone core | | |
| | | Carbohydrate substituent | Carbohydrate substituent |
| | | at the C-3beta-OH group | at the C-28-OH group |
| NP-005236 | 2alpha- | GlcA- | Glc/Gal- |
| | Hydroxyoleanolic | | |
| | acid | | |
| AMA-1 | 16alpha- | Glc- | Rha-(1→2)-[Xyl-(1→4)]-Rha- |
| | Hydroxyoleanolic | | |
| | acid | | |
| AMR | 16alpha- | Glc- | Rha-(1→2)-[Ara-(1→3)-Xyl-(1→4)]-Rha- |
| | Hydroxyoleanolic | | |
| | acid | | |
| alpha- | Hederagenin (23- | Rha-(1→2)-Ara- | — |
| Hederin | Hydroxyoleanic | | |
| | acid) | | |
| NP-012672 | 16alpha,23-Di- | Ara/Xyl-(1→4)-Rha/Fuc- | Ara/Xyl- |
| | hydroxyoleanolic | (1→2)-Glc/Gal-(1→2)- | |
| | acid | Rha/Fuc-(1→2)-GlcA- | |
| NP-017777 | Gypsogenin | Gal-(1→2)-[Xyl-(1→3)]- | Xyl-(1→4)-Rha-(1→2)-[R-(→4)]-Fuc- |
| | | GlcA- | (R = 4E-Methoxycinnamic acid) |
| NP-017778 | Gypsogenin | Gal-(1→2)-[Xyl-(1→3)]- | Xyl-(1→4)-Rha-(1→2)-[R-(→4)]-Fuc- |
| | | GlcA- | (R = 4Z-Methoxycinnamic acid) |
| NP-017774 | Gypsogenin | Gal-(1→2)-[Xyl-(1→3)]- | Xyl-(1→4)-[Gal-(1→3)]-Rha-(1→2)-4- |
| | | GlcA- | OAc-Fuc- |
| NP-018110^{c}, | Gypsogenin | Gal-(1→2)-[Xyl-(1→3)]- | Xyl-(1→4)-[Glc-(1→3)]-Rha-(1→2)-3,4- |
| NP-017772^{d} | | GlcA- | di-OAc-Fuc- |
| NP-018109 | Gypsogenin | Gal-(1→2)-[Xyl-(1→3)]- | Xyl-(1→4)-(Glc-(1→3)]-Rha-(1→2)-(R-(→4)]-3- |
| | | GlcA- | OAc-Fuc- (R = 4E-Methoxycinnamic acid) |
| NP-017888 | Gypsogenin | Gal-(1→2)-[Xyl-(1→3)]- | Glc-(1→3)-Xyl-(1→4)-[Glc-(1→3)]-Rha-(1→2)-4- |
| | | GlcA- | OAc-Fuc- |
| NP-017889 | Gypsogenin | Gal-(1→2)-[Xyl-(1→3)]- | Glc-(1→3)-Xyl-(1→4)-Rha-(1→2)-4-OAc-Fuc- |
| | | GlcA- | |
| NP-018108 | Gypsogenin | Gal-(1→2)-[Xyl-(1→3)]- | Ara/Xyl-(1→3)-Ara/Xyl-(1→4)-Rha/Fuc- |
| | | GlcA- | (1→2)-[4-OAc-Rha/Fuc-(1→4)]-Rha/Fuc- |
| SA1641^{a}, | Gypsogenin | Gal-(1→2)-[Xyl-(1→3)]- | Xyl-(1→3)-Xyl-(1→4)-Rha-(1→2)-[Qui-(1→4)]- |
| AE X55^{b} | | GlcA- | Fuc- |
| NP-017674 | Quillaic acid | Gal-(1→2)-[Xyl-(1→3)]- | Api-(1→3)-Xyl-(1→4)-[Glc-(1→3)]-Rha-(1→2)- |
| | | GlcA- | Fuc- |
| NP-017810 | Quillaic acid | Gal-(1→2)-[Xyl-(1→3)]- | Xyl-(1→4)-[Gal-(1→3)]-Rha-(1→2)-Fuc- |
| | | GlcA- | |
| AG1 | Quillaic acid | Gal-(1→2)-[Xyl-(1→3)]- | Xyl-(1→4)-[Glc-(1→3)]-Rha-(1→)-Fuc- |
| | | GlcA- | |
| NP-003881 | Quillaic acid | Gal-(1→2)-[Xyl-(1→3)]- | Ara/Xyl-(1→4)-Rha/Fuc-(1→4)-[Glc/Gal-(1→2)]- |
| | | GlcA- | Fuc- |
| NP-017676 | Quillaic acid | Gal-(1→2)-[Xyl-(1→3)]- | Api-(1→3)-Xyl-(1→4)-[Glc-(1→3)]-Rha- |
| | | GlcA- | (1→2)-[R-(→4)]-Fuc- (R = 5-O-[5-O-Ara/Api-3,5- |
| | | | dihydroxy-6-methyl-octanoyl]-3,5-dihydroxy-6- |
| | | | methyl-octanoic acid) |
| NP-017677 | Quillaic acid | Gal-(1→2)-[Xyl-(1→3)]- | Api-(1→3)-Xyl-(1→4)-Rha-(1→2)-[R-(→4)]-Fuc- |
| | | GlcA- | (R = 5-O-[5O-Ara/Api-3,5-dihydroxy-6-methyl- |
| | | | octanoyl]-3,5-dihydroxy-6-methyl-octanoic acid) |
| NP-017706 | Quillaic acid | Gal-(1→2)-[Xyl-(1→3)]- | Api-(1→3)-Xyl-(1→4)-Rha-(1→2)-[Rha-(1→3)]-4- |
| | | GlcA- | OAc-Fuc- |
| NP-017705 | Quillaic acid | Gal-(1→2)-[Xyl-(1→3)]- | Api-(1→3)-Xyl-(1→4)-[Glc-(1→3)]-Rha-(1→2)- |
| | | GlcA- | [Rha-(1→3)]-4-OAc-Fuc- |
| NP-017773 | Quillaic acid | Gal-(1→2)-[Xyl-(1→3)]- | 6-OAc-Glc-(1→3)-Xyl-(1→4)-Rha-(1→2)- |
| | | GlcA- | [3-OAc-Rha-(1→3)]-Fuc- |
| NP-017775 | Quillaic acid | Gal-(1→2)-[Xyl-(1→3)]- | Glc-(1→3)-Xyl-(1→4)-Rha-(1→2)-[3-OAc--Rha- |
| | | GlcA- | (1→3)]-Fuc- |
| SA1657 | Quillaic acid | Gal-(1→2)-[Xyl-(1→3)]- | Xyl-(1→3)-Xyl-(1→4)-Rha-(1→2)-[Qui-(1→4)]-Fuc- |
| | | GlcA- | |
| AG2 | Quillaic acid | Gal-(1→2)-[Xyl-(1→3)]- | Glc-(1→3)-[Xyl-(1→4)]-Rha-(1→2)-[Qui-(1→4)]- |
| | | GlcA- | Fuc- |
| SO1861 | Quillaic acid | Gal-(1→2)-[Xyl-(1→3)]- | Glc-(1→3)-Xyl-(1→4)-Rha-(1→2)-[Xyl-(1→3)-4- |
| | | GlcA- | OAc-Qui-(1→4)]-Fuc- |
| GE1741 | Quillaic acid | Gal-(1→2)-[Xyl-(1→3)]- | Xyl-(1→3)-Xyl-(1→4)-Rha-(1→2)-[3,4-di-OAc-Qui- |
| | | GlcA- | (1→4)]-Fuc- |
| SO1542 | Quillaic acid | Gal-(1→2)-[Xyl-(1→3)]- | Glc-(1→3)-[Xyl-(1→4)]-Rha-(1→2)-Fuc- |
| | | GlcA- | |
| SO1584 | Quillaic acid | Gal-(1→2)-[Xyl-(1→3)]- | 6-OAc-Glc-(1→3)-[Xyl-(1→4)]-Rha-(1→2)-Fuc- |
| | | GlcA- | |
| SO1658 | Gypsogenin | Gal-(1→2)-[Xyl-(1→3)]- | Glc-(1→3)-[Xyl-(1→3)-Xyl-(1→4)]-Rha-(1→2)-Fuc- |
| | | GlcA- | |
| SO1674 | Quillaic acid | Gal-(1→2)-[Xyl-(1→3)]- | Glc-(1→3)-[Xyl-(1→3)-Xyl-(1→4)]-Rha-(1→2)-Fuc- |
| | | GlcA- | |
| SO1832 | Quillaic acid | Gal-(1→2)-[Xyl-(1→3)]- | Xyl-(1→3)-Xyl-(1→4)-Rha-(1→2)-[Xyl-(1→3)-4- |
| | | GlcA- | OAc-Qui-(1→4)]-Fuc- |
| QS-7 (also | Quillaic acid | Gal-(1→2)-[Xyl-(1→3)]- | Api/Xyl-(1→3)-Xyl-(1→4)-[Glc-(1→3)]-Rha-(1→2)- |
| referred to | | GlcA- | [Rha-(1→3)]-4OAc-Fuc- |
| as QS1861) | | | |
| QS-7 api | Quillaic acid | Gal-(1→2)-[Xyl-(1→3)]- | Api-(1→3)-Xyl-(1→4)-[Glc-(1→3)]-Rha-(1→2)- |
| (also | | GlcA- | [Rha-(1→3)]-4OAc-Fuc- |
| referred to | | | |
| as QS1862) | | | |
| QS-17 | Quillaic acid | Gal-(1→2)-[Xyl-(1→3)]- | Api/Xyl-(1→3)-Xyl-(1→4)-[Glc-(1→3)]-Rha-(1→2)- |
| | | GlcA- | [R-(→4)]-Fuc- (R = 5-O-[5-O-Rha-(1→2)-Ara/Api- |
| | | | 3,5-dihydroxy-6-methyl-octanoyl]-3,5-dihydroxy-6- |
| | | | methyl-octanoic acid) |
| QS-18 | Quillaic acid | Gal-(1→2)-[Xyl-(1→3)]- | Api/Xyl-(1→3)-Xyl-(1→4)-[Glc-(1→3)]-Rha-(1→2)- |
| | | GlcA- | [R-(→4)]-Fuc- (R = 5-O-[5-O-Ara/Api-3,5-dihydroxy- |
| | | | 6-melhyl-octanoyl]-3,5-dihydroxy-6-methyl-octanoic |
| | | | acid) |
| QS-21 A- | Quillaic acid | Gal-(1→2)-[Xyl-(1→3)]- | Api-(1→3)-Xyl-(1→4)-Rha-(1→2)-[R-(→4)]-Fuc- |
| apio | | GlcA- | (R = 5-O-[5-O-Ara/Api-3,5-dihydroxy-6-methyl- |
| | | | octanoyl]-3,5-dihydroxy-6-methyl-octanoic acid) |
| QS-21 A- | Quillaic acid | Gal-(1→2)-[Xyl-(1→3)]- | Xyl-(1→3)-Xyl-(1→4)-Rha-(1→2)-[R-(→4)]-Fuc- |
| xylo | | GlcA- | (R = 5-O-[5-O-Ara/Api-3,5-dihydroxy-6-methyl- |
| | | | octanoyl]-3,5-dihydroxy-6-methyl-octanoic acid) |
| QS-21 B- | Quillaic acid | Gal-(1→2)-[Xyl-(1→3)]- | Api-(1→3)-Xyl-(1→4)-Rha-(1→2)-[R-(→3))-Fuc- |
| apio | | GlcA- | (R = 5-O-[5-O-Ara/Api-3,5-dihydroxy-6-methyl- |
| | | | octanoyl]-3,5-dihydroxy-6-methyl-octanoic acid) |
| QS-21 B- | Quillaic acid | Gal-(1→2)-[Xyl-(1→3)]- | Xyl-(1→3)-Xyl-(1→4)-Rha-(1→2)-[R-(→3))-Fuc- |
| xylo | | GlcA- | (R = 5-O-[5-O-Ara/Api-3,5-dihydroxy-6-methyl- |
| | | | octanoyl]-3,5-dihydroxy-6-methyl-octanoic acid) |
| beta-Aescin | Protoaescigenin- | Glc-(1→2)-[Glc-(1→4)]- | — |
| (described: | 21(2-methylbut-2- | GlcA- | |
| Aescin 1a) | enoate)-22-acetat | | |
| Teaseed | 23-Oxo- | Glc-(1→2)-Ara-(1→3)- | — |
| saponin I | barringtogenol | [Gal-(1→2)]-GlcA- | |
| | C-21,22-bis(2- | | |
| | methylbut-2- | | |
| | enoate) | | |
| Teaseed- | 23-Oxo- | Xyl-(1→2)-Ara-(1→3)- | — |
| saponin J | barringtogenol | [Gal-(1→2)]-GlcA- | |
| | C-21,22-bis(2- | | |
| | methylbut-2- | | |
| | enoate) | | |
| Assam- | 23-Oxo- | Glc-(1→2)-Ara-(1→3)- | — |
| saponin F | barringtogenol | [Gal-(1→2)]-GlcA- | |
| | C-21(2-methylbut- | | |
| | 2-enoate)-16,22- | | |
| | diacetat | | |
| Digitonin | Digitogenin | Glc-(1→3)-Gal-(1→2)- | — |
| | | [Xyl-(1→3)]-Glc-(1→4)- | |
| | | Gal- | |
| Primula | 3,16,28-Tri- | Rha-(1→2)-Gal-(1→3)- | — |
| acid 1 | hydroxyoleanan- | [Glc-(1→2)]-GlcA- | |
| | 12-en | | |
| AS64R | Gypsogenic acid | — | Glc-(1→3)-[Glc-(1→6)]-Gal- |
| | | Carbohydrate substituent | |
| | | at the C-23-OH group | |
| AS6.2 | Gypsogenic acid | Gal- | Glc-(1→3)-[Glc-(1→6)]-Gal- |

^{a, b}Different names refer to different isolates of the same structure
^{c, d}Different names refer to different isolates of the same structure

### EXAMPLES AND EXEMPLARY EMBODIMENTS

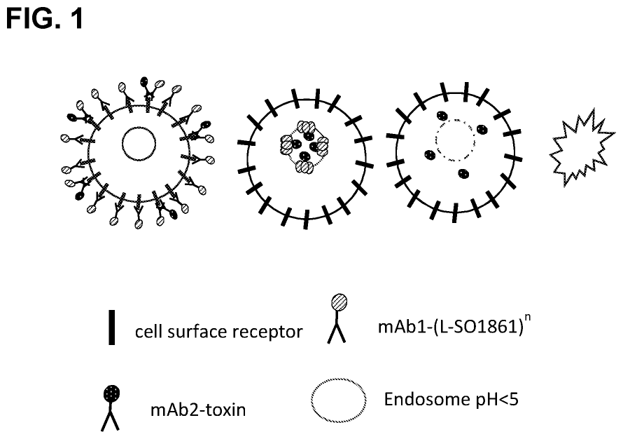

The non-competing 1 target 2-components system (1T2C, non-competing) is the combination treatment of mAb1-SO1861 and mAb2-protein toxin, where mAb1 and mAb2 both target and bind the same receptor, but recognize different epitopes on the receptor, thereby excluding mAb receptor binding competition (FIG. 1). In FIG. 1, a first ligand L1 for binding to the cell-surface molecule (receptor) is bound to a saponin or to more than one saponin moieties, abbreviated as: L1-(S)n, such as the first antibody or first V_{HH} linked to SO1861: mAb1-SO1861. An effector molecule E for exerting a biological effect inside the cell is linked to a second ligand L2 for binding to the same cell-surface molecule (receptor), though to a different binding side than L1: L2-E, such as a protein toxin linked to the second antibody or second V_{HH}: mAb2-toxin. ‘mAb’ refers to monoclonal antibody. In FIG. 1, the ‘mAb1-(L-SO1861)n’ depicts an antibody or V_{HH} bound ton SO1861 moieties via a ‘labile’ linker L, which indicates that the linker L is cleaved in the cell, i.e. at pH as apparent in the endosome and the lysosome.

V_{HH} 7D12 is a single-domain antibody that binds to the (human) receptor for epidermal growth factor (EGFR), which 7D12 has the amino-acid sequence as depicted as SEQ ID NO: 1.

-V_{HH} 7D12
SEQ ID NO: 1
QVKLEESGGGSVQTGGSLRLTCAASGRTSRSYGMGWFRQAPGKEREFVS
GISWRGDSTGYADSVKGRFTISRDNAKNTVDLQMNSLKPEDTAIYYCAA
AAGSAWYGTLYEYDYWGQGTQVTVSS

The single-domain antibody (sdAb) 7D12 [SEQ ID NO: 1] does not compete for binding to the EGFR with the monoclonal antibody matuzumab (R. Heukers et al., Endocytosis of EGFR requires its kinase activity and N-terminal transmembrane dimerization motif (2013), Journal of Cell Science 126, 4900-4912). The combination of sdAb 7D12 and matuzumab, or an EGFR binding domain or EGFR binding fragment of matuzumab, is therefore a typical example of a combination of a first ligand L1 and a non-competing second ligand L2 for binding to the same cell-surface molecule (receptor), though to a different binding side than L1, wherein for example L1 is conjugated with a saponin and L2 is conjugated with an effector moiety, or vice versa. Alternatively, for example an EGFR binding Fab fragment or EGFR binding scFv based on the EGFR binding matuzumab can be applied as a ligand L2 if 7D12 is the first ligand L1. For example, a multivalent ligand L1 is applicable, comprising two or more repeats of the sdAb 7D12, for example two or three linearly conjugated 7D12 domains, wherein L2 is matuzumab or for example an EGFR binding domain or EGFR binding fragment of matuzumab or an EGFR binding Fab fragment or EGFR binding scFv based on the EGFR binding matuzumab.

V_{HH} 9G8 is a single-domain antibody that binds to the (human) receptor for epidermal growth factor (EGFR), which 9G8 has the amino-acid sequence as depicted as SEQ ID NO: 2.

-V_{HH} 9G8
SEQ ID NO: 2
EVQLVESGGGLVQAGGSLRLSCAASGRTFSSYAMGWFRQAPGKEREFVV
AINWSSGSTYYADSVKGRFTISRDNAKNTMYLQMNSLKPEDTAVYYCAA
GYQINSGNYNFKDYEYDYWGQGTQVTVSS

The single-domain antibody (sdAb) 9G8 [SEQ ID NO: 2] does not compete for binding to the EGFR with the monoclonal antibody cetuximab (R. Heukers et al., Endocytosis of EGFR requires its kinase activity and N-terminal transmembrane dimerization motif (2013), Journal of Cell Science 126, 4900-4912). The combination of sdAb 9G8 and cetuximab, or an EGFR binding domain or EGFR binding fragment of cetuximab, is therefore a typical example of a combination of a first ligand L1 and a non-competing second ligand L2 for binding to the same cell-surface molecule (receptor), though to a different binding side than L1, wherein for example L1 is conjugated with a saponin and L2 is conjugated with an effector moiety, or vice versa. Alternatively, for example an EGFR binding Fab fragment or EGFR binding scFv based on the EGFR binding cetuximab can be applied as a ligand L2 if 9G8 is the first ligand L1. For example, a multivalent ligand L1 is applicable, comprising two or more repeats of the sdAb 9G8, for example two or three linearly conjugated 9G8 domains, wherein L2 is cetuximab or for example an EGFR binding domain or EGFR binding fragment of cetuximab or an EGFR binding Fab fragment or EGFR binding scFv based on the EGFR binding cetuximab.

### Example 1. 1T2C, Non-Competing HER2 Targeting

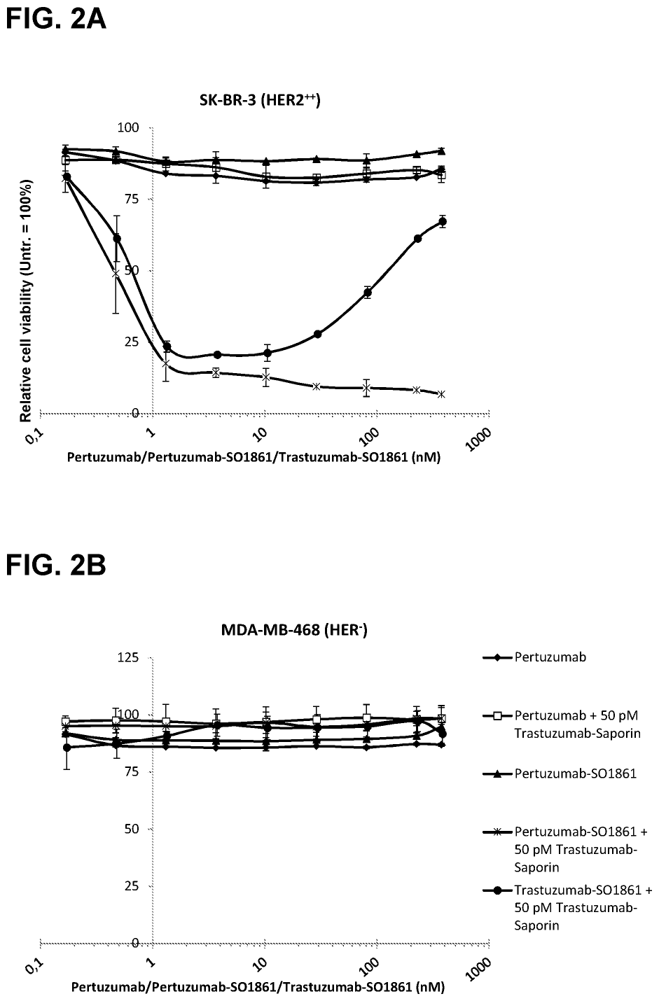

SO1861-EMCH was conjugated via cysteine residues (Cys) to pertuzumab, with a DAR 4, (pertuzumab-(Cys-L-SO1861)⁴. Pertuzumab-(Cys-L-SO1861)⁴ was titrated on a fixed concentration of 50 pM trastuzumab-saporin (trastuzumab conjugated to the protein toxin, saporin, with a DAR4). Pertuzumab and Trastuzumab recognize and bind human HER2 at different epitopes (non-competing). Targeted protein toxin mediated cell killing on HER2 expressing cells (SK-BR-3, HER2⁺⁺) and non-expressing cells (MDA-MB-468, HER2⁻) was determined. This revealed strong cell killing at low and high concentrations of pertuzumab-(Cys-L-SO1861)⁴ (SK-BR-3: IC50=0.5 nM; FIG. 2A) whereas equivalent concentrations pertuzumab, pertuzumab-(Cys-L-SO1861)^{3,9} or pertuzumab+50 pM trastuzumab-saporin could not induce any cell killing activity in HER2 expressing cells. When we compare these data with the combination of Trastuzumab-(Cys-L-SO1861)⁴+50 pM trastuzumab-saporin we observe that at high concentrations Trastuzumab-(Cys-L-SO1861)^{3,9}, cell killing activity is reduced due to competition of both trastuzumab antibody conjugates for binding the HER2 receptor. In MDA-MB-468 cells (no HER2 expression) no cell killing was observed (MDA-MB-468: IC50>1000 nM; FIG. 2B) for any of the treatments.

All this shows that the use of two different antibodies recognizing the same receptor but binding at different epitopes (different binding sites), effectively induce cell killing at low and high concentrations of pertuzumab-(Cys-L-SO1861)⁴ in combination with a fixed low concentration (50 pM) of trastuzumab-saporin in high HER2 expressing cells, but not in cells that do not express HER2. Thus, the use of the combination of both conjugates according to the invention omits competition for receptor binding and reveals activity at low and higher concentrations of pertuzumab-(Cys-L-SO1861)⁴.

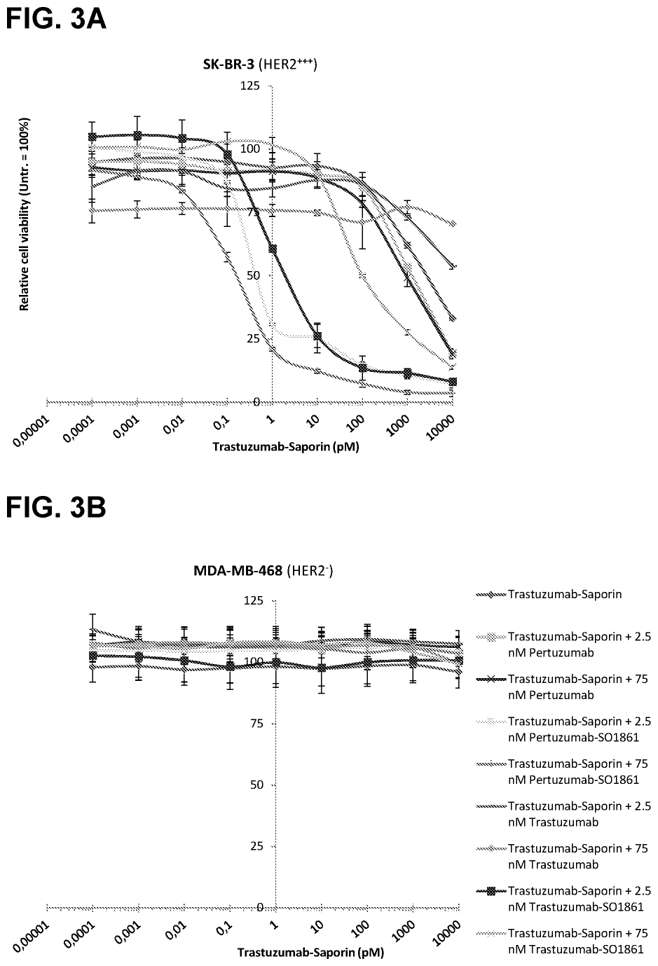

Next, trastuzumab-saporin was titrated on a fixed concentration of 2.5 nM and 75 nM pertuzumab-(Cys-L-SO1861)⁴ and targeted protein toxin mediated cell killing on HER2 expressing cells (SK-BR-3, HER2⁺⁺) and HER2 non-expressing cells (MDA-MB-468, HER2⁻) was determined. This revealed efficient cell killing at low concentrations trastuzumab-saporin in combination with 2.5 nM, or 75 nM pertuzumab-(Cys-L-SO1861)⁴ in SK-BR-3 (HER2⁺⁺; IC50=0.5 pM; FIG. 3A), whereas Trastuzumab-saporin or Trastuzumab-saporin+2.5 nM or 75 nM pertuzumab showed only at high concentrations cell killing in SK-BR-3 cells (IC50>1000 pM; FIG. 3A). When these data is compared with the combination of trastuzumab-saporin+2.5 nM or 75 nM trastuzumab-(Cys-L-SO1861)^{3,9}, cell killing activity was strongly reduced when combined with 75 nM trastuzumab-(Cys-L-SO1861)^{3,9}. In MDA-MB-468 cells (HER2⁻) no cell killing was observed for any of the treatments (MDA-MB-468: IC50>10.000 pM; FIG. 3B).

All this shows that the combination of low concentrations of trastuzumab-saporin+2.5 nM pertuzumab-(Cys-L-SO1861)⁴ or 75 nM pertuzumab-(Cys-L-SO1861)⁴ induce effective cell killing in high HER2 expressing cells. Thus, the use of the combination of both conjugates according to the invention omits competition for receptor binding and reveals effective cell killing at low and higher concentrations of pertuzumab-(Cys-L-SO1861)⁴.

### Example 2. 1T2C, Non-Competing HER2 Targeting

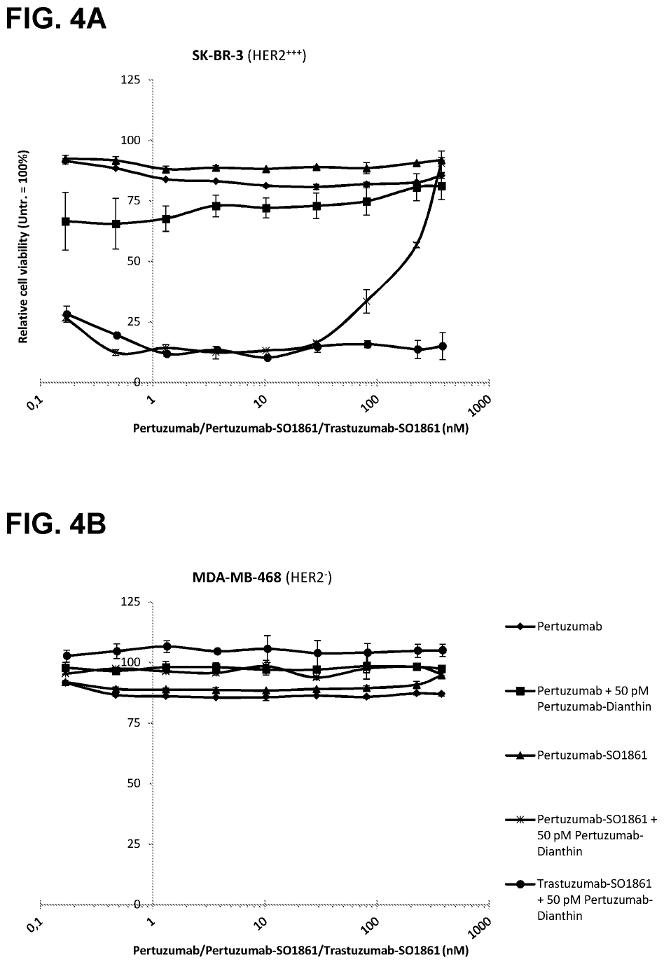

Next, pertuzumab-(Cys-L-SO1861)⁴ or trastuzumab-(Cys-L-SO1861)⁴ was titrated on a fixed concentration of 50 pM pertuzumab-dianthin (pertuzumab conjugated to the protein toxin, dianthin, with a DAR4). Targeted protein toxin mediated cell killing on HER2 expressing cells (SK-BR-3, HER2⁺⁺) and non-expressing cells (MDA-MB-468, HER2⁻) was determined. This revealed strong cell killing at low concentrations of trastuzumab-(Cys-L-SO1861)⁴ or pertuzumab-(Cys-L-SO1861)⁴ (SK-BR-3: IC50<0.1 nM; FIG. 4A). At higher concentrations of pertuzumab-(Cys-L-SO1861)⁴ cell killing activity was reduced whereas higher concentrations of trastuzumab-(Cys-L-SO1861)⁴ were still effective. Equivalent concentrations pertuzumab, pertuzumab-(Cys-L-SO1861)^{3,9} or pertuzumab+50 pM pertuzumab-dianthin were not effective in HER2 expressing cells (SK-BR-3: IC50>1000 nM; FIG. 4A). In MDA-MB-468 cells (HER2⁻) no cell killing was observed (MDA-MB-468: IC50>1000 nM; FIG. 4B) for any of the treatments.

All this shows that the use of two different antibodies recognizing the same receptor but bind at a different epitopes, effectively induce cell killing at low and higher concentrations of trastuzumab-(Cys-L-SO1861)⁴ in combination with a fixed low concentration (50 pM) of pertuzumab-dianthin in high HER2 expressing cells.

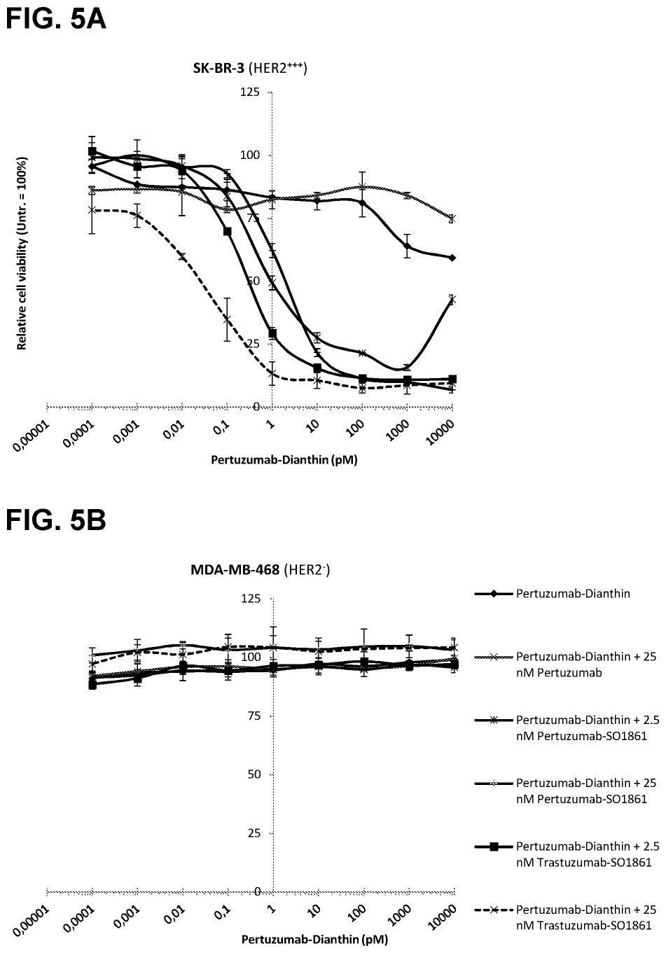

Next, pertuzumab-dianthin was titrated on a fixed concentration of 2.5 nM and 25 nM pertuzumab-(Cys-L-SO1861)⁴ or trastuzumab-(Cys-L-SO1861)⁴ and targeted protein toxin mediated cell killing on HER2 expressing cells (SK-BR-3, HER2⁺⁺) and non-expressing cells (MDA-MB-468, HER2⁻) was determined. This revealed efficient cell killing of SK-BR-3 cells (HER2⁺⁺) at low concentrations pertuzumab-dianthin in combination with 2.5 nM, trastuzumab-(Cys-L-SO1861)⁴ or 2.5 nM pertuzumab-(Cys-L-SO1861)⁴ (IC50=0.5 pM; IC50=0.5 pM, resp. FIG. 5A), whereas pertuzumab-dianthin+25 nM trastuzumab-(Cys-L-SO1861)⁴ showed more efficient cell killing compared to the combination of pertuzumab-dianthin+25 nM pertuzumab-(Cys-L-SO1861)⁴. Equivalent concentrations of pertuzumab-dianthin or pertuzumab-dianthin+25 nM pertuzumab showed only at high concentrations some slight cell killing activity in SK-BR-3 cells (IC50>10.000 pM; FIG. 5A). In MDA-MB-468 cells (HER2⁻) no cell killing was observed for any of the treatments (MDA-MB-468: IC50>10.000 pM; FIG. 5B).

All this shows that the combination according to the invention omits receptor competition, revealing very effective endosomal escape and cytoplasmic toxin delivery resulting in very efficient and selective tumor cell killing.

### Example 3. 1T2C, Non-Competing EGFR Targeting

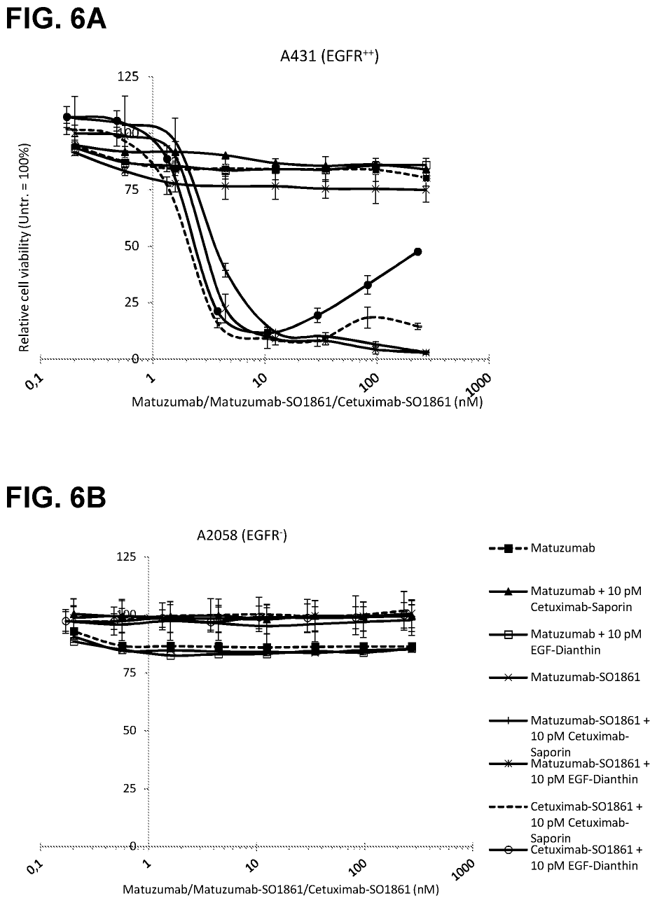

SO1861-EMCH was conjugated via cysteine residues (Cys) to matuzumab, with a DAR 3,3, (matuzumab-SO1861). Matuzumab-SO1861 was titrated on a fixed concentration of 10 pM cetuximab-saporin (cetuximab conjugated to the protein toxin, saporin, with a DAR4) or 10 pM EGFdianthin (recombinant toxin fusion protein). Matuzumab recognizes and binds human EGFR at a different epitope compared to cetuximab and EGF, whereas Cetuximab and EGF compete for binding the EGFR receptor. Targeted protein toxin mediated cell killing on EGFR expressing cells (A431, EGFR⁺⁺) and non-expressing cells (A2058, EGFR⁻) was determined. This revealed strong cell killing at low and higher concentrations of matuzumab-(SO1861)+10 pM cetuximab-saporin or 10 pM EGFdianthin in A431 cells (IC50=2 nM; FIG. 6A) whereas equivalent concentrations matuzumab, matuzumab-SO1861, matuzumab+10 pM cetuximab-saporin or matuzumab+10 pM EGFdianthin could not induce any cell killing activity in A431 cells (IC50>1000 nM; FIG. 6A). When we compare these data with the combination of cetuximab-(Cys-L-SO1861)⁴+10 pM cetuximab-saporin or cetuximab-SO1861+10 pM EGF-dianthin we observe that at higher concentrations Cetuximab-SO1861, cell killing activity is reduced due to competition of both cetuximab conjugate and EGF for binding the EGFR receptor. In A2058 cells (EGFR⁻) no cell killing was observed (IC50>1000 nM; FIG. 6B) for any of the treatments. All this shows that the use of two different antibodies or antibody/ligand combinations recognizing the same receptor but bind at different epitopes, effectively induces cell killing in EGFR⁺⁺ expressing cells at low and high concentrations of matuzumab-SO1861 in combination with a fixed low concentration (10 pM) of cetuximab-saporin or EGFdianthin. Thus the use of the combination according to the invention omits competition for receptor binding and reveals very effective endosomal escape and cytoplasmic toxin delivery resulting in efficient and selective tumor cell killing.

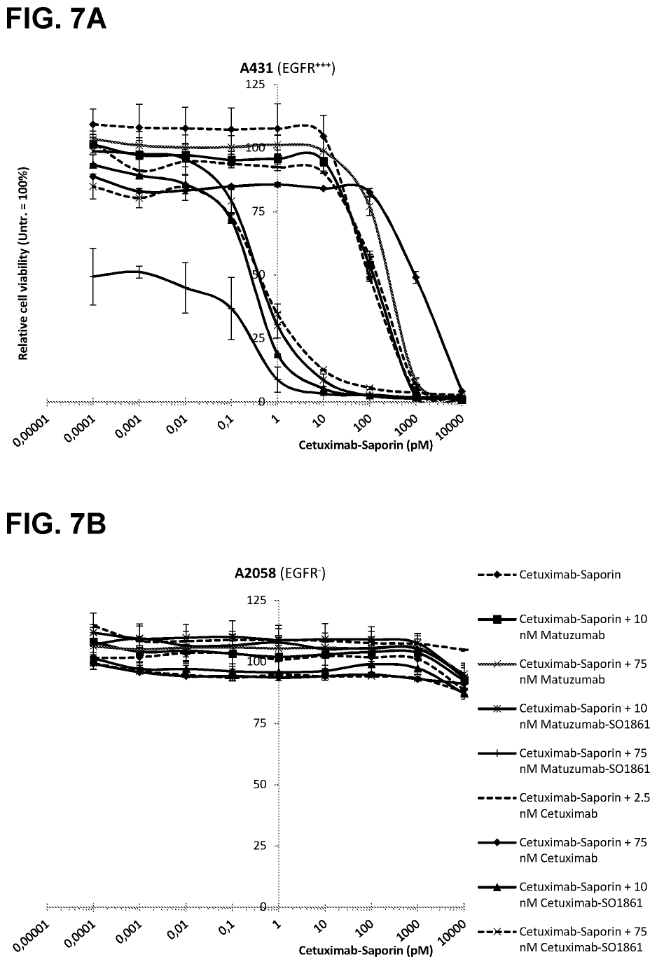

Next, cetuximab-saporin was titrated on a fixed concentration of 10 nM and 75 nM matuzumab-SO1861 and targeted protein toxin mediated cell killing on EGFR expressing cells (A431, EGFR⁺⁺) was determined. This revealed that, 10 nM and 75 nM matuzumab-SO1861 in combination with low concentrations cetuximab-saporin induced efficient cell killing in EGFR expressing cells (A431: 1050=0.5 pM; FIG. 7A), whereas cetuximab-saporin or cetuximab-saporin+10 nM or 75 nM matuzumab showed only at high concentrations cell killing (1050=1000 pM FIG. 3A). When we compared these data with the combination of cetuximab-saporin+10 nM and 75 nM cetuximab-SO1861, cell killing activity was reduced with increased concentrations of cetuximab-SO1861. In A2058 cells (EGFR⁻) no cell killing was observed (A2058: IC50 >1000 pM; FIG. 7B). All this shows that the combination according to the invention omits receptor competition, revealing very effective endosomal escape and cytoplasmic toxin delivery resulting in very efficient and selective tumor cell killing.

### Materials and Methods

SO1861 was isolated and purified by Analyticon Discovery GmbH from raw plant extract obtained from Saponaria officinalis. Matuzumab was sourced from Absolute Antibody Ltd, UK, Trastuzumab (Tras, Herceptin®, Roche), Cetuximab (Cet, Erbitux®, Merck KGaA) and Pertuzumab (purchased from University pharmacy, Berlin) Dianthin-cys was produced and purchased from Proteogenix, France, EGFdianthin was produced from E. coli. according to standard procedures. Cetuximab-saporin and trastuzumab-saporin conjugates were produced and purchased from Advanced Targeting Systems (San Diego, Calif.).

Tris(2-carboxyethyl)phosphine hydrochloride (TCEP, 98%, Sigma-Aldrich), 5,5-Dithiobis(2-nitrobenzoic acid) (DTNB, Ellman's reagent, 99%, Sigma-Aldrich), Zeba™ Spin Desalting Columns (2 mL, Thermo-Fisher), NuPAGE™ 4-12% Bis-Tris Protein Gels (Thermo-Fisher), NuPAGE™ MES SDS Running Buffer (Thermo-Fisher), Novex™ Sharp Pre-stained Protein Standard (Thermo-Fisher), PageBlue™ Protein Staining Solution (Thermo-Fischer), Pierce™ BCA Protein Assay Kit (Thermo-Fisher), N-Ethylmaleimide (NEM, 98%, Sigma-Aldrich), 1,4-Dithiothreitol (DTT, 98%, Sigma-Aldrich), Sephadex G25 (GE Healthcare), Sephadex G50 M (GE Healthcare), Superdex 200P (GE Healthcare), Isopropyl alcohol (IPA, 99.6%, W/R), Tris(hydroxymethyl)aminomethane (Tris, 99%, Sigma-Aldrich), Tris(hydroxymethyl)aminomethane hydrochloride (Tris.HCL, Sigma-Aldrich), L-Histidine (99%, Sigma-Aldrich), D-(+)-Trehalose dehydrate (99%, Sigma-Aldrich), Polyethylene glycol sorbitan monolaurate (TWEEN 20, Sigma-Aldrich), Dulbecco's Phosphate-Buffered Saline (DPBS, Thermo-Fisher), Guanidine hydrochloride (99%, Sigma-Aldrich), Ethylenediaminetetraacetic acid disodium salt dihydrate (EDTA-Naz, 99%, Sigma-Aldrich), sterile filters 0.2 μm and 0.45 μm (Sartorius), Succinimidyl 4-(N-maleimidomethyl)cyclohexane-1-carboxylate (SMCC, Thermo-Fisher), Dianthin-Cys (Dia-Cys, Dianthin mutant with a single C-terminal cysteine was produced by Proteogenix, France), Vivaspin T4 and T15 concentrator (Sartorius), Superdex 200PG (GE Healthcare), Tetra(ethylene glycol) succinimidyl 3-(2-pyridyldithio)propionate (PEG4-SPDP, Thermo-Fisher), HSP27 BNA disulfide oligonucleotide (Biosynthesis), [0-(7-Azabenzotriazol-1-yl)-N,N,N,N-tetramethyluronium-hexafluorphosphat] (HATU, 97%, Sigma-Aldrich), Dimethyl sulfoxide (DMSO, 99%, Sigma-Aldrich), N-(2-Aminoethyl)maleimide trifluoroacetate salt (AEM, 98%, Sigma-Aldrich), L-Cysteine (98.5%, Sigma-Aldrich), deionized water (DI) was freshly taken from Ultrapure Lab Water Systems (MilliQ, Merck), Nickel-nitrilotriacetic acid agarose (Ni-NTA agarose, Protino), Glycine (99.5%, VWR), 5,5-Dithiobis(2-nitrobenzoic acid (Ellman's reagent, DTNB, 98%, Sigma-Aldrich), S-Acetylmercaptosuccinic anhydride Fluorescein (SAMSA reagent, Invitrogen) Sodium bicarbonate (99.7%, Sigma-Aldrich), Sodium carbonate (99.9%, Sigma-Aldrich), PD MiniTrap desalting columns with Sephadex G-25 resin (GE Healthcare), PD10 G25 desalting column (GE Healthcare), Zeba Spin Desalting Columns in 0.5, 2, 5, and 10 mL (Thermo-Fisher), Vivaspin Centrifugal Filters T4 10 kDa MWCO, T4 100 kDa MWCO, and T15 (Sartorius), Biosep s3000 aSEC column (Phenomenex), Vivacell Ultrafiltration Units 10 and 30 kDa MWCO (Sartorius), Nalgene Rapid-Flow filter (Thermo-Fisher),

### SO1861-EMCH Synthesis

To SO1861 (121 mg, 0.065 mmol) and EMCH.TFA (110 mg, 0.325 mmol) was added methanol (extra dry, 3.00 mL) and TFA (0.020 mL, 0.260 mmol). The reaction mixture stirred at room temperature. After 1.5 hours the reaction mixture was subjected to preparative MP-LC.¹ Fractions corresponding to the product were immediately pooled together, frozen and lyophilized overnight to give the title compound (120 mg, 90%) as a white fluffy solid. Purity based on LC-MS 96%.

LRMS (m/z): 2069 [M-1]¹⁻

LC-MS r.t. (min): 1.08⁴

mAb-SO1861 Synthesis
To Matuzumab freshly prepared TCEP solution (1.00 mg/ml, 1.971 mole equivalents, 2.80×10⁻⁵ mmol) was added. The reaction mixture was vortexed briefly then incubated for 90 minutes at 20° C. with roller-mixing. After incubation (prior to addition of SO1861-EMCH), a 0.5 mg (0.101 ml) aliquot of Matuzumab-SH was removed and purified by gel filtration using zeba spin desalting column eluting into TBS pH 7.5. This aliquot was characterised by UV-vis analysis and Ellman's assay. To the bulk Matuzumab-SH was added an aliquot of freshly prepared SO1861-EMCH solution (2.00 mg/ml, 8 mole equivalents, 8.54×10⁻⁵ mmol, 0.089 ml), the mixture vortexed briefly then incubated for 120 minutes at 20° C. Besides the conjugation reaction, two aliquots of desalted Matuzumab-SH (0.10 mg, 0.022 ml, 6.70×10⁻⁷ mmol) were reacted with NEM (8.00 equivalents, 5.36×10⁻⁶ mmol, 0.67 pg, 2.7 μl of a 0.25 mg/ml solution) or TBS pH 7.5 buffer (2.7 μl) for 120 minutes at 20° C., as positive and negative controls, respectively. After incubation (prior to addition of NEM), a ca. 60 pg (0.020 ml) aliquot of Matuzumab-SO1861 mixture was removed and characterised by Ellman's assay alongside positive and negative controls to obtain SO1861 incorporation. To the bulk Matuzumab-SO1861 mixture was added an aliquot of freshly prepared NEM solution (0.25 mg/ml, 5 mole equivalents, 5.34×10⁻⁵ mmol, 0.007 mg) to quench the reaction. The conjugate was purified by zeba 40K MWCO spin column eluting with DPBS pH 7.5 to give purified Matuzumab-SO1861 conjugate. The product was normalised to 2.0 mg/ml and filtered to 0.2 μm, to afford Matuzumab-SO1861 (total yield=1.10 mg, 52%, Matuzumab:SO1861-EMCH=3.3).

Similar procedures were followed to produce pertuzumab-SO1861 (DAR4), cetuximab-SO1861 (DAR4), trastuzumab-SO1861 (DAR4)

### Pertuzumab-Dianthin Synthesis

Dianthin-Cys (17.0 ml, ˜9.6 mg) was concentrated by ultrafiltration using a vivaspin T15 filter tube (3,000 g, 20° C., 10 minutes). The resulting 3.25 ml aliquot was gel filtered using zeba 10 ml spin columns eluting with TBS pH 7.5.

Pertuzumab (0.30 ml, ˜10 mg) was diluted to 10 mg/ml with DPBS pH 7.5, desalted via zeba 5 ml spin column eluting with DPBS pH 7.5 and normalised to 2.50 mg/ml. To an aliquot of Pert (5.00 mg, 3.30×10⁻⁵ mmol, 2.593 mg/ml) was added an aliquot of freshly prepared SMCC solution (1.00 mg/ml, 4.20 mole equivalents, 13.9×10⁻⁵ mmol) in DMSO, the mixture vortexed briefly then incubated for 60 minutes at 20° C. with roller-mixing. After, the reaction was quenched by the addition of an aliquot of a freshly prepared glycine solution (2.0 mg/ml, 5.0 mole equivalents, 69.5×10⁻⁵ mmol) in DPBS pH 7.5. Pert-SMCC (4.27 mg, 2.80×10⁻⁵ mmol, 1.514 mg/ml) was obtained after gel filtration using a zeba 10 ml spin column eluting with TBS pH 7.5.

To Dianthin-Cys (7.54 mg, 25.3×10⁻⁵ mmol, 2.258 mg/ml) was added an aliquot of freshly prepared TCEP solution (1.00 mg/ml, 0.5 mole equivalents, 12.6×10⁻⁵ mmol) in TBS pH 7.5, the mixture briefly vortexed then incubated for 60 minutes at 20° C. with roller-mixing. After, Dianthin-SH (6.0 mg, 20.2×10⁻⁵ mmol, 1.722 mg/ml, Dianthin:SH=1.1) was obtained by gel filtration using a zeba 10 ml spin column eluting with TBS pH 7.5.

To the bulk Pert-SMCC was added the aliquot of Dianthin-SH (7.20 mole equivalents), the mixture vortexed briefly then incubated overnight at 20° C. After ca. 16 hours, the reaction was quenched by the addition of an aliquot of freshly prepared NEM solution (2.50 mg/ml, 5.0 mole equivalents, 101×10⁻⁵ mmol) in TBS pH 7.5. The reaction mixture was filtered to 0.45 μm and then concentrated to <2 ml by ultrafiltration using a vivaspin T15 filter tube (3,000 g, 20° C., 15 minutes). The conjugate was purified by gel filtration using a 1.6×35 cm Superdex 200PG column eluting with DPBS pH 7.5.

### Cell Viability Assay

After treatment the cells were incubated for 72 hr at 37° C. before the cell viability was determined by a MTS-assay, performed according to the manufacturer's instruction (CellTiter 96® AQueous One Solution Cell Proliferation Assay, Promega). Briefly, the MTS solution was diluted 20× in DMEM without phenol red (PAN-Biotech GmbH) supplemented with 10% FBS. The cells were washed once with 200 μL/PBS well, after which 100 μL diluted MTS solution was added/well. The plate was incubated for approximately 20-30 minutes at 37° C. Subsequently, the OD at 492 nm was measured on a Thermo Scientific Multiskan FC plate reader (Thermo Scientific). For quantification the background signal of ‘medium only’ wells was subtracted from all other wells, before the cell viability percentage of treated/untreated cells was calculated, by dividing the background corrected signal of treated wells over the background corrected signal of the untreated wells (×100).

### FACS Analysis

Cells were seeded in DMEM (PAN-Biotech GmbH) supplemented with 10% fetal calf serum (PAN-Biotech GmbH) and 1% penicillin/streptomycin (PAN-Biotech GmbH), at appropriate density for each cell-line in T75 flasks and incubated for 72-84 hrs (5% CO₂, 37° C.), until a confluency of 90% was reached. Next, the cells were trypsinized (TrypIE Express, Gibco Thermo Scientific) to single cells, transferred to a 15 mL falcon tube, and centrifuged (1,400 rpm, 3 min). The supernatant was discarded while leaving the cell pellet submerged. 500.000 Cells were transferred to round bottom FACS tubes and the washed with 3 mL cold PBS (Mg²⁺ and Ca²⁺ free, 2% FBS). The cells were centrifuged at 1800 rpm, 3 min 4° C. and resuspended in 200 μL cold PBS (Mg²⁺ and Ca²⁺ free, 2% FBS) or 200 μL antibody solution; containing 5 μL antibody in 195 μL cold PBS (Mg²⁺ and Ca²⁺ free, 2% FBS). APC Mouse IgG1, κ APC anti-human EGFR (#352906, Biolegend) was used to stain the EGFR receptor. APC anti-human CD340 (erbB2/HER-2) (#324406 Biolegend) was used to stain the HER2 receptor, APC Mouse IgG1a, K Isotype Ctrl FC (#400112, Biolegend) was used for both as its matched isotype control. Samples were incubated for 30 min. at 4° C. on a tube roller mixer. Afterwards, the cells were washed 2× with cold PBS (Mg²⁺ and Ca²⁺ free, 2% FBS) and fixated for 20 min. at room temperature using a 2% PFA solution in PBS (Mg²⁺ and Ca²⁺ free, 2% FBS). Cells were washed 1× with cold PBS, and resuspended in 1000 μL cold PBS for FACS analysis. Samples were analyzed with a BD FACSCanto II flow cytometry system (BD Biosciences) and FlowJo software. The expression levels of EGFR and HER2 of various cells as established with the FACS analyses is summarized in Table A2.

TABLE A2
Expression levels of EGFR,
HER2 of various cells

| | EGFR | HER2 |
|---|---|---|
| | expression | expression |
| Cell line | level (MFI) | level (MFI) |
| MDA-MB-468 | 1656 | 1 |
| A431 | 1593 | 10 |
| SK-BR-3 | 28 | 1162 |
| A2058 | 1 | 5 |

The non-competing 1 target 2-components system (1T2C, non-competing) is the combination treatment of mAb1-SO1861 and mAb2-protein toxin, where mAb1 and mAb2 both target and bind the same receptor, but recognize different epitopes on the receptor, thereby excluding mAb receptor binding competition (FIG. 1). The terms “mAb1” and “mAb2” here refer to a monoclonal antibody and the mAb can also be any binding molecule such as an antibody, an IgG, a binding domain thereof, a binding fragment thereof, a Fab, an scFv, a single-domain antibody (mono-valent, multi-valent such as bi- or tri-valent) such as a V_{HH} domain or a V_{H} domain, etc. Preferred are a monoclonal antibody and a single-domain antibody such as a V_{HH}. For example, mAb1 can be a monoclonal antibody and mAb2 can be a V_{HH}, and vice versa. Any combination of type of mAb1 and type of mAb2 is suitable.

### Example 4. SO1861+EGFR/HER2/CD71 Targeted mAb

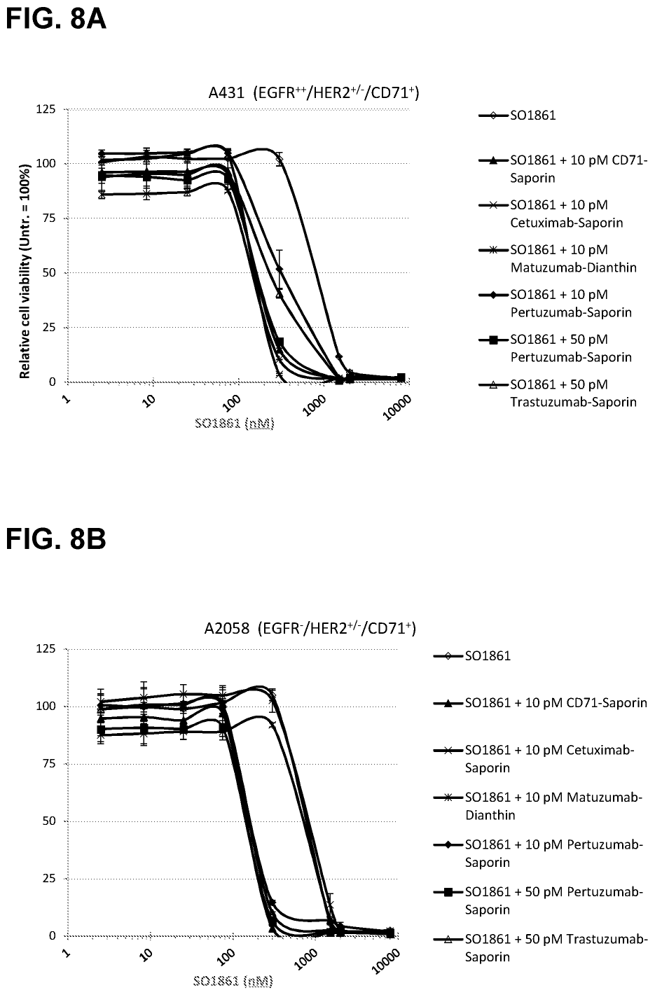

SO1861 was titrated on a fixed concentration of 10 pM CD71-saporin (DAR4), 10 pM cetuximab-saporin (DAR4), 10 pM matuzumab-dianthin (DAR4), 10 pM pertuzumab-saporin (DAR4), 10 pM or 50 pM pertuzumab-saporin (DAR4) and 50 pM trastuzumab-saporin (DAR4) and targeted protein toxin-mediated cell killing on A431 (EGFR⁺⁺/HER2^{+/-}/CD71⁺) and A2058 (EGFR⁻/HER2^{+/-}/CD71⁺) was determined. In A431 cells (EGFR⁺⁺/HER2^{+/-}/CD71⁺) this revealed cell killing activity for all EGFR targeted antibody-toxins (10 pM cetuximab-saporin, and 10 pM matuzumab-dianthin) as well as 10 pM CD71-saporin and 50 pM pertuzumab-saporin at SO1861: IC50=200 nM, whereas 50 pM trastuzumab or 10 pM pertuzumab-saporin showed activity at IC50=250 nM and IC50=300 nM, respectively (FIG. 8A), In A2058 cells (EGFR⁻/HER2^{+/-}/CD71⁺) the EGFR targeted antibody-toxins (10 pM cetuximab-saporin, and 10 pM matuzumab-dianthin) were not active but 10 pM CD71mab-saporin, 10 or 50 pM Pertuzumab-saporin and 50 pM trastuzumab-saporin all showed activity at SO1861: IC50=200 nM (FIG. 8B).

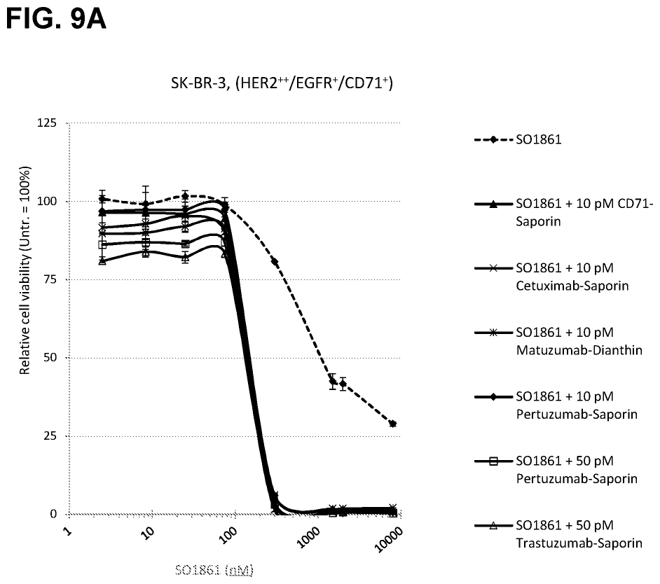

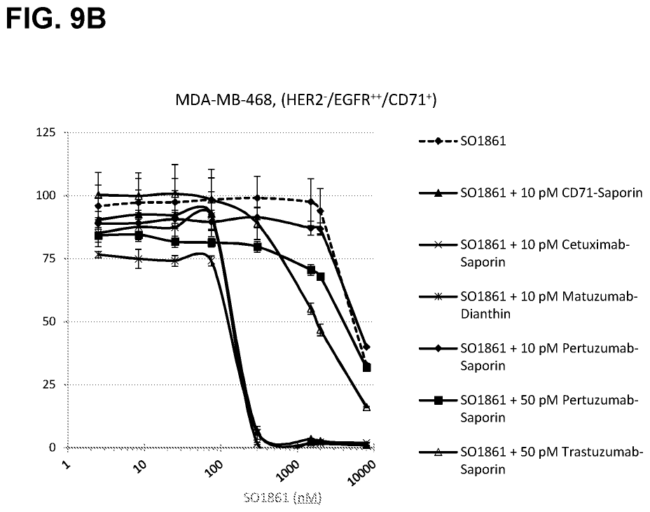

Next, similar experiments were performed on SK-BR-3 cells (HER2⁺⁺/EGFR⁺/CD71⁺) and MDA-MB-468 cells (HER2⁻/EGFR⁺⁺/CD71⁺). In SK-BR-3 cells (HER2⁺⁺/EGFR⁺/CD71⁺) this revealed cell killing activity for all HER2 targeted antibody-toxins (10 or 50 pM Pertuzumab-saporin and 50 pM trastuzumab-saporin) as well as 10 pM CD71-saporin, 10 pM cetuximab-saporin and 10 pM matuzumab-dianthin at SO1861: IC50=200 nM (FIG. 9A). In MDA-MB-468 cells (HER2/EGFR⁺⁺/CD71⁺) the HER2 targeted antibody-toxins (10 or 50 pM Pertuzumab-saporin and 50 pM trastuzumab-saporin) showed only activity at very high concentrations (SO1861: IC50>1000 nM), whereas 10 pM cetuximab-saporin, and 10 pM matuzumab-dianthin and 10 pM CD71mab-saporin showed strong activity at SO1861: IC50=200 nM (FIG. 9B).

### Materials and Methods

SO1861 was isolated and purified by Analyticon Discovery GmbH from raw plant extract obtained from Saponaria officinalis. Matuzumab was sourced from Absolute Antibody Ltd, UK, Trastuzumab (Tras, Herceptin®, Roche), Cetuximab (Cet, Erbitux®, Merck KGaA) and Pertuzumab (purchased from University pharmacy, Berlin), anti-human CD71 (OKT-9), BioXCell. Cetuximab-saporin CD71mab-saporin, pertuzumab-saporin trastuzumab-saporin conjugates were produced and purchased from Advanced Targeting Systems (San Diego, Calif.). Dianthin-cys was produced and purchased from Proteogenix, France, according to standard procedures.

Tris(2-carboxyethyl)phosphine hydrochloride (TCEP, 98%, Sigma-Aldrich), 5,5-Dithiobis(2-nitrobenzoic acid) (DTNB, Ellman's reagent, 99%, Sigma-Aldrich), Zeba™ Spin Desalting Columns (2 mL, Thermo-Fisher), NuPAGE™ 4-12% Bis-Tris Protein Gels (Thermo-Fisher), NuPAGE™ MES SDS Running Buffer (Thermo-Fisher), Novex™ Sharp Pre-stained Protein Standard (Thermo-Fisher), PageBlue™ Protein Staining Solution (Thermo-Fischer), Pierce™ BCA Protein Assay Kit (Thermo-Fisher), N-Ethylmaleimide (NEM, 98%, Sigma-Aldrich), 1,4-Dithiothreitol (DTT, 98%, Sigma-Aldrich), Sephadex G25 (GE Healthcare), Sephadex G50 M (GE Healthcare), Superdex 200P (GE Healthcare), Isopropyl alcohol (IPA, 99.6%, VWR), Tris(hydroxymethyl)aminomethane (Tris, 99%, Sigma-Aldrich), Tris(hydroxymethyl)aminomethane hydrochloride (Tris.HCL, Sigma-Aldrich), L-Histidine (99%, Sig ma-Aldrich), D-(+)-Trehalose dehydrate (99%, Sigma-Aldrich), Polyethylene glycol sorbitan monolaurate (TWEEN 20, Sigma-Aldrich), Dulbecco's Phosphate-Buffered Saline (DPBS, Thermo-Fisher), Guanidine hydrochloride (99%, Sigma-Aldrich), Ethylenediaminetetraacetic acid disodium salt dihydrate (EDTA-Naz, 99%, Sigma-Aldrich), sterile filters 0.2 μm and 0.45 μm (Sartorius), Succinimidyl 4-(N-maleimidomethyl)cyclohexane-1-carboxylate (SMCC, Thermo-Fisher), Dianthin-Cys (Dia-Cys, Dianthin mutant with a single C-terminal cysteine was produced by Proteogenix, France), Vivaspin T4 and T15 concentrator (Sartorius), Superdex 200PG (GE Healthcare), Tetra(ethylene glycol) succinimidyl 3-(2-pyridyldithio)propionate (PEG4-SPDP, Thermo-Fisher), HSP27 BNA disulfide oligonucleotide (Biosynthesis), [0-(7-Azabenzotriazol-1-yl)-N,N,N,N-tetramethyluronium-hexafluorphosphat] (HATU, 97%, Sigma-Aldrich), Dimethyl sulfoxide (DMSO, 99%, Sigma-Aldrich), N-(2-Aminoethyl)maleimide trifluoroacetate salt (AEM, 98%, Sigma-Aldrich), L-Cysteine (98.5%, Sigma-Aldrich), deionized water (DI) was freshly taken from Ultrapure Lab Water Systems (MilliQ, Merck), Nickel-nitrilotriacetic acid agarose (Ni-NTA agarose, Protino), Glycine (99.5%, VWR), 5,5-Dithiobis(2-nitrobenzoic acid (Ellman's reagent, DTNB, 98%, Sigma-Aldrich), S-Acetylmercaptosuccinic anhydride Fluorescein (SAMSA reagent, Invitrogen) Sodium bicarbonate (99.7%, Sigma-Aldrich), Sodium carbonate (99.9%, Sigma-Aldrich), PD MiniTrap desalting columns with Sephadex G-25 resin (GE Healthcare), PD10 G25 desalting column (GE Healthcare), Zeba Spin Desalting Columns in 0.5, 2, 5, and 10 mL (Thermo-Fisher), Vivaspin Centrifugal Filters T4 10 kDa MWCO, T4 100 kDa MWCO, and T15 (Sartorius), Biosep s3000 aSEC column (Phenomenex), Vivacell Ultrafiltration Units 10 and 30 kDa MWCO (Sartorius), Nalgene Rapid-Flow filter (Thermo-Fisher),

### Matuzumab-Dianthin Synthesis

Dianthin-Cys (17.0 ml, ˜9.6 mg) was concentrated by ultrafiltration using a vivaspin T15 filter tube (3,000 g, 20° C., 10 minutes). The resulting 3.25 ml aliquot was gel filtered using zeba 10 ml spin columns eluting with TBS pH 7.5.

Matuzumab (0.30 ml, ˜10 mg) was diluted to 10 mg/ml with DPBS pH 7.5, desalted via zeba 5 ml spin column eluting with DPBS pH 7.5 and normalised to 2.50 mg/ml. To an aliquot of Pert (5.00 mg, 3.30×10⁻⁵ mmol, 2.593 mg/ml) was added an aliquot of freshly prepared SMCC solution (1.00 mg/ml, 4.20 mole equivalents, 13.9×10⁻⁵ mmol) in DMSO, the mixture vortexed briefly then incubated for 60 minutes at 20° C. with roller-mixing. After, the reaction was quenched by the addition of an aliquot of a freshly prepared glycine solution (2.0 mg/ml, 5.0 mole equivalents, 69.5×10⁻⁵ mmol) in DPBS pH 7.5. Pert-SMCC (4.27 mg, 2.80×10⁻⁵ mmol, 1.514 mg/ml) was obtained after gel filtration using a zeba 10 ml spin column eluting with TBS pH 7.5.

To Dianthin-Cys (7.54 mg, 25.3×10⁻⁵ mmol, 2.258 mg/ml) was added an aliquot of freshly prepared TCEP solution (1.00 mg/ml, 0.5 mole equivalents, 12.6×10⁻⁵ mmol) in TBS pH 7.5, the mixture briefly vortexed then incubated for 60 minutes at 20° C. with roller-mixing. After, Dianthin-SH (6.0 mg, 20.2×10⁻⁵ mmol, 1.722 mg/ml, Dianthin:SH=1.1) was obtained by gel filtration using a zeba 10 ml spin column eluting with TBS pH 7.5.

To the bulk Pert-SMCC was added the aliquot of Dianthin-SH (7.20 mole equivalents), the mixture vortexed briefly then incubated overnight at 20° C. After ca. 16 hours, the reaction was quenched by the addition of an aliquot of freshly prepared NEM solution (2.50 mg/ml, 5.0 mole equivalents, 101×10⁻⁵ mmol) in TBS pH 7.5. The reaction mixture was filtered to 0.45 μm and then concentrated to <2 ml by ultrafiltration using a vivaspin T15 filter tube (3,000 g, 20° C., 15 minutes). The conjugate was purified by gel filtration using a 1.6×35 cm Superdex 200PG column eluting with DPBS pH 7.5.

### Cell Viability Assay

After treatment the cells were incubated for 72 hr at 37° C. before the cell viability was determined by a MTS-assay, performed according to the manufacturer's instruction (CellTiter 96® AQueous One Solution Cell Proliferation Assay, Promega). Briefly, the MTS solution was diluted 20× in DMEM without phenol red (PAN-Biotech GmbH) supplemented with 10% FBS. The cells were washed once with 200 μL/PBS well, after which 100 μL diluted MTS solution was added/well. The plate was incubated for approximately 20-30 minutes at 37° C. Subsequently, the OD at 492 nm was measured on a Thermo Scientific Multiskan FC plate reader (Thermo Scientific). For quantification the background signal of ‘medium only’ wells was subtracted from all other wells, before the cell viability percentage of treated/untreated cells was calculated, by dividing the background corrected signal of treated wells over the background corrected signal of the untreated wells (×100).

### FACS Analysis

Cells were seeded in DMEM (PAN-Biotech GmbH) supplemented with 10% fetal calf serum (PAN-Biotech GmbH) and 1% penicillin/streptomycin (PAN-Biotech GmbH), at appropriate density for each cell-line in T75 flasks and incubated for 72-84 hrs (5% CO₂, 37° C.), until a confluency of 90% was reached. Next, the cells were trypsinized (TrypIE Express, Gibco Thermo Scientific) to single cells, transferred to a 15 mL falcon tube, and centrifuged (1,400 rpm, 3 min). The supernatant was discarded while leaving the cell pellet submerged. 500.000 Cells were transferred to round bottom FACS tubes and the washed with 3 mL cold PBS (Mg²⁺ and Ca²⁺ free, 2% FBS). The cells were centrifuged at 1800 rpm, 3 min 4° C. and resuspended in 200 μL cold PBS (Mg²⁺ and Ca²⁺ free, 2% FBS) or 200 μL antibody solution; containing 5 μL antibody in 195 μL cold PBS (Mg²⁺ and Ca²⁺ free, 2% FBS). APC Mouse IgG1, κ APC anti-human EGFR (#352906, Biolegend) was used to stain the EGFR receptor. PE anti-human HER2 APC anti-human CD340 (erbB2/HER-2) (#324408 Biolegend) was used to stain the HER2 receptor, PE Mouse IgG2a, K Isotype Ctrl FC (#400212, Biolegend) was used as its matched isotype control. Samples were incubated for 30 min at 4° C. on a tube roller mixer. Afterwards, the cells were washed 2× with cold PBS (Mg²⁺ and Ca²⁺ free, 2% FBS) and fixated for 20 min at room temperature using a 2% PFA solution in PBS (Mg²⁺ and Ca²⁺ free, 2% FBS). Cells were washed 1× with cold PBS, and resuspended in 1000 μL cold PBS for FACS analysis. Samples were analyzed with a BD FACSCanto II flow cytometry system (BD Biosciences) and FlowJo software. All FACS data see Table A2.

1. Therapeutic combination comprising:
(a) a first pharmaceutical composition comprising a conjugate comprising a first binding molecule comprising a first binding region for binding to a first binding site of a cell-surface molecule and the conjugate comprising at least one saponin covalently bound to said first binding molecule, wherein the saponin is a monodesmosidic triterpene glycoside or a bidesmosidic triterpene glycoside; and
(b) a second pharmaceutical composition comprising a conjugate comprising a second binding molecule different from the first binding molecule, the second binding molecule comprising a second binding region different from the first binding region, the second binding region for binding to a second binding site of said cell-surface molecule different from the first binding site of said cell-surface molecule, and the conjugate comprising an effector molecule covalently bound to said second binding molecule,
the first pharmaceutical composition and the second pharmaceutical composition optionally further comprising a pharmaceutically acceptable excipient and optionally further comprising a pharmaceutically acceptable diluent.

2. Pharmaceutical composition comprising:
a conjugate comprising a first binding molecule comprising a first binding region for binding to a first binding site of a cell-surface molecule and the conjugate comprising at least one saponin covalently bound to said first binding molecule, wherein the saponin is a triterpenoid saponin of the monodesmosidic type or the bidesmosidic type; and
a conjugate comprising a second binding molecule different from said first binding molecule, the second binding molecule comprising a second binding region different from said first binding region, the second binding region for binding to a second binding site of said cell-surface molecule different from said first binding site of said cell-surface molecule, and the conjugate comprising an effector molecule covalently bound to said second binding molecule,
and optionally further comprising a pharmaceutically acceptable excipient and optionally further comprising a pharmaceutically acceptable diluent.

3. The therapeutic combination of claim 1 or the pharmaceutical composition of claim 2, wherein the first binding molecule is a first proteinaceous binding molecule or a first non-proteinaceous ligand comprising the first binding region for binding to the first binding site of the cell-surface molecule, and/or wherein the second binding molecule is a second proteinaceous binding molecule or a second non-proteinaceous ligand comprising the second binding region for binding to the second binding site of the cell-surface molecule.

4. The therapeutic combination of claim 1 or 3 or the pharmaceutical composition of claim 2 or 3, wherein the first binding molecule is a first proteinaceous binding molecule and wherein the saponin is covalently bound to an amino acid residue of the first binding molecule, preferably via a linker.

5. The therapeutic combination of claim 1, 3 or 4 or the pharmaceutical composition of any one of the claims 2-4, wherein the first binding site is a first epitope of said cell-surface molecule such as a cell-surface receptor and wherein the second binding site is a second epitope of said, same, cell-surface molecule, wherein the second epitope is different from the first epitope.

6. The therapeutic combination of any one of the claim 1 or 3-5 or the pharmaceutical composition of any one of the claims 2-5, wherein the saponin is a bidesmosidic triterpene saponin.

7. The therapeutic combination of any one of the claim 1 or 3-6 or the pharmaceutical composition of any one of the claims 2-6, wherein the cell-surface molecule is a tumor-cell surface molecule, preferably a tumor cell-specific cell-surface molecule.

8. The therapeutic combination of any one of the claim 1 or 3-7 or the pharmaceutical composition of any one of the claims 2-7, wherein the first binding region of the first binding molecule comprises or consists of a ligand for binding to the first binding site of the cell-surface molecule such as EGF, or wherein the first binding region of the first binding molecule comprises or consists of an immunoglobulin or at least one binding fragment or binding domain of said immunoglobulin comprising the first binding region for binding to the first binding site of the cell-surface molecule, and/or wherein the second binding region of the second binding molecule comprises or consists of a ligand for binding to the second binding site of the cell-surface molecule such as EGF or a cytokine, or wherein the second binding region of the second binding molecule comprises or consists of an immunoglobulin or at least one binding fragment or binding domain of said immunoglobulin comprising the second binding region for binding to the second binding site of the cell-surface molecule,
wherein the immunoglobulin is preferably any one or more of an antibody such as a monoclonal antibody, preferably a human antibody, an IgG, a molecule comprising or consisting of a single-domain antibody, at least one V_{HH} domain or at least one V_{H} domain, a variable heavy chain new antigen receptor (V_{NAR}) domain, a Fab, an scFv, an Fv, a dAb, an F(ab)2, a Fcab fragment.

9. The therapeutic combination of any one of the claim 1 or 3-8 or the pharmaceutical composition of any one of the claims 2-8, wherein the first binding region of the first binding molecule comprises or consists of a monoclonal antibody, a single-domain antibody, at least one V_{HH} domain, at least one V_{H} domain, a variable heavy chain new antigen receptor (V_{NAR}) domain, a Fab, an scFv, an Fv, a dAb, an F(ab)₂, or a Fcab fragment, preferably a monoclonal antibody or a single-domain antibody, such as at least one V_{HH} domain, and/or wherein the second binding region of the second binding molecule comprises or consists of a monoclonal antibody, a single-domain antibody, at least one V_{HH} domain, at least one V_{H} domain, a variable heavy chain new antigen receptor (V_{NAR}) domain, a Fab, an scFv, an Fv, a dAb, an F(ab)₂, or a Fcab fragment, preferably a monoclonal antibody or a single-domain antibody, such as at least one V_{HH} domain.

10. The therapeutic combination of claim 8 or the pharmaceutical composition of claim 8, wherein the at least one binding fragment or binding domain of said immunoglobulin comprising the first binding region for binding to the first binding site of the cell-surface molecule and/or the at least one binding fragment or binding domain of said immunoglobulin comprising the second binding region for binding to the second binding site of the cell-surface molecule is a single-domain antibody, preferably at least one V_{HH} domain.

11. The therapeutic combination of any one of the claim 1 or 3-10 or the pharmaceutical composition of any one of the claims 2-10, wherein the first binding region and the second binding region are selected to simultaneously bind the same cell-surface molecule at the first binding site and at the second binding site.

12. The therapeutic combination of any one of the claim 1 or 3-11 or the pharmaceutical composition of any one of the claims 2-11, wherein the first binding region is selected to bind to the first binding site of the cell-surface molecule without competing for the binding of the second binding region to the second binding site of the same cell-surface molecule, and wherein the second binding region is selected to bind to the second binding site of the cell-surface molecule without competing for the binding of the first binding region to the first binding site of the same cell-surface molecule.

13. The therapeutic combination of any one of the claim 1 or 3-12 or the pharmaceutical composition of any one of the claims 2-12, wherein the at least one saponin is a bidesmosidic triterpene saponin belonging to the type of a 12,13-dehydrooleanane with an aldehyde function in position C₂₃, the saponin comprising a first saccharide chain at the C₃beta-OH group of the saponin, the first saccharide chain optionally comprising a glucuronic acid moiety, and the saponin comprising a second saccharide chain linked to C₂₈ of the saponin and comprising or consisting of a monosaccharide or a linear or branched oligosaccharide wherein optionally at least one saccharide moiety of the second saccharide chain comprises at least one acetyl group, for example 1, 2, 3 or 4 acetyl groups.

14. The therapeutic combination of any one of the claim 1 or 3-13 or the pharmaceutical composition of any one of the claims 2-13, wherein the at least one saponin is a saponin isolated from any one or more of a Gypsophila species, a Saponaria species, an Agrostemma species and a Quillaja species such as Quillaja saponaria.

15. The therapeutic combination of any one of the claim 1 or 3-14 or the pharmaceutical composition of any one of the claims 2-14, wherein the at least one saponin comprises an aglycone core structure selected from any one or more of:
2alpha-hydroxy oleanolic acid;
16alpha-hydroxy oleanolic acid;
hederagenin (23-hydroxy oleanolic acid);
16alpha,23-dihydroxy oleanolic acid;
gypsogenin;
quillaic acid;
protoaescigenin-21(2-methylbut-2-enoate)-22-acetate;
23-oxo-barringtogenol C-21,22-bis(2-methylbut-2-enoate);
23-oxo-barringtogenol C-21(2-methylbut-2-enoate)-16,22-diacetate;
digitogenin;
3,16,28-trihydroxy oleanan-12-en;
gypsogenic acid; and
a derivative thereof,
preferably, the aglycone core structure is selected from quillaic acid and gypsogenin or a derivative thereof, most preferably the aglycone core structure is quillaic acid or a derivative thereof.

16. Pharmaceutical combination of any one of the claims 1, 3-15 or pharmaceutical composition of any one of the claims 2-15, wherein the at least one saponin comprises a first saccharide chain bound to its aglycone core structure, selected from:
GlcA-,
Glc-,
Gal-,
Rha-(1→2)-Ara-,
Gal-(1→2)-[Xyl-(1→3)]-GlcA-,
Glc-(1→2)-[Glc-(1→4)]-GlcA-,
Glc-(1→2)-Ara-(1→3)-[Gal-(1→2)]-GlcA-,
Xyl-(1→2)-Ara-(1→3)-[Gal-(1→2)]-GlcA-,
Glc-(1→3)-Gal-(1→2)-[Xyl-(1→3)]-Glc-(1→4)-Gal-,
Rha-(1→2)-Gal-(1→3)-[Glc-(1→2)]-GlcA-,
Ara-(1→4)-Rha-(1→2)-Glc-(1→2)-Rha-(1→2)-GlcA-,
Ara-(1→4)-Fuc-(1→2)-Glc-(1→2)-Rha-(1→2)-GlcA-,
Ara-(1→4)-Rha-(1→2)-Gal-(1→2)-Rha-(1→2)-GlcA-,
Ara-(1→4)-Fuc-(1→2)-Gal-(1→2)-Rha-(1→2)-GlcA-,
Ara-(1→4)-Rha-(1→2)-Glc-(1→2)-Fuc-(1→2)-GlcA-,
Ara-(1→4)-Fuc-(1→2)-Glc-(1→2)-Fuc-(1→2)-GlcA-,
Ara-(1→4)-Rha-(1→2)-Gal-(1→2)-Fuc-(1→2)-GlcA-,
Ara-(1→4)-Fuc-(1→2)-Gal-(1→2)-Fuc-(1→2)-GlcA-,
Xyl-(1→4)-Rha-(1→2)-Glc-(1→2)-Rha-(1→2)-GlcA-,
Xyl-(1→4)-Fuc-(1→2)-Glc-(1→2)-Rha-(1→2)-GlcA-,
Xyl-(1→4)-Rha-(1→2)-Gal-(1→2)-Rha-(1→2)-GlcA-,
Xyl-(1→4)-Fuc-(1→2)-Gal-(1→2)-Rha-(1→2)-GlcA-,
Xyl-(1→4)-Rha-(1→2)-Glc-(1→2)-Fuc-(1→2)-GlcA-,
Xyl-(1→4)-Fuc-(1→2)-Glc-(1→2)-Fuc-(1→2)-GlcA-,
Xyl-(1→4)-Rha-(1→2)-Gal-(1→2)-Fuc-(1→2)-GlcA-,
Xyl-(1→4)-Fuc-(1→2)-Gal-(1→2)-Fuc-(1→2)-GlcA-, and
any derivative thereof,
and/or wherein the at least one saponin optionally comprises a second saccharide chain bound to its aglycone core structure, selected from:
Glc-,
Gal-,
Rha-(1→2)-[Xyl-(1→4)]-Rha-,
Rha-(1→2)-[Ara-(1→3)-Xyl-(1→4)]-Rha-,
Ara-,
Xyl-,
Xyl-(1→4)-Rha-(1→2)-[R1-(→4)]-Fuc- wherein R1 is 4E-Methoxycinnamic acid,
Xyl-(1→4)-Rha-(1→2)-[R2-(→4)]-Fuc- wherein R2 is 4Z-Methoxycinnamic acid,
Xyl-(1→4)-[Gal-(1→3)]-Rha-(1→2)-4-OAc-Fuc-,
Xyl-(1→4)-[Glc-(1→3)]-Rha-(1→2)-3,4-di-OAc-Fuc-,
Xyl-(1→4)-[Glc-(1→3)]-Rha-(1→2)-[R3-(→4)]-3-OAc-Fuc- wherein R3 is 4E-Methoxycinnamic acid,
Glc-(1→3)-Xyl-(1→4)-[Glc-(1→3)]-Rha-(1→2)-4-OAc-Fuc-,
Glc-(1→3)-Xyl-(1→4)-Rha-(1→2)-4-OAc-Fuc-,
(Ara- or Xyl-)(1→3)-(Ara- or Xyl-)(1→4)-(Rha- or Fuc-)(1→2)-[4-OAc-(Rha- or Fuc-)(1→4)]-(Rha- or Fuc-),
Xyl-(1→3)-Xyl-(1→4)-Rha-(1→2)-[Qui-(1→4)]-Fuc-,
Api-(1→3)-Xyl-(1→4)-[Glc-(1→3)]-Rha-(1→2)-Fuc-,
Xyl-(1→4)-[Gal-(1→3)]-Rha-(1→2)-Fuc-,
Xyl-(1→4)-[Glc-(1→3)]-Rha-(1→2)-Fuc-,
Ara/Xyl-(1→4)-Rha/Fuc-(1→4)-[Glc/Gal-(1→2)]-Fuc-,
Api-(1→3)-Xyl-(1→4)-[Glc-(1→3)]-Rha-(1→2)-[R4-(→4)]-Fuc- wherein R4 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R5-(→4)]-Fuc- wherein R5 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[Rha-(1→3)]-4-OAc-Fuc-,
Api-(1→3)-Xyl-(1→4)-[Glc-(1→3)]-Rha-(1→2)-[Rha-(1→3)]-4-OAc-Fuc-,
6-OAc-Glc-(1→3)-Xyl-(1→4)-Rha-(1→2)-[3-OAc-Rha-(1→3)]-Fuc-,
Glc-(1→3)-Xyl-(1→4)-Rha-(1→2)-[3-OAc-Rha-(1→3)]-Fuc-,
Xyl-(1→3)-Xyl-(1→4)-Rha-(1→2)-[Qui-(1→4)]-Fuc-,
Glc-(1→3)-[Xyl-(1→4)]-Rha-(1→2)-[Qui-(1→4)]-Fuc-,
Glc-(1→3)-Xyl-(1→4)-Rha-(1→2)-[Xyl-(1→3)-4-OAc-Qui-(1→4)]-Fuc-,
Xyl-(1→3)-Xyl-(1→4)-Rha-(1→2)-[3,4-di-OAc-Qui-(1→4)]-Fuc-,
Glc-(1→3)-[Xyl-(1→4)]-Rha-(1→2)-Fuc-,
6-OAc-Glc-(1→3)-[Xyl-(1→4)]-Rha-(1→2)-Fuc-,
Glc-(1→3)-[Xyl-(1→3)-Xyl-(1→4)]-Rha-(1→2)-Fuc-,
Xyl-(1→3)-Xyl-(1→4)-Rha-(1→2)-[Xyl-(1→3)-4-OAc-Qui-(1→4)]-Fuc-,
Api/Xyl-(1→3)-Xyl-(1→4)-[Glc-(1→3)]-Rha-(1→2)-[Rha-(1→3)]-4OAc-Fuc-,
Api-(1→3)-Xyl-(1→4)-[Glc-(1→3)]-Rha-(1→2)-[Rha-(1→3)]-4OAc-Fuc-,
Api/Xyl-(1→3)-Xyl-(1→4)-[Glc-(1→3)]-Rha-(1→2)-[R6-(→4)]-Fuc- wherein R6 is 5-O-[5-O-Rha-(1→2)-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R7-(→4)]-Fuc- wherein R7 is 5-O-[5-O-Ara/Api-3,5-dihydroxy-6-methyl-octanoyl]-3,5-dihydroxy-6-methyl-octanoic acid),
Api/Xyl-(1→3)-Xyl-(1→4)-[Glc-(1→3)]-Rha-(1→2)-[R8-(→4)]-Fuc- wherein R8 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R9-(→4)]-Fuc- wherein R9 is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R10-(→4)]-Fuc- wherein R10 is 5-O-[5-O-Ara/Api-3,5-dihydroxy-6-methyl-octanoyl]-3,5-dihydroxy-6-methyl-octanoic acid),
Api-(1→3)-Xyl-(1→4)-Rha-(1→2)-[R11-(→3)]-Fuc- wherein R11 is 5-O-[5-O-Ara/Api-3,5-dihydroxy-6-methyl-octanoyl]-3,5-dihydroxy-6-methyl-octanoic acid),
Xyl-(1→3)-Xyl-(1→4)-Rha-(1→2)-[R12-(→3)]-Fuc- wherein R12 is 5-O-[5-O-Ara/Api-3,5-dihydroxy-6-methyl-octanoyl]-3,5-dihydroxy-6-methyl-octanoic acid),
Glc-(1→3)-[Glc-(1→6)]-Gal-, and
a derivative thereof,
preferably the at least one saponin comprises such a first saccharide chain and comprises such a second saccharide chain bound to the aglycone core structure of the saponin of claim 13 or 15.

17. Pharmaceutical combination of any one of the claims 1, 3-16 or pharmaceutical composition of any one of the claims 2-16, wherein the at least one saponin is any one or more of: Quillaja bark saponin, dipsacoside B, saikosaponin A, saikosaponin D, macranthoidin A, esculentoside A, phytolaccagenin, aescinate, AS6.2, NP-005236, AMA-1, AMR, alpha-Hederin, NP-012672, NP-017777, NP-017778, NP-017774, NP-018110, NP-017772, NP-018109, NP-017888, NP-017889, NP-018108, SA1641, AE X55, NP-017674, NP-017810, AG1, NP-003881, NP-017676, NP-017677, NP-017706, NP-017705, NP-017773, NP-017775, SA1657, AG2, SO1861, GE1741, S01542, S01584, S01658, S01674, S01832, SO1862, S01904, QS-7, QS1861, QS-7 api, QS1862, QS-17, QS-18, QS-21 A-apio, QS-21 A-xylo, QS-21 B-apio, QS-21 B-xylo, beta-Aescin, Aescin Ia, Teaseed saponin I, Teaseedsaponin J, Assamsaponin F, Digitonin, Primula acid 1 and AS64R, or a saponin derivative based thereon, or any of their stereoisomers and/or any combinations thereof, preferably any one or more of QS-21, a QS-21 derivative, SO1861, a SO1861 derivative, SA1641, a SA1641 derivative, GE1741 and a GE1741 derivative, more preferably QS-21, a QS-21 derivative, SO1861 or a SO1861 derivative, most preferably SO1861 or a SO1861 derivative.

18. Pharmaceutical combination of any one of the claims 1, 3-17 or pharmaceutical composition of any one of the claims 2-17, wherein the saponin moiety or the saponin derivative moiety in the first conjugate comprises the first saccharide chain and comprises the second saccharide chain, wherein the first saccharide chain comprises more than one saccharide moiety and the second saccharide chain comprises more than one saccharide moiety, and wherein the aglycone core structure of the saponin is, or is a derivative of, quillaic acid or gypsogenin, wherein one, two or three, preferably one or two, of:
i. an aldehyde group in the aglycone core structure of the saponin has been derivatised,
ii. a carboxyl group of a glucuronic acid moiety in the first saccharide chain has been derivatised, and
iii. at least one acetoxy (Me(CO)O—) group in the second saccharide chain has been derivatised.

19. Pharmaceutical combination of any one of the claims 1, 3-18 or pharmaceutical composition of any one of the claims 2-18, wherein the saponin moiety or the saponin derivative moiety in the first conjugate comprises:
i. an aglycone core structure comprising an aldehyde group which has been derivatised by:
reduction to an alcohol;
transformation into a hydrazone bond through reaction with N-ε-maleimidocaproic acid hydrazide (EMCH), wherein the maleimide group of the EMCH is optionally derivatised by formation of a thioether bond with mercaptoethanol;
transformation into a hydrazone bond through reaction with N-[ß-maleimidopropionic acid] hydrazide (BMPH), wherein the maleimide group of the BMPH is optionally derivatised by formation of a thioether bond with mercaptoethanol; or
transformation into a hydrazone bond through reaction with N-[κ-maleimidoundecanoic acid] hydrazide (KMUH), wherein the maleimide group of the KMUH is optionally derivatised by formation of a thioether bond with mercaptoethanol;
ii. a first saccharide chain comprising a carboxyl group, preferably a carboxyl group of a glucuronic acid moiety, which has been derivatised by transformation into an amide bond through reaction with 2-amino-2-methyl-1,3-propanediol (AMPD) or N-(2-aminoethyl)maleimide (AEM);
iii. a second saccharide chain comprising an acetoxy group (Me(CO)O—) which has been derivatised by transformation into a hydroxyl group (HO−) by deacetylation; or
iv. any combination of two or three, preferably two, derivatisations of derivatisations i., ii. and iii.

20. Pharmaceutical combination of any one of the claims 1, 3-19 or pharmaceutical composition of any one of the claims 2-19, wherein the at least one saponin is any one or more of: SO1861, SA1657, GE1741, SA1641, QS-21, QS-21A, QS-21 A-api, QS-21 A-xyl, QS-21B, QS-21 B-api, QS-21 B-xyl, QS-7-xyl, QS-7-api, QS-17-api, QS-17-xyl, QS1861, QS1862, Quillaja saponin, Saponinum album, QS-18, Quil-A, Gyp1, gypsoside A, AG1, AG2, S01542, S01584, S01658, S01674, S01832, or a saponin derivative thereof, or a stereoisomer thereof and/or any combination thereof, preferably any one or more of QS-21 or a QS-21 derivative, SO1861 or a SO1861 derivative, SA1641 or a SA1641 derivative and GE1741 or a GE1741 derivative, more preferably a QS-21 derivative or a SO1861 derivative, most preferably SO1861 or a SO1861 derivative.

21. Pharmaceutical combination of any one of the claims 1, 3-20 or pharmaceutical composition of any one of the claims 2-20, wherein the at least one saponin is a bidesmosidictriterpene glycoside belonging to the type of a 12,13-dehydrooleanane with an aldehyde function in position C₂₃ of the aglycone core structure of the saponin, wherein the saponin is covalently bound to the first binding molecule, preferably covalently bound to an amino-acid residue of the first binding molecule, via an aldehyde function in the saponin, preferably said aldehyde function in position C₂₃ of the aglycone core structure, preferably via at least one linker, and/or via at least one cleavable linker, wherein the amino-acid residue preferably is selected from cysteine and lysine.

22. Pharmaceutical combination of claim 21 or pharmaceutical composition of claim 21, wherein the aldehyde function in position C₂₃ of the aglycone core structure of the at least one saponin is covalently bound to linker EMCH, which linker is covalently bound via a thio-ether bond to a sulfhydryl group in the first binding molecule, such as a sulfhydryl group of a cysteine.

23. Pharmaceutical combination of any one of the claims 1, 3-22 or pharmaceutical composition of any one of the claims 2-22, wherein the at least one saponin is a bidesmosidictriterpene glycoside belonging to the type of a 12,13-dehydrooleanane with an aldehyde function in position C₂₃ of the aglycone core structure of the saponin and comprising a glucuronic acid unit in a first saccharide chain at the C₃beta-OH group of the aglycone core structure of the saponin, wherein the saponin is covalently bound to an amino-acid residue of the first binding molecule via the carboxyl group of the glucuronic acid unit in the first saccharide chain, preferably via a linker, wherein the amino-acid residue preferably is selected from cysteine and lysine.

24. Pharmaceutical combination of claim 23 or pharmaceutical composition of claim 23, wherein the at least one saponin comprises a glucuronic acid unit in its first saccharide chain at the C₃beta-OH group of the aglycone core structure of the at least one saponin, which glucuronic acid unit is covalently bound to linker 1-[Bis(dimethylamino)methylene]-1H-1,2,3-triazolo[4,5-b]pyridinium 3-oxid hexafluorophosphate (HATU), which linker is preferably covalently bound via an amide bond to an amine group in the first binding molecule, such as an amine group of a lysine or an N-terminus of the first binding molecule if the first binding molecule is a first proteinaceous binding molecule.

25. Pharmaceutical combination of any one of the claims 1, 3-24 or pharmaceutical composition of any one of the claims 2-24, wherein the cell-surface molecule is a cell-surface receptor, preferably a tumor-cell specific cell-surface receptor, more preferably a receptor selected from any one or more of: CD71, CA125, EpCAM(17-1A), CD52, CEA, CD44v6, FAP, EGF-IR, integrin, syndecan-1, vascular integrin alpha-V beta-3, HER2, EGFR, CD20, CD22, Folate receptor 1, CD146, CD56, CD19, CD138, CD27L receptor, PSMA, CanAg, integrin-alphaV, CA6, CD33, mesothelin, Cripto, CD3, CD30, CD239, CD70, CD123, CD352, DLL3, CD25, ephrinA4, MUC1, Trop2, CEACAM5, CEACAM6, HER3, CD74, PTK7, Notch3, FGF2, C4.4A, FLT3, CD38, FGFR3, CD7, PD-L1, CTLA4, CD52, PDGFRA, VEGFR1, VEGFR2, most preferably selected from: HER2, CD71 and EGFR.

26. Pharmaceutical combination of any one of the claims 1, 3-25 or pharmaceutical composition of any one of the claims 2-25, wherein the first binding region of the first binding molecule and the second binding region of the second binding molecule comprise or consist of an antibody or a cell-surface molecule binding fragment thereof or cell-surface molecule binding domain(s) thereof and/or comprise or consist of a ligand for binding to the cell-surface molecule, preferably selected from: an anti-CD71 monoclonal antibody such as IgG type OKT-9 and a second anti-CD71antibody; an anti-HER2 monoclonal antibody such as trastuzumab (Herceptin), pertuzumab and a third anti-HER2 monoclonal antibody; an anti-CD20 monoclonal antibody such as rituximab, ofatumumab, tositumomab, obinutuzumab ibritumomab and a fifth anti-CD20 monoclonal antibody; an anti-CA125 monoclonal antibody such as oregovomab and a second anti-CA125 monoclonal antibody; an anti-EpCAM (17-1A) monoclonal antibody such as edrecolomab and a second anti-EpCAM (17-1A) monoclonal antibody; an anti-EGFR monoclonal antibody such as cetuximab, matuzumab, panitumumab, nimotuzumab and a fifth anti-EGFR monoclonal antibody or EGF; an anti-CD30 monoclonal antibody such as brentuximab and a second anti-CD30 antibody; an anti-CD33 monoclonal antibody such as gemtuzumab, huMy9-6 and a third anti-CD33 monoclonal antibody; an anti-vascular integrin alpha-v beta-3 monoclonal antibody such as etaracizumab and a second anti-vascular integrin alpha-v beta-3 antibody; an anti-CD52 monoclonal antibody such as alemtuzumab and a second anti-CD52 antibody; an anti-CD22 monoclonal antibody such as epratuzumab, pinatuzumab, binding fragment (Fv) of anti-CD22 antibody moxetumomab, humanized monoclonal antibody inotuzumab and a fifth anti-CD22 monoclonal antibody; an anti-CEA monoclonal antibody such as labetuzumab and a second anti-CEA monoclonal antibody; an anti-CD44v6 monoclonal antibody such as bivatuzumab and a second anti-CD44v6 monoclonal antibody; an anti-FAP monoclonal antibody such as sibrotuzumab and a second anti-FAB monoclonal antibody; an anti-CD19 monoclonal antibody such as huB4 and a second anti-CD19 monoclonal antibody; an anti-CanAg monoclonal antibody such as huC242 and a second anti-CanAg monoclonal antibody; an anti-CD56 monoclonal antibody such as huN901 and a second anti-CD56 monoclonal antibody; an anti-CD38 monoclonal antibody such as daratumumab, OKT-10 anti-CD38 monoclonal antibody and a third anti-CD38 monoclonal antibody; an anti-CA6 monoclonal antibody such as DS6 and a second anti-CA6 monoclonal antibody; an anti-IGF-1R monoclonal antibody such as cixutumumab, 3B7 and a third anti-CA6 monoclonal antibody; an anti-integrin monoclonal antibody such as CNTO 95 and a second anti-integrin monoclonal antibody; an anti-syndecan-1 monoclonal antibody such as B-B4 and a second anti-syndecan-1 monoclonal antibody; an anti-CD79b monoclonal antibody such as polatuzumab and a second anti-CD79b monoclonal antibody, preferably any one of: trastuzumab and pertuzumab; cetuximab and matuzumab; matuzumab and V_{HH} 7D12 with amino-acid sequence of SEQ ID NO: 1; cetuximab and V_{HH} 9G8 with amino-acid sequence of SEQ ID NO: 2; and EGF and matuzumab,
with the proviso that the first binding region and the second binding region are different and with the proviso that the first binding site and the second binding site are different.

27. Pharmaceutical combination of claim 26 or pharmaceutical composition of claim 26, wherein binding of the first binding region to the first binding site does not compete with binding of the second binding region to the second binding site on the same cell-surface molecule, and vice versa.

28. Pharmaceutical combination of any one of the claims 1, 3-27 or pharmaceutical composition of any one of the claims 2-27, wherein the first binding region of the first binding molecule is capable of binding to the first binding site of the cell-surface receptor and the second binding region of the second binding molecule is capable of binding to the second binding site of the cell-surface receptor, simultaneously.

29. Pharmaceutical combination of any one of the claims 1, 3-28 or pharmaceutical composition of any one of the claims 2-28, wherein the first binding region of the first binding molecule is capable of binding to the first binding site of the cell-surface receptor without blocking the capacity of the second binding region of the second binding molecule to bind to the second binding site of the cell-surface receptor simultaneously, and/or wherein the second binding region of the second binding molecule is capable of binding to the second binding site of the cell-surface receptor without blocking the capacity of the first binding region of the first binding molecule to bind to the first binding site of the cell-surface receptor simultaneously.

30. Pharmaceutical combination of any one of the claims 1, 3-29 or pharmaceutical composition of any one of the claims 2-29, wherein the conjugate comprising the first binding molecule and the conjugate comprising the second molecule can bind to the same cell-surface molecule, simultaneously.

31. Pharmaceutical combination of any one of the claims 1, 3-30 or pharmaceutical composition of any one of the claims 2-30, wherein the effector molecule comprises or consists of at least one of a small molecule such as a drug molecule, a toxin such as a protein toxin, an oligonucleotide such as a BNA, a xeno nucleic acid or an siRNA, an enzyme, a peptide, a protein, or any combination thereof, preferably, the effector molecule is a toxin, an enzyme or an oligonucleotide, more preferably, the effector molecule comprises or consists of at least one of an oligonucleotide, a nucleic acid and a xeno nucleic acid.

32. Pharmaceutical combination of any one of the claims 1, 3-32 or pharmaceutical composition of any one of the claims 2-32, wherein the effector molecule is selected from any one or more of a vector, a gene, a cell suicide inducing transgene, deoxyribonucleic acid (DNA), ribonucleic acid (RNA), anti-sense oligonucleotide (ASO, AON), short interfering RNA (siRNA), anti-microRNA (anti-miRNA), DNA aptamer, RNA aptamer, mRNA, mini-circle DNA, peptide nucleic acid (PNA), phosphoramidate morpholino oligomer (PMO), locked nucleic acid (LNA), bridged nucleic acid (BNA), 2′-deoxy-2′-fluoroarabino nucleic acid (FANA), 2′-O-methoxyethyl-RNA (MOE), 2′-O,4′-aminoethylene bridged nucleic acid, 3′-fluoro hexitol nucleic acid (FHNA), a plasmid, glycol nucleic acid (GNA) and threose nucleic acid (TNA), or a derivative thereof, more preferably a BNA, for example a BNA for silencing HSP27 protein expression or a BNA for silencing apolipoprotein B expression.

33. Pharmaceutical combination of any one of the claims 1, 3-33 or pharmaceutical composition of any one of the claims 2-33, wherein the effector molecule comprises or, when dependent on any one of the claims 1, 3-30, consists of at least one proteinaceous molecule, preferably selected from any one or more of a peptide, a protein, an enzyme and a protein toxin.

34. Pharmaceutical combination of any one of the claims 1, 3-33 or pharmaceutical composition of any one of the claims 2-33, wherein the effector molecule comprises or, when dependent on any one of the claims 1, 3-30, consists of at least one of: urease and Cre-recombinase, a proteinaceous toxin, a ribosome-inactivating protein, a protein toxin, a bacterial toxin, a plant toxin, more preferably selected from any one or more of a viral toxin such as apoptin; a bacterial toxin such as Shiga toxin, Shiga-like toxin, Pseudomonas aeruginosa exotoxin (PE) or exotoxin A of PE, full-length or truncated diphtheria toxin (DT), cholera toxin; a fungal toxin such as alpha-sarcin; a plant toxin including ribosome-inactivating proteins and the A chain of type 2 ribosome-inactivating proteins such as dianthin e.g. dianthin-30 or dianthin-32, saporin e.g. saporin-S3 or saporin-S6, bouganin or de-immunized derivative debouganin of bouganin, shiga-like toxin A, pokeweed antiviral protein, ricin, ricin A chain, modeccin, modeccin A chain, abrin, abrin A chain, volkensin, volkensin A chain, viscumin, viscumin A chain; or an animal or human toxin such as frog RNase, or granzyme B or human angiogenin, or any toxic fragment or toxic derivative thereof; preferably the protein toxin is dianthin and/or saporin.

35. Pharmaceutical combination of any one of the claims 1, 3-34 or pharmaceutical composition of any one of the claims 2-34, wherein the effector molecule comprises or, when dependent on any one of the claims 1, 3-30, consists of at least one payload.

36. Pharmaceutical combination of any one of the claims 1, 3-35 or pharmaceutical composition of any one of the claims 2-35, wherein the effector molecule comprises or, when dependent on any one of the claims 1, 3-30, consists of at least one of: a toxin targeting ribosomes, a toxin targeting elongation factors, a toxin targeting tubulin, a toxin targeting DNA and a toxin targeting RNA, more preferably any one or more of emtansine, pasudotox, maytansinoid derivative DM1, maytansinoid derivative DM4, monomethyl auristatin E (MMAE, vedotin), monomethyl auristatin F (MMAF, mafodotin), a Calicheamicin, N-Acetyl-γ-calicheamicin, a pyrrolobenzodiazepine (PBD) dimer, a benzodiazepine, a CC-1065 analogue, a duocarmycin, Doxorubicin, paclitaxel, docetaxel, cisplatin, cyclophosphamide, etoposide, docetaxel, 5-fluorouracyl (5-FU), mitoxantrone, a tubulysin, an indolinobenzodiazepine, AZ13599185, a cryptophycin, rhizoxin, methotrexate, an anthracycline, a camptothecin analogue, SN-38, DX-8951f, exatecan mesylate, truncated form of Pseudomonas aeruginosa exotoxin (PE38), a Duocarmycin derivative, an amanitin, α-amanitin, a spliceostatin, a thailanstatin, ozogamicin, tesirine, Amberstatin269 and soravtansine, or a derivative thereof.

37. Pharmaceutical combination of any one of the claims 1, 3-36 or pharmaceutical composition of any one of the claims 2-36, wherein the conjugate comprising the second binding molecule and the effector molecule comprises or, when dependent on any one of the claims 1, 3-30, consists of an antibody-drug conjugate, such as any one of antibody-drug conjugates: gemtuzumab ozogamicin, brentuximab vedotin, trastuzumab emtansine, inotuzumab ozogamicin, moxetumomab pasudotox and polatuzumab vedotin, or comprises or consists of at least the drug and one cell-surface molecule binding-domain of the antibody, and/or comprises or consists of at least the drug and one cell-surface molecule binding-fragment of the antibody.

38. Pharmaceutical combination of any one of the claims 1, 3-37 or pharmaceutical composition of any one of the claims 2-37, wherein the conjugate comprising the first binding molecule and the at least one saponin comprises more than one covalently bound saponin, preferably 2, 3, 4, 5, 6, 8, 10, 16, 32, 64, 128 or 1-100 saponins, or any number of saponins therein between, such as 7, 9, 12 saponins.

39. Pharmaceutical combination of claim 38 or pharmaceutical composition of claim 38, wherein the more than one covalently bound saponins are covalently bound directly to an amino-acid residue of the first binding molecule, preferably to a cysteine and/or to a lysine, and/or are covalently bound via a linker and/or via a cleavable linker and/or are part of a covalent saponin conjugate comprising at least one oligomeric molecule or polymeric molecule and the more than one saponin covalently bound thereto, wherein the covalent saponin conjugate is covalently bound to the first binding molecule, preferably 1-8 of such covalent saponin conjugates are bound to the first binding molecule, more preferably 2-4 of such of such covalent saponin conjugates, wherein the at least one covalent saponin conjugate is optionally based on a dendron, wherein optionally 1-32 saponins, preferably 2, 3, 4, 5, 6, 8, 10, 16, 32 saponins, or any number of saponins therein between, such as 7, 9, 12 saponins, are covalently bound to the oligomeric molecule or to the polymeric molecule of the at least one covalent saponin conjugate, either directly or via a linker.

40. Pharmaceutical combination of any one of the claims 1, 3-39 or pharmaceutical composition of any one of the claims 2-39, wherein the at least one saponin is covalently bound to the first binding molecule via a cleavable linker.

41. Pharmaceutical combination of claim 40 or pharmaceutical composition of claim 40, wherein the cleavable linker is subject to cleavage under acidic conditions, reductive conditions, enzymatic conditions and/or light-induced conditions, and preferably the cleavable linker comprises a cleavable bond selected from a hydrazone bond and a hydrazide bond subject to cleavage under acidic conditions, and/or a bond susceptible to proteolysis, for example proteolysis by Cathepsin B, and/or a bond susceptible for cleavage under reductive conditions such as a disulfide bond.

42. Pharmaceutical combination of claim 40 or 41 or pharmaceutical composition of claim 40 or 41, wherein the cleavable linker is subject to cleavage in vivo under acidic conditions as present in endosomes and/or lysosomes of mammalian cells, preferably human cells, preferably at pH 4.0-6.5, and more preferably at pH≤5.5.

43. Pharmaceutical combination of claim 39 or any one of claims 40-42 when dependent on claim 39, or pharmaceutical composition of claim 39 or any one of claims 40-42 when dependent on claim 39, wherein the oligomeric molecule or the polymeric molecule of the covalent saponin conjugate is covalently bound to the first binding molecule, preferably to an amino-acid residue of the binding molecule.

44. Pharmaceutical combination of claim 42 or 43, or pharmaceutical composition of claim 42 or 43, wherein the at least one saponin is covalently bound to the oligomeric molecule or to the polymeric molecule of the covalent saponin conjugate via a cleavable linker according to any one of the claims 39-42.

45. Pharmaceutical combination of any one of the claims 42-44, or pharmaceutical composition of any one of the claims 42-44, wherein the at least one saponin is covalently bound to the oligomeric molecule or to the polymeric molecule of the covalent saponin conjugate via any one or more of an imine bond, a hydrazone bond, a hydrazide bond, an oxime bond, a 1,3-dioxolane bond, a disulfide bond, a thio-ether bond, an amide bond, a peptide bond or an ester bond, preferably via a linker.

46. Pharmaceutical combination of any one of the claims 39-45, or pharmaceutical composition of any one of the claims 39-45, wherein the at least one saponin comprises an aglycone core structure comprising an aldehyde function in position C₂₃ and the at least one saponin comprising optionally a glucuronic acid function in a first saccharide chain at the C₃beta-OH group of the aglycone core structure of the at least one saponin, which aldehyde function is involved in the covalent bonding to the oligomeric molecule or to polymeric molecule of the covalent saponin conjugate, and/or, if present, the glucuronic acid function is involved in the covalent bonding to the oligomeric molecule or to the polymeric molecule of the covalent saponin conjugate, the bonding of the saponin either via a direct covalent bond, or via a linker.

47. Pharmaceutical combination of claim 46 or pharmaceutical composition of claim 46, wherein the aldehyde function in position C₂₃ of the aglycone core structure of the at least one saponin is covalently bound to linker EMCH, which EMCH is covalently bound via a thio-ether bond to a sulfhydryl group in the oligomeric molecule or in the polymeric molecule of the covalent saponin conjugate, such as a sulfhydryl group of a cysteine.

48. Pharmaceutical combination of claim 46 or 47 or pharmaceutical composition of claim 46 or 47, wherein the glucuronic acid function in the first saccharide chain at the C₃beta-OH group of the aglycone core structure of the at least one saponin is covalently bound to linker 1-[Bis(dimethylamino)methylene]-1H-1,2,3-triazolo[4,5-b]pyridinium 3-oxid hexafluorophosphate (HATU), which HATU is covalently bound via an amide bond to an amine group in the oligomeric molecule or in the polymeric molecule of the covalent saponin conjugate, such as an amine group of a lysine or an N-terminus of a protein.

49. Pharmaceutical combination of any one of the claims 43-48, or pharmaceutical composition of any one of the claims 43-48, wherein the polymeric molecule or the oligomeric molecule of the covalent saponin conjugate is bound to the first binding molecule, preferably to an amino-acid residue of the first binding molecule, involving a click chemistry group on the polymeric molecule or the oligomeric molecule of the covalent saponin conjugate, the click chemistry group preferably selected from a tetrazine, an azide, an alkene or an alkyne, or a cyclic derivative of these groups, more preferably the click chemistry group is an azide.

50. Pharmaceutical combination of any one of the claims 43-49, or pharmaceutical composition of any one of the claims 43-49, wherein the polymeric molecule or the oligomeric molecule of the covalent saponin conjugate comprises a polymeric structure and/or an oligomeric structure selected from: a linear polymer, a branched polymer and/or a cyclic polymer, an oligomer, a dendrimer, a dendron, a dendronized polymer, a dendronized oligomer, a DNA, a polypeptide, a poly-lysine, a poly-ethylene glycol, an oligo-ethylene glycol (OEG), such as OEG₃, OEG₄ and OEG₅, or an assembly of these polymeric structures and/or oligomeric structures which assembly is preferably built up by covalent cross-linking, preferably the polymeric molecule or the oligomeric molecule of the covalent saponin conjugate is a dendron such as a poly-amidoamine (PAMAM) dendrimer.

51. Pharmaceutical combination of any one of the claims 1-50, or pharmaceutical composition of any one of the claims 1-50, for use as a medicament.

52. Pharmaceutical combination of any one of the claims 1-50, or pharmaceutical composition of any one of the claims 1-50, for use in the treatment or prevention of a cancer, an autoimmune disease, a disease relating to (over)expression of a protein, a disease relating to an aberrant cell such as a tumor cell or a diseased liver cell, a disease relating to a mutant gene, a disease relating to a gene defect, a disease relating to a mutant protein, a disease relating to absence of a (functional) protein, a disease relating to a (functional) protein deficiency.

53. Pharmaceutical combination for use of claim 52, or pharmaceutical composition for use of claim 52, wherein:
said use is in the treatment or prevention of cancer in a human subject; and/or
said use is in the treatment or prophylaxis of cancer in a patient in need thereof, wherein the cell-surface molecule is a tumor-cell surface molecule, preferably a tumor cell-specific surface molecule; and/or
the pharmaceutical combination or the pharmaceutical composition, preferably a therapeutically effective amount of the pharmaceutical combination or the pharmaceutical composition, is administered to a patient in need thereof, preferably a human patient.

54. Kit of parts, comprising the pharmaceutical combination of any one of the claims 1, 3-50 or the pharmaceutical composition of any one of the claims 2-50, and optionally instructions for use of said pharmaceutical combination or said pharmaceutical composition.
